SEQUENCE LISTING (23 sequences)

SEQ ID NO: 1 - protein, 20 aa, synthetic construct
  REGION 1..20: note = Description of Artificial Sequence: Syntheticpeptide
  MOD_RES 10: note = D-amino acid
VKVKVKVKVPPTKVKVKVKV

SEQ ID NO: 2 - protein, 20 aa, synthetic construct
  REGION 1..20: note = Description of Artificial Sequence: Syntheticpeptide
  MOD_RES 10: note = D-amino acid
VKVRVRVRVPPTRVRVRVKV

SEQ ID NO: 3 - protein, 26 aa, synthetic construct
  REGION 1..26: note = Description of Artificial Sequence: Syntheticpeptide
  MOD_RES 10: note = D-amino acid
VKVRVRVRVPPTRVRVRVKVGRGDSP

SEQ ID NO: 4 - protein, 20 aa, synthetic construct
  REGION 1..20: note = Description of Artificial Sequence: Syntheticpeptide
  MOD_RES 10: note = D-amino acid
VRVRVKVRVPPTRVEVRVRV

SEQ ID NO: 5 - protein, 20 aa, synthetic construct
  REGION 1..20: note = Description of Artificial Sequence: Syntheticpeptide
  MOD_RES 10: note = D-amino acid
VRVRVRVKVPPTKVRVRVRV

SEQ ID NO: 6 - protein, 24 aa, synthetic construct
  REGION 1..24: note = Description of Artificial Sequence: Syntheticpeptide
  MOD_RES 14: note = D-amino acid
RGDSVRVRVRVKVPPTKVRVRVRV

SEQ ID NO: 7 - protein, 36 aa, synthetic construct
  REGION 1..36: note = Description of Artificial Sequence: Syntheticpolypeptide
  MOD_RES 10: note = D-amino acid
VKVRVRVRVPPTRVRVRVKVGKLTWQELYQLKYKGI

SEQ ID NO: 8 - protein, 31 aa, synthetic construct
  REGION 1..31: note = Description of Artificial Sequence: Syntheticpolypeptide
  MOD_RES 10: note = D-amino acid
VKVRVRVRVPPTRVRVRVKVRPKPQQFFGLM

SEQ ID NO: 9 - protein, 17 aa, synthetic construct
  REGION 1..17: note = Description of Artificial Sequence: Syntheticpeptide
VKVRVRVRVRVRVRVKV

SEQ ID NO: 10 - protein, 20 aa, synthetic construct
  REGION 1..20: note = Description of Artificial Sequence: Syntheticpeptide
  MOD_RES 10: note = D-amino acid
VKVKVRVKVPPTKVKVRVKV

SEQ ID NO: 11 - protein, 20 aa, synthetic construct
  REGION 1..20: note = Description of Artificial Sequence: Syntheticpeptide
VKVRVRVRYNGTRVRVRVKV

SEQ ID NO: 12 - protein, 28 aa, synthetic construct
  REGION 1..28: note = Description of Artificial Sequence: Syntheticpeptide
  MOD_RES 18: note = D-amino acid
LKKTETQGVKVRVRVRVPPTRVRVRVKV

SEQ ID NO: 13 - protein, 25 aa, synthetic construct
  REGION 1..25: note = Description of Artificial Sequence: Syntheticpeptide
  MOD_RES 15: note = D-amino acid
SDKPGVLVRVRVRVPPTRVRVRVKV

SEQ ID NO: 14 - protein, 24 aa, synthetic construct
  REGION 1..24: note = Description of Artificial Sequence: Syntheticpeptide
  MOD_RES 14: note = D-amino acid
RGDSVRVRVRVKVPPTKVRVRVRV

SEQ ID NO: 15 - protein, 20 aa, synthetic construct
  REGION 1..20: note = Description of Artificial Sequence: Syntheticpeptide
  MOD_RES 10: note = D-amino acid
VKVKVKVKVPPTKVEVKVKV

SEQ ID NO: 16 - protein, 20 aa, synthetic construct
  REGION 1..20: note = Description of Artificial Sequence: Syntheticpeptide
  MOD_RES 10: note = D-amino acid
VLTKVKTKVPPTKVEVKVLV

SEQ ID NO: 17 - protein, 28 aa, synthetic construct
  REGION 1..28: note = Description of Artificial Sequence: Syntheticpeptide
  MOD_RES 10: note = D-amino acid
VLTKVKTKVPPTKVEVKVLVGGGGRGDV

SEQ ID NO: 18 - protein, 24 aa, synthetic construct
  REGION 1..24: note = Description of Artificial Sequence: Syntheticpeptide
  MOD_RES 14: note = D-amino acid
RGDSVKVRVRVRVPPTRVRVRVKV

SEQ ID NO: 19 - protein, 26 aa, synthetic construct
  REGION 1..26: note = Description of Artificial Sequence: Syntheticpeptide
  MOD_RES 16: note = D-amino acid
IKVAVGVKVRVRVRVPPTRVRVRVKV

SEQ ID NO: 20 - protein, 26 aa, synthetic construct
  REGION 1..26: note = Description of Artificial Sequence: Syntheticpeptide
  MOD_RES 16: note = D-amino acid
YIGSRGVKVRVRVRVPPTRVRVRVKN

SEQ ID NO: 21 - protein, 18 aa, synthetic construct
  REGION 1..18: note = Description of Artificial Sequence: Syntheticpeptide
  MOD_RES 9: note = D-amino acid
VLTKVKTVPPTKVEVKVL

SEQ ID NO: 22 - protein, 28 aa, synthetic construct
  REGION 1..28: note = Description of Artificial Sequence: Syntheticpeptide
  MOD_RES 10: note = D-amino acid
VEVSVSVEVPPTEVSVEVEVGGGGRGDV

SEQ ID NO: 23 - protein, 20 aa, synthetic construct
  REGION 1..20: note = Description of Artificial Sequence: Syntheticpeptide
  MOD_RES 10: note = D-amino acid
VEVSVSVEVPPTEVSVEVEV

(19) United States
(12) Patent Application Publication
(10) Pub. No.: US 2025/0134946 A1
(43) Pub. Date: May 1, 2025

(54) SELF-ASSEMBLING AMPHIPHILIC PEPTIDE HYDROGELS FOR TREATMENT OF NERVE INJURY

(71) Applicant: GEL4MED, INC., Bringhton, MA (US)

(72) Inventors: Manav Mehta, Newton, MA (US); Ravi Kiran Mekala, Bringhton, MA (US)

(21) Appl. No.: 18/682,493

(22) PCT Filed: Aug. 9, 2022
(86) PCT No.: PCT/US2022/074717

§ 371 Date: Feb. 9, 2024

Related U.S. Application Data
(60) Provisional application No. 63/231,153, filed on Aug. 9, 2021.

(51) Int. Cl. A61K 38/02; A61K 35/30; A61K 38/18; A61P 25/02

(52) U.S. Cl. CPC A61K 38/02; A61K 35/30; A61K 38/1866; A61P 25/02

(57) ABSTRACT

Methods of treating a nerve injury are disclosed. The methods include administering to a target site of the nerve injury a thermally stable preparation having a purified amphiphilic peptide in an aqueous biocompatible solution, being configured to self-assemble into a hydrogel, and administering to the target site a buffer having an effective amount of an ionic salt and a biological buffering agent to form the hydrogel. The methods include administering to the target site a biological material suspension in an amount effective to treat the nerve injury. The methods include administering to the target site an anti-scarring agent in an amount effective to treat the nerve injury. The target site is associated with central nervous system tissue or peripheral nervous system tissue. The nerve injury includes spinal cord injury and peripheral nerve injury.

### CROSS-REFERENCE TO RELATED APPLICATIONS

This application claims priority under 35 U.S.C. § 119(e) to U.S. Provisional Application Ser. No. 63/231,153, titled “SELF-ASSEMBLING AMPHIPHILIC PEPTIDE HYDROGELS FOR TREATMENT OF NERVE INJURY,” filed on Aug. 9, 2021, which is incorporated herein by reference in its entirety for all purposes.

### SEQUENCE LISTING

The instant application contains a Sequence Listing which has been submitted electronically in XML (extensible Markup Language) and is hereby incorporated by reference in its entirety. Said XML copy, created on Aug. 9, 2022, is named G2093-700600_SL.xml.

### FIELD OF TECHNOLOGY

Aspects and embodiments disclosed herein are directed toward systems and methods for treatment of nerve injury, such as spinal cord injury and peripheral nerve injury. In particular, aspects and embodiments are directed toward self-assembling amphiphilic peptide formulations and methods for the treatment of nerve injury.

### BACKGROUND

Nerve damage can be caused by a number of different factors. Certain types of nerve damage become increasingly common with age. Other types of nerve damage are associated with another indication, such as diabetes, autoimmune diseases, and cancer. Yet other types of nerve damage are caused by traumatic injury. Nerve damage has historically been difficult to treat. Often, the first goal of treatment is to address the underlying condition. However, in many instances there is no treatable underlying condition, such as for nerve damage caused by traumatic injury. There is a need for improved treatment of nerve damage.

Nerve damage may occur in the central nervous system (CNS). A lack of axonal regeneration in the injured or diseased adult mammalian CNS typically results in permanent functional impairment. The failure of CNS axons to regenerate has been associated with a nonpermissive nature of the glial cell environment surrounding the injury site or area of lost or damaged tissue.

Spinal cord injury (SCI) affects hundreds of thousands of people each year, placing a significant burden and cost on healthcare. SCI is a trauma of the CNS system and affects axonal regeneration in the CNS. Most SCI cases result from preventable causes such as injury during combat, road accidents, falls, or violence. SCI significantly reduces quality of life and lifespan of affected individuals. Life-long complications of SCI account for strain on families and are associated with depression and reduced ability to reintegrate into daily life. SCI patients and their families will continue to struggle unless new therapies can be developed that restore function and quality of life.

Peripheral nerves are fragile and easily damaged. Peripheral nerve injury (PNI) generally includes any damage to peripheral nerve axons and/or surrounding tissue. PNI cases result from traumatic injury, such as an accident, fall, or sports injury, medical conditions, such as diabetes, Guillain-Barre syndrome, and carpal tunnel syndrome, or autoimmune diseases, such as lupus, rheumatoid arthritis, and Sjogren's syndrome. Symptoms of PNI can range from mild discomfort to a serious limitation of daily activities. Symptoms generally depend on the type of nerve fiber affected, for example, motor nerves, sensory nerves, or autonomic nerves. PNI should be diagnosed and treated early to prevent complications and permanent damage. Effective and minimally invasive therapies are needed for rapid treatment of early onset PNI.

Cell therapy is the administration of cell-derived and tissue-derived material into a subject. Often, the cell and tissue-derived materials include biological fluids and/or living cells and tissues. Cell therapy is recognized as an important field in the treatment of disease. There is a need for materials capable of delivering living cells and cell and tissue-derived material to a target tissue site in a safe and effective manner.

### SUMMARY

In accordance with one aspect, there is provided a method of treating a nerve injury. The method may comprise administering to the target site a thermally stable preparation comprising a purified amphiphilic peptide in an aqueous biocompatible solution. The peptide may comprise a folding group having a plurality of charged amino acid residues and hydrophobic amino acid residues arranged in a substantially alternating pattern and a turn sequence. The peptide may be configured to self-assemble into a hydrogel. The method may comprise administering to the target site a buffer comprising an effective amount of an ionic salt and a biological buffering agent to form the hydrogel.

In accordance with another aspect, there is provided a method of treating a nerve injury. The method may comprise administering to the target site of the nerve injury a biological material suspension in an amount effective to treat the nerve injury. The method may comprise administering to the target site an anti-scarring agent in an amount effective to treat the nerve injury. The method may comprise administering to the target site of the nerve injury a thermally stable preparation comprising a purified amphiphilic peptide in an aqueous biocompatible solution. The peptide may comprise a folding group having a plurality of charged amino acid residues and hydrophobic amino acid residues arranged in a substantially alternating pattern and a turn sequence. The peptide may be configured to self-assemble into a hydrogel. The method may comprise administering to the target site a buffer comprising an effective amount of an ionic salt and a biological buffering agent to form the hydrogel.

The target site of the nerve injury may be associated with a central nervous system (CNS) tissue or a peripheral nervous system (PNS) tissue.

In some embodiments, the nerve injury may be a spinal cord injury (SCI).

In some embodiments, the nerve injury may be peripheral nerve injury (PNI).

The method may further comprise administering to the target site a biological material suspension, in an amount effective to treat the nerve injury.

The preparation may be administered in response to an SCI symptom or trigger.

The preparation may be administered in response to a PNI symptom or trigger.

The biological material may comprise at least one of cells, cell-derived material, tissue, and tissue derived material.

The cells, cell-derived material, tissue, and/or tissue derived material may be autologous, allogeneic, or xenogeneic.

The method may further comprise obtaining the cells, cell-derived material, tissue, and/or tissue derived material from a donor.

The method may further comprise obtaining the cells, cell-derived material, tissue, and/or tissue derived material from the subject.

In some embodiments, the cells may comprise at least one of stem cells and glial cells.

The stem cells may comprise at least one of bone marrow derived stromal cells and adipose derived stromal cells.

The stem cells may comprise at least one of embryonic stem cells and adult stem cells.

In some embodiments, the glial cells may comprise at least one of oligodendrocytes, astrocytes, ependymal cells, microglia, Schwann cells, and satellite cells.

The method may comprise administering an effective amount of an anti-scarring agent.

The anti-scarring agent may comprise at least one of receptor protein tyrosine phosphatase σ (RPTPσ) inhibitory peptide (ISP), chondroitinase ABC (ChaseABC), and polysialyl transferase (PST).

The method may comprise administering an effective amount of an axon regeneration agent.

The axon regeneration agent may comprise at least one of nerve growth factor (NGF), brain-derived neurotrophic factor (BDNF), glial cell line-derived neurotrophic factor (GDNF), vascular endothelial growth factor (VEGF), calcium, neurotrophin-3, interleukin-1 (IL-1), neuregulin, growth associated protein 43 (GAP-43), tubulin, actin, and heat-shock protein-27 (HSP-27).

The subject may be in need of treatment for the nerve injury or the subject may have been diagnosed with the nerve injury. The nerve injury may be mild. The nerve injury may be moderate. The nerve injury may be severe. The nerve injury may be acute. The nerve injury may be sub-acute. The nerve injury may be chronic.

In some embodiments, the amount and/or frequency of administration may be sufficient to promote treatment of the nerve injury. For example, the amount and/or frequency of administration may be sufficient to promote treatment of the PNI or SCI.

In some embodiments, the amount and/or frequency of administration may be sufficient to promote axon growth, axon migration, axon proliferation, axon alignment, axon regeneration, axon re-innervation, and/or axon attachment at the target site.

In some embodiments, the amount and/or frequency of administration may be sufficient to form a scaffold and promote at least one of cell attachment and cell migration from the target site to a site of migration.

In some embodiments, the amount and/or frequency of administration may be sufficient to promote bridging or void filling the target site of the nerve injury, and/or reducing or preventing scar formation at the target site of the nerve injury.

The method may comprise administering the biological material suspension, the anti-scarring agent, the preparation, and/or the buffer topically, parenterally, or enterally.

Parenteral administration may comprise administration to the target site by injection or by infusion.

In some embodiments, the biological material suspension, the anti-scarring agent, the preparation, and/or the buffer may be injected via a minimally invasive procedure selected from intravenous, intrasecal, intramuscular, subcutaneous, intradermal, intramedullary, intravascular, intraventricular, intrabiliary, intrathecal, or epidural administration.

The biological material suspension, the anti-scarring agent, the preparation, and/or the buffer may be administered topically to the target site by spray, dropper, film, squeeze tube, or syringe.

The method may comprise combining two or more of the biological material suspension, the anti-scarring agent, the preparation, and the buffer prior to administration.

The method may comprise combining the two or more of the biological material suspension, the anti-scarring agent, the preparation, and the buffer, less than about 1 minute, less than about 2 minutes, less than about 5 minutes, or less than about 10 minutes prior to administration.

The method may comprise combining the two or more of the biological material suspension, the anti-scarring agent, the preparation, and the buffer, at a point of use.

In some embodiments, the biological material suspension, the anti-scarring agent, the preparation, and the buffer may be administered separately.

The method may comprise combining an axon regeneration agent with one or more of the biological material suspension, the preparation, and the buffer prior to administration.

In some embodiments, the target site may be a tissue selected from mesenchymal tissue, connective tissue, muscle tissue, nervous tissue, embryonic tissue, dermal tissue, bone tissue, dental tissue, corneal tissue, cutaneous tissue, integumental tissue, soft tissue, hard tissue, and combinations thereof.

The target site may be associated with a desired local effect.

The peptide may comprise an effective amount of counterions.

The peptide may comprise an effective amount of acetate, citrate, and/or chloride counterions.

In some embodiments, the peptide may be substantially free of chloride counterions.

The buffer may comprise between about 10 mM and 150 mM sodium chloride and between about 10 mM and 100 mM Bis-tris propane (BTP).

The method may comprise administering the biological material suspension, the anti-scarring agent, the preparation, and/or the buffer in combination with a surgical procedure.

The method may comprise administering a first dosage of the biological material suspension, the anti-scarring agent, the preparation, and/or the buffer.

The method may comprise administering at least one booster dosage of the biological material suspension, the anti-scarring agent, the preparation, and/or the buffer.

The hydrophobic amino acid residues may be independently selected from glycine, alanine, valine, leucine, isoleucine, proline, phenylalanine, methionine, threonine, tryptophan, and combinations thereof.

The charged amino acid residues may be independently selected from arginine, lysine, histidine, and combinations thereof.

The folding group may have a sequence comprising Y[XY]_{N}[T][YX]_{M}Y, where X is 1-3 charged amino acids, Y is 1-3 hydrophobic amino acids, T is 2-8 turn sequence amino acids, and N and M are each independently between 2 and 10.

The turn sequence amino acids may be independently selected from a D-proline, an L-proline, aspartic acid, threonine, asparagine, and combinations thereof.

The peptide may be configured to self-assemble into a substantially biocompatible hydrogel.

The peptide may be configured to self-assemble into a hydrogel having at least one property selected from a cell friendly hydrogel, a substantially biodegradable, non-inflammatory, and/or non-toxic hydrogel, a hydrogel having substantially low hemolytic activity, and a hydrogel having substantially low immunogenic activity.

The method may further comprise administering at least one combination treatment selected from: an antibacterial composition, an antifungal composition, an antiviral composition, an anti-tumor composition, an anti-inflammatory composition, a cell culture media, a cell culture serum, an anti-odor composition, a hemostatic composition, and an analgesic or pain-relief composition.

The combination treatment may be administered prior to the preparation.

The combination treatment may be administered after the preparation.

The combination treatment may be administered concurrently with the preparation.

The peptide may be at least 80% purified, for example, at least 85%, at least 90%, at least 92%, at least 95%, at least 98%, at least 99%, or at least 99.9%.

The purified peptide may have less than 10% residual organic solvent by weight, for example, less than 8%, less than 5%, less than 2%, less than 1%, or less than 0.1%.

The organic solvent may comprise at least one of trifluoroacetic acid (TFA), acetonitrile, isopropanol, N,N-Dimethylformamide, triethylamine, Ethyl Ether, and acetic acid.

The preparation may have a residual Trifluoroacetic acid (TFA) concentration of less than about 1% w/v, a residual acetonitrile concentration of less than about 410 ppm, a residual N,N-Dimethylformamide concentration of less than about 880 ppm, a residual triethylamine concentration of less than about 5000 ppm, a residual Ethyl Ether concentration of less than about 1000 ppm, a residual isopropanol concentration of less than about 100 ppm, and/or a residual acetic acid concentration of less than 0.1% w/v.

The peptide may include a functional group.

The functional group may have between 3 and 30 amino acid residues.

The functional group may be engineered to express a bioactive property.

The functional group may be engineered to control or alter charge of the peptide or preparation.

The functional group may be engineered to control or alter pH of the peptide or preparation.

The functional group may be engineered for a target indication.

The target indication may be selected from cell culture, cell delivery, wound healing, treatment of biofilm, and combinations thereof.

The functional group may have a sequence selected from RGD, IKVAV, YIGSR, LKKTETQ, SNKPGVL, PKPQQFFGLM, GKLTWQELYQLKYKGI, and GGG.

The peptide may be configured to self-assemble into a substantially ionically-crosslinked hydrogel.

The peptide may be configured to self-assemble into a shear-thinning hydrogel.

The peptide may be configured to self-assemble into a substantially transparent hydrogel.

The buffer may comprise from about 5 mM to about 200 mM ionic salts.

The ionic salts may dissociate into at least one of sodium, potassium, calcium, magnesium, iron, ammonium, pyridium, quaternary ammonium, chloride, and sulfate ions.

The ionic salts may comprise sodium chloride, ammonium chloride, magnesium chloride, potassium chloride, calcium chloride, ammonium sulfate, magnesium sulfate, sodium sulfate, potassium sulfate, calcium sulfate, sodium bicarbonate, and combinations thereof.

The buffer may comprise from about 10 mM to about 150 mM sodium chloride.

The peptide may have a bacterial endotoxin level of less than about 10 EU/mg.

The preparation may comprise between 0.1% w/v and 8.0% w/v of the peptide.

The preparation may comprise between 0.5% w/v and 6.0% w/v of the peptide.

The preparation may comprise between 0.5% w/v and 3.0% w/v of the peptide.

The preparation may comprise between 0.5% w/v and 1.5% w/v of the peptide.

The preparation may comprise between 0.5% w/v and 1.0% w/v of the peptide.

The preparation may comprise between 0.7% w/v and 2.0% w/v of the peptide.

The preparation may comprise between 0.7% w/v and 0.8% w/v of the peptide.

The hydrogel may comprise between 0.25% w/v and 6.0% w/v of the peptide.

The hydrogel may comprise between 1.5% w/v and 6.0% w/v of the peptide.

The hydrogel may comprise between 0.25% w/v and 3.0% w/v of the peptide.

The peptide may be configured to self-assemble into a hydrogel having between 90% w/v and 99.9% w/v aqueous solution.

The peptide may have a net charge of from −7 to +11.

The peptide may have a net charge of from +2 to +9.

The peptide may have a net charge of from +5 to +9.

The peptide may be lyophilized.

The preparation may be sterile.

The preparation may be substantially free of a preservative.

The preparation may be thermally stable between −20° C. and 150° C.

The preparation may be sterilized by or autoclave sterilization.

The method may comprise providing at least one of the biological material suspension, the anti-scarring agent, the preparation, and the buffer. For instance, the method may comprise providing the biological material suspension. The method may comprise providing the anti-scarring agent. The method may comprise providing the preparation. The method may comprise providing the buffer.

The method may comprise providing at least one of the peptide and the biocompatible solution. For instance, the method may comprise providing the peptide. The method may comprise providing the biocompatible solution.

The method may comprise providing at least one of the biological material suspension, the anti-scarring agent, the peptide, the biocompatible solution, and the buffer separately.

In accordance with another aspect, there is provided a method of preparing a nerve injury treatment composition. The method may comprise combining a therapeutically effective amount of a biological material suspension with a preparation comprising a purified amphiphilic peptide comprising a folding group having a plurality of charged amino acid residues and hydrophobic amino acid residues arranged in a substantially alternating pattern and a turn sequence, the peptide being configured to self-assemble into a hydrogel, and a buffer comprising an effective amount of an ionic salt and a biological buffering agent to form the hydrogel.

The method may comprise combining the biological material suspension with the preparation to produce a biological material peptide preparation. The method may comprise combining the biological material peptide preparation with the buffer to form the hydrogel.

The method may comprise combining the biological material suspension with the buffer to produce a biological material buffer suspension. The method may comprise combining the biological material buffer suspension with the preparation to form the hydrogel.

The method may comprise combining the preparation with the buffer to form the hydrogel. The method may comprise combining the biological material suspension with the hydrogel to produce the nerve injury treatment composition.

The method may comprise combining the biological material suspension with an anti-scarring agent.

The method may comprise combining at least two of the biological material suspension, the anti-scarring agent, the preparation, and the buffer in vitro.

The method may comprise combining at least two of the biological material suspension, the anti-scarring agent, the preparation, and the buffer in vivo.

The method may comprise combining at least two of the biological material suspension, the anti-scarring agent, the preparation, and the buffer in situ.

The method may comprise combining the preparation with the buffer to form the hydrogel in vitro, and combining the biological material suspension with the hydrogel in vivo.

The biological material suspension may comprise at least one of cells, cell-derived materials, tissue, and tissue-derived materials. The method may comprise culturing the biological material in the hydrogel for a predetermined period of time prior to administration to a subject.

In some embodiments, the hydrogel may comprise a non-homogeneous suspension of the cells, e.g., comprising clusters or spheroids of the cells.

The method may comprise combining the biological material suspension with a cell culture media, cell maintenance agent, cell growth agent, cell culture serum, or combination thereof.

In accordance with another aspect, there is provided a method of facilitating treatment of a nerve injury in a subject. The method may comprise providing a preparation comprising a purified amphiphilic peptide comprising a folding group having a plurality of charged amino acid residues and hydrophobic amino acid residues arranged in a substantially alternating pattern and a turn sequence, the peptide being configured to self-assemble into a hydrogel. The method may comprise providing instructions to combine biological material with the preparation and a buffer comprising an effective amount of an ionic salt and a biological buffering agent to form the hydrogel. The method may comprise providing instructions to agitate the hydrogel comprising the biological material to produce a biological material suspension hydrogel. The method may comprise providing instructions to administer an effective amount of the non-homogeneous glial cell suspension hydrogel to a target site of the nerve injury to provide treatment of the nerve injury to the subject.

In some embodiments, the method may comprise providing the buffer.

In some embodiments, the method may comprise providing the biological material.

The biological material may comprise at least one of cells, cell-derived materials, tissue, and tissue-derived materials.

In some embodiments, administering the effective amount of the biological material suspension hydrogel to the target site provides treatment of spinal cord injury (SCI).

In some embodiments, administering the effective amount of the biological material suspension hydrogel to the target site provides treatment of peripheral nerve injury (PNI).

The method may comprise providing at least one of a mixing device configured to agitate the hydrogel and a delivery device configured to administer the biological material suspension hydrogel.

In accordance with yet another aspect, there is provided a kit. The kit may comprise a biological material suspension. The kit may comprise a preparation comprising a purified amphiphilic peptide comprising a folding group having a plurality of charged amino acid residues and hydrophobic amino acid residues arranged in a substantially alternating pattern and a turn sequence, the peptide being configured to self-assemble into a hydrogel. The kit may comprise a buffer configured to induce self-assembly of the peptide into the hydrogel. The kit may comprise instructions to combine the biological material suspension, the preparation, and the buffer prior to or concurrently with administration of the preparation to a subject.

In some embodiments, the kit may further comprise an anti-scarring agent.

The disclosure contemplates all combinations of any one or more of the foregoing aspects and/or embodiments, as well as combinations with any one or more of the embodiments set forth in the detailed description and any examples.

### BRIEF DESCRIPTION OF DRAWINGS

The accompanying drawings are not intended to be drawn to scale. In the drawings, each identical or nearly identical component that is illustrated in various figures is represented by a like numeral. For purposes of clarity, not every component may be labeled in every drawing. In the drawings:

FIG. 1A includes schematic and microscopic images of an assembled peptide hydrogel matrix with encapsulated cells as compared to collagen, according to one embodiment;

FIG. 1B includes a schematic drawing of a mixing device and a schematic representation of cells in a hydrogel matrix, according to one embodiment;

FIG. 2 includes images of sustained therapeutic activity of the administered peptide hydrogel compared to conventional polymer, according to one embodiment;

FIG. 3 is a microscopy image of positively charged peptide hydrogels, according to one embodiment;

FIG. 4 is a graph showing the antimicrobial activity of the peptide hydrogel, in accordance with one embodiment;

FIG. 5 includes images of a mouse model post-burn injury with bacterial infection showing antimicrobial activity of the peptide hydrogel, in accordance with one embodiment;

FIG. 6A includes graphs showing controlled release of therapeutics from peptide hydrogels, according to one embodiment;

FIG. 6B shown an image of the SDS page test related to the graphs of FIG. 2, according to one embodiment;

FIG. 7A is a graph of static light scattering (SLS) at 266 nm of exemplary peptides as a function of temperature, according to some embodiments;

FIG. 7B is a graph of static light scattering (SLS) at 266 nm of exemplary peptides as a function of temperature, according to some embodiments;

FIG. 8 includes graphs showing absorbance of a peptide hydrogel as a function of peptide concentration, according to one embodiment;

FIG. 9 is a graph showing net charge of a peptide preparation as a function of pH value, according to one embodiment;

FIG. 10 is a visual representation of net peptide charge at a pH of 7.4 for several amino acid residues, according to one embodiment;

FIG. 11 includes schematic drawings showing methods of incorporating biological material in a hydrogel, according to one embodiment;

FIG. 12 includes a schematic drawing of a mixing device, images showing cell distribution in the hydrogel after use of the mixing device, and graphs showing results from a luciferase assay demonstrating viability, according to one embodiment;

FIG. 13A is a schematic diagram of a method for biofabricating hydrogels, according to one embodiment;

FIG. 13B is a schematic diagram of a method for biofabricating hydrogels, according to one embodiment;

FIG. 13C is a schematic diagram of a method for biofabricating hydrogels, according to one embodiment;

FIG. 14 includes a schematic drawing and bright-field microscopy images showing cells seeded on top of a hydrogel and graphs showing results from a luciferase viability assay, according to one embodiment;

FIG. 15 includes images of polyacrylamide gel electrophoresis (PAGE) showing release of encapsulated enzyme from hydrogels with varying peptide concentrations, according to one embodiment;

FIG. 16 is a graph showing cell viability in hydrogels of varying pH value, according to one embodiment;

FIG. 17 is a line drawing of a photograph of the preparation provided in an end-use container, according to one embodiment;

FIG. 18 includes line drawings of photographs of highly concentrated cells in self-assembling peptide hydrogels and graphs of luciferase viability assays, according to one embodiment;

FIG. 19 includes graphs from luciferase cell viability assays and microscopy images of the hydrogel, according to one embodiment;

FIG. 20 includes images of methicillin-resistant Staphylococcus aureus (MRSA, ATCC 33591) (n=6) (A-C) or Pseudomonas aeruginosa (PA01) cultured on agar plates with the self-assembling peptide hydrogel and graphs showing antimicrobial efficacy of the hydrogel, according to one embodiment;

FIG. 21 includes a graph showing viability and proliferation of Schwann Cells cultured on a peptide hydrogel and fluorescence microscopy images of the cultured Schwann cells, according to one embodiment; and

FIG. 22 includes images demonstrating hydrogel repair of peripheral nerve injury in a rodent model, according to one embodiment.

### DETAILED DESCRIPTION

Methods of treatment of spinal cord injury (SCI) and peripheral nerve injury (PNI) are disclosed herein. The methods may generally comprise administering a preparation comprising a self-assembling peptide to a target site of the injury. The self-assembling peptide may be configured to assemble into a hydrogel having controlled and/or selected properties. In certain embodiments, the hydrogel may form a matrix for cells or other biological material to be administered to the target site. Therapeutic methods of cell transplantation may provide the injured spinal cord or peripheral nerve with support of axon regeneration. In certain instances, cell transplantation may reconnect and remyelinate, as well as provide protection and replacement, for lost neural tissue. Thus, the preparations disclosed herein may be employed to provide cell and biological material transplantation to a site of an injury for treatment therein.

Preparations comprising self-assembling peptide hydrogels are disclosed herein. The self-assembled peptide may be amphiphilic. The peptide may generally have a folding group having a plurality of charged amino acid residues and hydrophobic residues arranged in a substantially alternating pattern. The peptide may include functional groups to provide desired physical or chemical properties upon administration. The purified peptide may include counterions that improve biocompatibility of the preparation. The counterions may control the self-assembly, physical and chemical properties of the peptide. The counterions may enhance the therapeutic functional properties of the peptide. The preparation may include the peptide in an aqueous biocompatible solution. The preparation may include a buffer solution capable of inducing self-assembly of the peptide upon contact. The buffer solution may contain a buffering agent and ionic salts. The buffer solution composition may be designed to control the assembled hydrogel's physical or chemical properties. The preparation may be designed to be thermally stable.

In general, the preparation may have shear-thinning properties and a substantially physiological pH level. The self-assembled hydrogel may have antimicrobial, antiviral, and/or antifungal properties. The preparation may be administered topically or parenterally. The preparation may be administered for tissue engineering applications. Certain exemplary applications include cell delivery, cell culture, treatment and prevention of fungal infections, treatment and prevention of bacterial infections, wound healing, biofilm treatment, biofilm management, and prevention of biofilm and wound infection, including infection of chronic wounds. Other tissue engineering applications are within the scope of the disclosure.

Methods of administering the preparation to a subject are disclosed herein. The methods may generally include selecting a target site for administration and administering the preparation to the target site. Methods of administering the preparation may also include mixing the preparation with a buffer configured to induce self-assembly of the peptide to form the hydrogel and administering the hydrogel to the target site. In certain exemplary embodiments, the preparation and/or hydrogel may be administered by spray, aerosol, dropper, tube, ampule, instillation, injection, or syringe.

In certain embodiments, methods of administering cells to a subject are disclosed herein. The methods may generally include suspending the cells in a solution comprising a self-assembling peptide and administering an effective amount of the suspension to a target site of the subject. The methods may comprise combining the solution with a buffer configured to induce self-assembly of the peptide. The solution may be combined with the buffer prior to administration, concurrently with administration, or after administration. The buffer may generally comprise an effective amount of an ionic salt and a biological buffering agent.

Unlike other peptides in aqueous solution, the peptides disclosed herein undergo self-assembly. The self-assembly may enable the peptides to be administered in a concentrated or localized manner to a target tissue. For example, self-assembling peptides may be administered at higher concentrations when compared to free floating peptides. The self-assembling peptides may exhibit the clinical benefit of reducing offsite toxicity of the peptides, due to the localizing effect upon administration. Additionally, the therapeutic dosage of peptides may be increased in the vicinity of the target administration site.

Unlike other polymers in aqueous solution, the peptides disclosed herein may undergo self-assembly in situ at the target site. The in situ self-assembly may enable the peptides to be administered to a target tissue and allow to physically or ionically crosslink, for example, within seconds of administration. For example, self-assembling peptides may be administered directly to target site. Conventional free-floating peptides or polymers usually need a crosslinking agent or exogenous added covalent crosslinking agent. Thus, the self-assembling peptides disclosed herein may provide the clinical benefit of reducing product application and complexity. Additionally, the ionic crosslinking of peptides upon self-assembly may provide the benefit of selecting between product removal and permanent adherence to a target administration site.

### Select Definitions

Hydrogels are a class of materials that have significant promise for use in soft tissue and bone engineering. The general characteristic of hydrogels that make them important materials for these applications are their well hydrated, porous structure. Hydrogels may be designed to be compatible with the adhesion and proliferation of various cell types, e.g., fibroblasts and osteoblasts, making them potential tissue engineering scaffolds for generating connective tissue, such as cartilage, tendons, and ligaments, and bone.

The hydrogel material may be cytocompatible. Cytocompatibility, defined herein, means that the hydrogel must not be adverse to desired cells, in vitro and/or in vivo. Adversity to cells may be measured by cytotoxicity, cell adhesion, proliferation, phenotype maintenance, and/or differentiation of progenitor cells.

The hydrogel material may be biocompatible. “Biocompatible,” defined herein, means that a material does not cause a significant immunological and/or inflammatory response if placed in vivo. Biocompatibility may be measured according to International Organization for Standardization (ISO) 10993 standards.

The hydrogel material may be biodegradable affording non-toxic species. The hydrogel material may be proteolytically biodegradable. “Proteolytic” biodegradation, defined herein, refers to local degradation of the material in response to the presence of cell-derived proteases and/or gradual degradation with the proliferation of cells. The hydrogel material may be hydrolytically biodegradable. “Hydrolytic” biodegradation, defined herein, refers to polymer degradation without assistance from enzyme under biologic conditions.

The hydrogel material may be bioresorbable. Bioresorbable, defined herein, means that the hydrogel material breaks down into remnants that are natural products readily absorbed into the body, resulting in complete loss of original mass.

The hydrogel material may be shear-thinning. “Shear-thinning,” as described herein, refers to a variable apparent viscosity, in particular, a decreasing viscosity with increasing applied stress. For instance, the shear-thinning hydrogel may exhibit non-Newtonian fluid properties. In particular the hydrogels disclosed herein may be administered through a needle or catheter and rapidly resume gelation after removal of the mechanical force.

The hydrogel and/or other materials disclosed herein may be referred to as having one or more physiological properties. As disclosed herein, physiological properties or values refer to those which are compatible with the subject. In particular, physiological properties or values may refer to those which are compatible with a particular target tissue. In certain embodiments, physiological properties or values may refer to those which are substantially similar to the properties or values of the target tissue. Physiological properties may include one or more of pH value, temperature, net charge, water content, stiffness, and others.

“Self-assembling” peptides include such peptides which, typically, after being exposed to a stimulus, will assume a desired secondary structure. The peptides may self-assemble into a higher order structure, for example a three-dimensional network and, consequently, a hydrogel. The self-assembled hydrogel may contain peptides in a tertiary and/or quaternary structure through charge screening, hydrophobic, and disulfide interactions. Peptides have been observed to self-assemble into helical ribbons, nanofibers, nanotubes and vesicles, surface-assembled structures and others. Self-assembling peptides may assemble responsive to certain environmental conditions, e.g., pH, temperature, net charge, exposure to light, applied sound wave, or presence or absence of environmental factors. The environmental conditions may occur upon administration to a subject or by combination with a buffer. In other embodiments, the peptide may assemble spontaneously in solution under neutral pH level. The peptide may assemble spontaneously in solution under physiological conditions and/or in the presence of a cation and/or anion.

The self-assembling peptides may assemble into an alpha helix, pi-helix, beta sheet, random coil, turn, beta pleated parallel, antiparallel, twist, bulge, or strand connection secondary structure and combinations of thereof. For example, a 20 amino acid peptide which self-assembles into β-strands may comprise alternating valine and lysine residues flanking a tetrapeptide sequence (-VDPPT-). When dissolved in low ionic strength and buffered aqueous solution, the exemplary peptide resides in an ensemble of random coil conformers due to electrostatic repulsions of the positively charged lysine residues. Upon increasing the ionic strength and/or pH of the solution, the lysine-based positive charge is relieved due to either screening of the charge or deprotonating a sufficient amount of the side chain amines. This exemplary action enables peptide folding into an amphiphilic β-hairpin. In the folded state, the exemplary peptide self-assembles via lateral and facial associations of the hairpins to form a non-covalently crosslinked hydrogel containing β-sheet rich fibrils. Thus, the self-assembling peptides may be designed to undergo hydrogelation under varying conditions through rational design of the peptide sequence.

The self-assembling peptides disclosed herein may assemble into a nano-porous tertiary structure. As disclosed herein, the nano-porous structure is a three-dimensional matrix containing pores having an average size of 1-1000 nm. The pores or voids may constitute between 10% and 90% of the three-dimensional matrix by volume. For example, the pores or voids may constitute 10%, 20%, 30%, 40%, 50%, 60%, 70%, 80%, or 90% of the three-dimensional matrix by volume. The pores may be permeable and allow diffusion of liquid and/or gas. The nano-porous structure is constructed by physical crosslinks, allowing ionic bonds to be broken and reformed upon asserted stress. These nano-porous structure may allow for cells to attach and/or migrate through the matrix. The nano-porous structure may also mimic the endogenous extra-cellular matrix environment of tissues and, optionally, be selected to mimic a specific tissue.

“Disassembly” of the peptides may refer to the ability of the peptide to assume a lower order structure after being exposed to a stimulus. Disassembly may also refer to the ability of the physically crosslinked peptide to temporarily break hydrophobic and disulfide bonds to assume a lower order structure after being exposed to a stimulus. For example, a tertiary structure protein may disassemble into a secondary structure protein, and further disassemble into a primary structure peptide. In accordance with certain embodiments, self-assembly and disassembly of the peptide may be reversible.

Preparations and formulations disclosed herein may generally be referred to as peptide preparations. The peptide preparations may include a self-assembling peptide and/or a self-assembled hydrogel as disclosed herein. The peptide preparation may include a cytocompatible and/or biocompatible solution. The preparation may include a buffer. While reference is made to a solution, it should be understood that the preparation may be in the form of a liquid, gel, or solid particle. In certain embodiments, for example, the preparation may be in the form of the assembled hydrogel. In other embodiments, the preparation may be in the form of a lyophilized powder.

The peptide preparation may further include one or more bioactive components for tissue engineering, such as, functionalized peptides, cells, media, serum, collagen and other structure-imparting components, antibodies and antigens, bioactive small molecules, and other bioactive drugs. “Bioactivity” as described herein refers to the ability of a compound to impart a biological effect.

Cell containing preparations and formulations disclosed herein may be referred to as cell suspensions. Cell suspensions include a plurality of cells, e.g., living cells, suspended in a solution. The solution may be or comprise water, media, or buffer. The suspension may generally further comprise a self-assembling peptide and/or a self-assembled hydrogel, as disclosed herein. While reference is made to cells, it should be understood that the suspension may contain cell fragments and/or tissue, e.g. tissue grafts, in addition to or instead of the cells. For example, the suspension may contain live or dead cells or cell fragments, spheroids, and/or cell aggregates.

The cells may be isolated from living tissue and subsequently maintained and/or grown in cell culture. The cell culture conditions may vary, but generally include maintaining the cells in a suitable vessel with a substrate or medium that supplies the essential nutrients, e.g., amino acids, carbohydrates, vitamins, minerals, growth factors, hormones, and gases, e.g., CO₂ and O₂, and regulating the physio-chemical environment, e.g., pH, osmotic pressure, temperature. The cells may be maintained in live cell lines, e.g., a population of HeLa cells descended from a single cell and containing the same genetic makeup.

The term “isolated,” as used herein, refers to material that is removed from its original or native environment (e.g., the natural environment if it is naturally occurring). For example, a naturally-occurring polynucleotide or polypeptide present in a living animal is not isolated, but the same polynucleotide or polypeptide, separated by human intervention from some or all of the co-existing materials in the natural system, is isolated. Such polynucleotides could be part of a vector and/or such polynucleotides or polypeptides could be part of a composition, and still be isolated in that such vector or composition is not part of the environment in which it is found in nature.

As used herein, “treatment” of an injury, condition, or disease refers to reducing the severity or frequency of at least one symptom of that injury, condition, or disease, compared to a similar but untreated subject. Treatment can also refer to halting, slowing, or reversing the progression of an injury, condition, or disease, compared to a similar but untreated subject. Treatment may comprise addressing the root cause of the injury, condition, or disease and/or one or more symptoms. “Management” of an injury, condition, or disease may refer to reducing the severity or frequency of at least one symptom of that injury, condition, or disease, to a tolerable level, as determined by the subject or a health care provider.

As used herein an effective amount refers to a dose sufficient to achieve a desired result. For example, the effective amount may refer to a concentration sufficient to achieve self-assembly of the hydrogel and/or provide desired properties. An effective amount may refer to a dose sufficient to prevent advancement, or to cause regression of an injury, condition, or disease, or which is capable of relieving a symptom of an injury, condition, or disease, or which is capable of achieving a desired result. An effective amount can be measured, for example, as a concentration of peptide or other component in the preparation, solution, or buffer. An effective amount can be measured, for example, as a concentration of bioactive agent or an effect or byproduct of a bioactive agent. An effective amount can be measured, for example, as a number of cells or number of viable cells, or a mass of cells (e.g., in milligrams, grams, or kilograms), or a volume of cells (e.g., in mm³).

Throughout this disclosure, formulation may refer to a composition or preparation or product.

Administered “in combination,” as used herein, means that two (or more) different treatments are delivered to the subject during the course of the subject's affliction with the injury, e.g., the preparation is delivered with a second agent after the subject has been diagnosed with the condition or injury and before the condition or injury has been cured or eliminated. In certain embodiments, administration in combination means the preparation additionally comprises one or more second agent. In some embodiments, the delivery of one treatment is still occurring when the delivery of the second begins, so that there is overlap. This is sometimes referred to herein as “simultaneous” or “concomitant” or “concurrent delivery.” In other embodiments, the delivery of one treatment ends before the delivery of the other treatment begins. This is sometimes referred to herein as “successive” or “sequential delivery.”

In embodiments of either case, the treatment is more effective because of combined administration. For example, the second agent is a more effective, e.g., an equivalent effect is seen with less of the second agent, or the second agent reduces symptoms to a greater extent, than would be seen if the second agent were administered in the absence of the preparations disclosed herein, or the analogous situation is seen with the preparation. In some embodiments, delivery is such that the reduction in a symptom, or other parameter related to the disorder is greater than what would be observed with one treatment delivered in the absence of the other. The effect of the two treatments can be partially additive, wholly additive, or greater than additive (i.e., synergistic). The delivery can be such that an effect of the administration of the preparation is still detectable when the second agent is delivered. In some embodiments, one or more treatment may be delivered prior to diagnosis of the patient with the injury.

As used herein, a subject may include an animal, a mammal, a human, a non-human animal, a livestock animal, or a companion animal. The term “subject” is intended to include human and non-human animals, for example, vertebrates, large animals, and primates. In certain embodiments, the subject is a mammalian subject, and in particular embodiments, the subject is a human subject. Although applications with humans are clearly foreseen, veterinary applications, for example, with non-human animals, are also envisaged herein. The term “non-human animals” of the disclosure includes all vertebrates, for example, non-mammals (such as birds, for example, chickens; amphibians; reptiles) and mammals, such as non-human primates, domesticated, and agriculturally useful animals, for example, sheep, dog, cat, cow, pig, rat, among others. The term “non-human animals” includes research animals, for example, for example, mouse, rat, rabbit, dog, cat, pig, among others.

### Properties of the Peptide Sequence and Secondary Structure

The peptides disclosed herein may have a sequence configured to fold into a desired secondary structure. The secondary structure may refer to a three-dimensional form of local segments of proteins. The secondary structure may comprise, for example, pleated sheet, helical ribbon, nanotube and vesicle, surface-assembled structure, and others. The peptides disclosed herein may have a sequence configured to self-assemble into a desired tertiary structure. The tertiary structure may refer to a three-dimensional organization of secondary structure protein forms. The tertiary structure may comprise, for example, three-dimensional matrix, porous matrix, nano-porous matrix.

Self-assembling peptides disclosed may be designed to adopt a secondary, for example, β-hairpin, and/or tertiary structure in response to one or more signals. Typically, after adopting the secondary structure, the peptides will self-assemble into a higher order structure, e.g., a hydrogel. In certain embodiments, the self-assembly does not take place unless side chains on the peptide molecules are uniquely presented in the secondary structure conformation. The self-assembling peptides may assemble responsive to certain environmental conditions, e.g., pH, temperature, net charge, exposure to light, applied sound wave, or presence or absence of environmental factors. The environmental conditions which induce self-assembly may occur upon administration to a subject, e.g., upon contact with a target tissue. In some embodiments, the environmental conditions which induce self-assembly may occur upon combination of the peptide preparation with a buffer configured to induce self-assembly. The buffer may have a pH or composition configured to induce self-assembly. For example, the buffer may have a concentration of ions configured to induce self-assembly.

Self-assembly of the peptides disclosed herein may produce compact structures that exhibit biophysical structural relationships with the intended function of the peptide. For example, a compact tertiary structure may have a higher number of active amino acid residues per unit area, compared to unassembled peptides. In the particular example of antimicrobial peptides, the tertiary structure may enable a higher concentration of charged, e.g., positively charged, amino acid residues per area, increasing antimicrobial properties (e.g., bacterial membrane destabilization and disruption).

In certain embodiments, the self-assembling peptide hydrogels may include those disclosed in and/or prepared by the methods disclosed in any of U.S. Pat. Nos. 8,221,773; 7,884,185; 8,426,559; 7,858,585; and 8,834,926, incorporated herein by reference in their entireties for all purposes. For example, the self-assembling peptide hydrogels may be or comprise any of SEQ ID NOS: 1-20 from U.S. Pat. Nos. 8,221,773, 7,884,185, and 7,858,585; and SEQ ID NOS: 1-33 from U.S. Pat. No. 8,834,926. Other self-assembling peptides are known and may be employed to bring about the methods disclosed herein.

The desired properties of the self-assembling peptides may be controlled by peptide design. The self-assembling peptides may be small peptides, e.g., from about 6 to about 200 residues or from about 6 to about 50 residues or from about 10 to about 50 residues. Any of the amino acid residues may be a D isoform. Any of the amino acid residues may be an L isoform.

Self-assembling peptides disclosed herein may be designed to be substantially amphiphilic when assembled into the tertiary structure. “Amphiphilic” molecules, e.g., macromolecules or polymers, as disclosed herein, typically contain hydrophobic and hydrophilic components. Peptide amphiphiles are one exemplary class of amphiphilic molecules. Peptide amphiphiles are peptide-based molecules that typically have the tendency to self-assemble into high-aspect-ratio nanostructures under certain conditions. The exemplary conditions may comprise selected pH, temperature, and ionic strength values. One particular type of peptide amphiphiles comprise alternating charged, neutral, and hydrophobic residues, in a repeated pattern, for example, as disclosed herein. A combination of intermolecular hydrogen bonding and hydrophobic and electrostatic interactions may be designed to form well-defined self-assembled nanostructures by assembly of the disclosed peptide amphiphiles.

The self-assembling peptides may include additional amino acids, for example, an epitope. For example, the self-assembling peptides may include additional functional groups, optionally selected by peptide design. Exemplary functional groups disclosed herein comprise a biologically derived motif, for example, having an effect on biological processes such as cell signal transduction, cell adhesion in the extra-cellular matrix (ECM), cell growth, and cell mobility. The peptide may include one or more modifications, for example, a linker or spacer.

In some embodiments, at least one of the N-terminus and the C-terminus may be modified. For example, at least one of the N-terminus and the C-terminus may be amidated. At least one of the N-terminus and the C-terminus may comprise a cysteine moiety. At least one of the N-terminus and the C-terminus may be acetylated. In certain exemplary embodiments, the C-terminus may be amidated and/or the N-terminus may be acetylated. In some embodiments, at least one of the N-terminus and the C-terminus may be free.

One exemplary amidated self-assembling peptide is CVKVRVRVRV(d)PPTRVRVRV KV-NH₂. Any of the peptides disclosed herein may be amidated. One exemplary acetylated and amidated self-assembling peptide is Ac-CVKVRVRVRV(d)PPTRVRVRVKVC-NH₂. Any of the peptides disclosed herein may be amidated, acetylated, or both.

In general, the self-assembling peptides may have a folding group configured to adopt the secondary and/or higher order structure. Exemplary self-assembling peptides may have a folding group designed to adopt a β-hairpin secondary structure. Exemplary self-assembling peptides may have a folding group designed to adopt a three-dimensional nano-porous matrix tertiary structure. Self-assembling peptides disclosed herein may be designed to adopt a β-hairpin secondary structure and/or nano-porous matrix tertiary structure in response to one or more environmental stimulus at the target site, e.g., at a topical or parenteral site. The self-assembling peptides may also be designed to self-assemble into a range of other self-assembled structures, such as spherical micelles, vesicles, bilayers (lamellar structures), nanofibers, nanotubes, and ribbons.

The self-assembly folding group may have between about 2 and about 200 residues, for example, between about 2 and about 50 residues, between about 10 and about 30 residues, between about 15 and about 25 residues, for example, about 20 residues.

In accordance with some embodiments, the self-assembling folding group may include hydrophobic amino acids. “Hydrophobic” amino acid residues are those which tend to repel water. Such hydrophobic amino acids may include glycine, alanine, valine, leucine, isoleucine, proline, phenylalanine, methionine, threonine, and tryptophan. In certain embodiments, the hydrophobic amino acid residues may comprise valine.

The folding group may be functionalized by addition of other functional residues as described herein, or conserved for self-assembly. Exemplary functional residues include basic, neutral, aliphatic, aromatic, and polar amino acid residues.

The folding group may have a plurality of basic, neutral, aliphatic, aromatic, polar, charged amino acid residues. The folding group may have a plurality of hydrophobic amino acid residues arranged in a substantially alternating pattern with non-hydrophobic amino acid residues. In certain embodiments, the folding group may have a plurality of hydrophobic amino acid residues arranged in a substantially alternating pattern with a plurality of charged amino acid residues.

The folding group may comprise a turn sequence. The turn sequence may include one or more internal amino acid residues within the folding group. In certain embodiments, the turn sequence may be substantially centrally located within the folding group.

The turn sequence may have between about 2 and about 20 residues, for example, between about 2 and about 10 residues, between about 2 and about 8 residues, between about 2 and about 5 residues, for example, about 2 residues, about 3 residues, about 4 residues, or about 5 residues.

In exemplary embodiments, the turn sequence may include one or more of proline, aspartic acid, threonine, and asparagine. The turn sequence may include D-proline and/or L-proline. In some embodiments, the turn sequence may have 1-4 proline residues, for example, 1 proline residue, 2 proline residues, 3 proline residues, or 4 proline residues.

Exemplary self-assembling peptides may have a folding group sequence comprising [AY]_{N}[T][YA]_{M}, where A is 1-3 amino acids selected from one or more of basic, neutral, aliphatic, aromatic, polar, and charged amino acids, Y is 1-3 hydrophobic amino acids, T is 2-8 turn sequence amino acids, and N and M are each independently between 2 and 10. Y amino acids may independently be selected from glycine, alanine, valine, leucine, isoleucine, proline, phenylalanine, methionine, threonine, and tryptophan. In some embodiments, the folding group sequence may be Y[AY]_{N}[T][YA]_{M}Y-NH₂.

Certain exemplary self-assembling peptides may have a folding group sequence comprising [XY]_{N}[T][YX]_{M}, where X is 1-3 charged amino acids, Y is 1-3 hydrophobic amino acids, T is 2-8 turn sequence amino acids, and N and M are each independently between 2 and 10. X amino acids may independently be selected from arginine, lysine, tryptophan, and histidine. Y amino acids may independently be selected from glycine, alanine, valine, leucine, isoleucine, proline, phenylalanine, methionine, threonine, and tryptophan. In some embodiments, the folding group sequence may be Y[XY]_{N}[T][YX]_{M}Y-NH₂.

Certain exemplary self-assembling peptides may have a folding group sequence comprising [ZY]_{N}[T][YZ]_{M}, where Z is 1-3 polar or charged amino acids, Y is 1-3 hydrophobic amino acids, T is 2-8 turn sequence amino acids, and N and M are each independently between 2 and 10. Z amino acids may independently be selected from glutamine, asparagine, histidine, serine, threonine, tyrosine, cysteine, alanine, valine, leucine, isoleucine, proline, phenylalanine, arginine, lysine, aspartic acid, and glutamic acid. Y amino acids may independently be selected from glycine, alanine, valine, leucine, isoleucine, proline, phenylalanine, methionine, threonine, and tryptophan. In some embodiments, the folding group sequence may be Y[ZY]_{N}[T][YZ]_{M}Y-NH₂.

One exemplary self-assembling peptide may have a folding group comprising -RYRYRYTYRYRYR- where R is an arginine residue, Y is 1-3 hydrophobic amino acids, and T is 2-6 turn sequence amino acids.

One exemplary self-assembling peptide may have a folding group comprising -VXVXVXVXVTXVXVXVXV- where V is a valine residue, X may be independently selected from charged and neutral amino acid residues serine, glutamic acid, lysine, tryptophan, and histidine, and T is 2-8 turn sequence amino acids. In some embodiments, the exemplary folding group may comprise a series of hydrophobic valine amino acid residues alternating with independently selected hydrophilic and/or neutral amino acid residues.

One exemplary self-assembling peptide may have a folding group comprising -KYKYKYTYKYKYK- where R is an arginine residue, Y is 1-3 hydrophobic amino acids, and T is 2-6 turn sequence amino acids.

One exemplary self-assembling peptide may have a folding group comprising -VZVZVZVTVZVZVZV- where V is a valine residue, Z is 1-3 hydrophilic amino acids, and T is 2-6 turn sequence amino acids.

Exemplary self-assembling peptides may have a turn sequence comprising 2-8 turn sequence amino acids, for example 2-5 turn sequence amino acids. The turn sequence amino acids may be selected from proline, for example D-proline and/or L-proline, aspartic acid, and asparagine. In some embodiments, the turn sequence may be (d)PP, (d)PG, or NG.

Exemplary self-assembling peptides having a turn sequence include VKVRVRVRV(d)PPTRVRVRVKV-NH₂ and VLTKVKTKV(d)PPTKVEVKVLV-NH₂. In the exemplary peptides, the tetrapeptide turn sequence (-V(d)PPT-) was selected to adopt a type II′ turn and positioned within the middle of the peptide sequence. This four-residue turn sequence occupies the i, i+1, i+2 and i+3 positions of the turn. The heterochiral sequence ((d)P (i+1)−P (i+2)) dipeptide was selected for its tendency to adopt dihedral angles consistent with type II′ turns. Incorporation of a bulky β-branched residue (valine) at the i position of the turn sequence enforces the formation of a trans prolyl amide bond at the i+1 position. The placement of valine at this position is selected to design against the formation of a cis prolyl bond, which results in β-strands that adopt an extended conformation rather than the intended hairpin. Threonine exhibits a statistical propensity to reside at the i+3 position. Therefore, threonine was selected to be incorporated at this position within the tetrapeptide sequence, which bears a side-chain hydroxyl group capable of hydrogen bonding to the amide backbone carbonyl at the i position, to further stabilize the turn.

The exemplary folding peptides may be designed to include high propensity β-sheet forming residues flanking the type II′ turn sequence. The selection of alternation of hydrophobic and hydrophilic residues along the strands provides an amphiphilic β-sheet when the peptide folds. For example, lysine may be chosen as a hydrophilic residue to provide a side chain pKa value of about 10.5. Side chain amines are generally protonated when dissolved under slightly acid conditions, forming unfavorable electrostatic interactions between β-strands of the hairpin and inhibiting peptide folding and self-assembly. However, as pH is increased to about pH 9, a sufficient portion of the lysine side chains become deprotonated allowing the peptide to fold into an amphiphilic β-hairpin. The electrostatic interactions may be employed to design pH responsiveness of the disclosed peptides.

While not wishing to be bound by theory, it is believed the amphiphilic β-hairpin is stabilized in the intramolecular folded state by van der Waals contacts between neighboring amino acid side chains within the same hairpin. The formation of intramolecular hydrogen bonds between cross β-strands of the hairpin and the propensity for the turn sequence to adopt at type II′ turn may further stabilize the folded conformation. Once in the folded state, the lateral and facial associations of the β-hairpins may be selected to design self-assembly. For example, lateral association of β-hairpins promotes the formation of intermolecular hydrogen bonds and van der Waals contacts between neighboring amino acids.

Exemplary self-assembling peptides may have a folding group sequence comprising (X)a(Y)b(Z)c-[(d)PP, (d)PG, or NG]-(Z)c(Y)b(X)a, where the turn sequence is (d)PP, (d)PG, or NG, (d)P is a D-proline, X is a charged amino acid, Y is a hydrophobic amino acid, Z is a hydrophobic amino acid or a polar amino acid, and a, b, and c are each independently an integer from 1-10. In certain embodiments, X is independently selected from valine, leucine, isoleucine, phenylalanine, tryptophan, tyrosine, threonine, and combinations thereof. In certain embodiments, Y is independently selected from glutamic acid, serine, alanine, proline, aspartic acid, and combinations thereof. In some embodiments, Z is independently selected from glutamine, glutamic acid, lysine, arginine, and combinations thereof.

Exemplary self-assembling peptides may have a folding group sequence comprising (Z)c(Y)b(X)a-[(d)PP, (d)PG, or NG]-(X)a(Y)b(Z)c, where the turn sequence is (d)PP, (d)PG, or NG, (d)P is a D-proline, X is a charged amino acid, Y is a hydrophobic amino acid, Z is a hydrophobic amino acid or a polar amino acid, and a, b, and c are each independently an integer from 1-10. In certain embodiments, X is independently selected from valine, leucine, isoleucine, phenylalanine, tryptophan, tyrosine, threonine, and combinations thereof. In certain embodiments, Y is independently selected from glutamic acid, serine, alanine, proline, aspartic acid, and combinations thereof. In some embodiments, Z is independently selected from glutamine, glutamic acid, lysine, arginine, and combinations thereof.

Any of the charged, hydrophobic, polar, or amphipathic amino acids disclosed herein may derive one or more of their properties from the composition of the biocompatible solution.

Hydrophobic amino acids are those which tend to repel water. Hydrophobic amino acids include alanine, valine, leucine, isoleucine, proline, tyrosine, tryptophan, phenylalanine, methionine, and cysteine. The hydrophobic amino acids may be independently selected from alanine, valine, leucine, isoleucine, proline, phenylalanine, methionine, cysteine, and combinations thereof. In some embodiments, the hydrophobic amino acids comprise valine.

Charged amino acids are those which tend to have an electric charge under the given conditions. Charged amino acids may have side chains which form salt bridges. Charged amino acids include alanine, valine, leucine, isoleucine, proline, phenylalanine, cysteine, arginine, lysine, histidine, aspartic acid, and glutamic acid. The folding group may comprise 2-10 charged amino acids, for example 2-8 charged amino acids.

The charged amino acids may be positively charged amino acids. The folding group may comprise 2-10 charged amino acids, for example 2-8 charged amino acids. The positively charged amino acids may be independently selected from arginine, lysine, histidine, and combinations thereof. The folding group may comprise 2-8 arginine residues, lysine residues, or a combination of arginine and lysine residues. In some embodiments, the folding group may comprise 6 positively charged residues selected from arginine, lysine, or a combination of arginine and lysine.

The charged amino acids may be negatively charged amino acids. The folding group may comprise 2-10 negatively charged amino acids, for example, 2-8 negatively charged amino acids. In some embodiments, the negatively charged amino acids may be independently selected from aspartic acid, glutamic acid, and combinations thereof.

Polar amino acids are those which have an uneven charge distribution. Polar amino acids may tend to form hydrogen bonds as proton donors or acceptors. Polar amino acids include glutamine, asparagine, histidine, serine, threonine, tyrosine, and cysteine.

Amphipathic amino acids are those which have both a polar and non-polar component. Amphipathic amino acids may be found at the surface of proteins or lipid membranes. Amphipathic amino acids include tryptophan, tyrosine, and methionine.

Exemplary self-assembling peptides may have a folding group sequence of any of SEQ ID NOS: 1-23. The self-assembling peptide may have a folding group sequence of SEQ ID NO: 1. In certain embodiments, the self-assembling peptide may have a folding group sequence of SEQ ID NO: 2. The self-assembling peptide may have a folding group sequence of SEQ ID NO: 3. The self-assembling peptide may have a folding group sequence of SEQ ID NO: 4. The self-assembling peptide may have a folding group sequence of SEQ ID NO: 5. The self-assembling peptide may have a folding group sequence of SEQ ID NO: 6. The self-assembling peptide may have a folding group sequence of SEQ ID NO: 7. The self-assembling peptide may have a folding group sequence of SEQ ID NO: 8. The self-assembling peptide may have a folding group sequence of SEQ ID NO: 9. The self-assembling peptide may have a folding group sequence of SEQ ID NO: 10. The self-assembling peptide may have a folding group sequence of SEQ ID NO: 11. The self-assembling peptide may have a folding group sequence of SEQ ID NO: 12. The self-assembling peptide may have a folding group sequence of SEQ ID NO: 13. The self-assembling peptide may have a folding group sequence of SEQ ID NO: 14. The self-assembling peptide may have a folding group sequence of SEQ ID NO: 15. The self-assembling peptide may have a folding group sequence of SEQ ID NO: 16. The self-assembling peptide may have a folding group sequence of SEQ ID NO: 17. The self-assembling peptide may have a folding group sequence of SEQ ID NO: 18. The self-assembling peptide may have a folding group sequence of SEQ ID NO: 19. The self-assembling peptide may have a folding group sequence of SEQ ID NO: 20. The self-assembling peptide may have a folding group sequence of SEQ ID NO: 21. The self-assembling peptide may have a folding group sequence of SEQ ID NO: 22. The self-assembling peptide may have a folding group sequence of SEQ ID NO: 23.

Exemplary self-assembling peptides which have shear-thinning properties include VKVRVRVRV(d)PPTRVRVRVKV-NH₂, and VKVRVRVRV(d)PPTRVEVRVKV-NH₂ (which has a single substitution of glutamic acid at position 15 on the hydrophilic face). The glutamic acid substitution results in a faster rate of gelation of the self-assembling peptide gel in the presence of ionic salts in the biocompatible solution. The negatively charged glutamic acid lowers the overall positive charge of the peptide and enables faster folding and self-assembly.

Exemplary self-assembling peptides which have shear-thinning properties that can be tuned for net peptide charge include VKVRVRVRV(d)PPTRVEVRVKV-NH₂, and VKVKVKVKV(d)PPTKVEVKVKV-NH₂, (which has an arginine substituted for lysine on the hydrophilic face). The lysine substitution lowers the peptide net charge at physiological pH that allows for better mammalian cell cytocompatibility when compared to peptides with high arginine content (higher net charge). The exemplary peptides are antimicrobial self-assembling peptides.

Exemplary self-assembling peptides which have shear-thinning properties that can be tuned for peptide gels with faster rate of gelation and increased stiffness include FKFRFRFRV-(d)PPTRFRFRFKF-NH₂, (which has valine substituted for phenylalanine on the hydrophobic face). The phenylalanine substitution increases the hydrophobic face of the peptide that allows for stiffer and faster gelation of peptide gels. The exemplary peptides are antimicrobial self-assembling peptides.

Exemplary self-assembling peptides which have shear-thinning properties include enantiomer forms of the exemplary sequences listed above, such as an enantiomer form of VKVRVRVRV(d)PPTRVRVRVKV-NH₂, (d)V(d)K(d)V(d)R(d)V(d)R(d)V(d)R(d)V(L)P(d)P (d)T(d)R(d)V(d)R(d)V(d)R(d)V(d)K(d)V-NH₂, (which has D isoforms of the sequence and an L isoform of P). The isoform substitution may provide control of peptide degradation and increased stability without compromising peptide net charge at physiological pH. The sequence may provide better compatibility with mammalian cells. The peptide may be a complete enantiomer (as shown above) or a partial enantiomer such that any one or more of the amino acids may be an enantiomer of the sequences listed above. The exemplary peptides are antimicrobial self-assembling peptides.

Other exemplary self-assembling peptides include Ac-VEVSVSVEV(d)PPTEVSVEVEV GGGGRGDV-NH₂ and VEVSVSVEVdPPTEVSVEVEV-NH₂.

The self-assembling peptide may comprise at least one guanidine moiety. The guanidine moiety may be associated with an organic molecule which makes up part of the peptide chain. In exemplary embodiments, a guanidine group may be incorporated as part of the side chain of an arginine residue. However, the peptide may comprise guanidine moieties which are not associated with arginine residues.

A guanidine moiety is generally a highly polar group which, when positioned on a cationic peptide, may allow for pairing with hydrophobic and hydrophilic groups forming salt bridges and hydrogen bonds. Such a peptide may display a high capacity to penetrate cell membranes and provide antimicrobial activity. The guanidine moiety may also promote peptide stability by improving peptide folding, physical characteristics and thermal stability of the peptide and/or hydrogel.

The peptide may generally have 20-50% guanidium content, as measured by number of guanidine groups by total number of amino acid residues of the peptide. For instance, an exemplary peptide sequence having 20 amino acid residues, of which 6 are arginine residues having a guanidine group, has 30% guanidium content. The exemplary peptides may penetrate and disrupt cell membranes.

### Properties of the Peptide Hydrogel Preparation

The preparation may generally comprise the self-assembling peptide in a biocompatible solution. For example, the peptide may be dissolved or substantially dissolved in the biocompatible solution. The preparation may comprise between about 0.1% w/v and about 10.0% w/v of the peptide. The preparation may be formulated for a target indication. For instance, the concentration of the self-assembling peptide may be selected based on a target indication. For example, an exemplary preparation having antimicrobial properties may comprise less than 1.5% w/v of the peptide, for example, between about 0.5% w/v of the peptide and 1.0% w/v of the peptide.

Exemplary preparations may comprise between about 0.1% w/v and about 10.0% w/v of the peptide, for example, between about 0.15% w/v and about 10.0% w/v of the peptide, between about 0.1% w/v and about 8.0% w/v of the peptide, between about 0.25% w/v and about 6.0% w/v of the peptide, between about 0.5% w/v and about 6.0% w/v of the peptide. When the peptide is purified, the preparation may comprise up to about 6.0% w/v of the peptide. In certain embodiments, the preparation may comprise less than about 3.0% w/v of the peptide, for example, between about 0.25% w/v and about 3.0% w/v of the peptide, between about 0.25% w/v and about 2.0% w/v of the peptide, between about 0.25% w/v and about 1.25% w/v of the peptide, or between about 0.5% w/v of the peptide and about 1.5% w/v of the peptide. The preparation may comprise between about 0.5% w/v and about 1.0% w/v of the peptide, between about 0.7% w/v and about 2.0% w/v of the peptide, or between about 0.7% w/v and about 0.8% w/v of the peptide. For instance, the preparation may comprise about 0.25% w/v, about 0.5% w/v, about 0.7% w/v, about 0.75% w/v, about 0.8% w/v, about 1.0% w/v, about 1.5% w/v of the peptide, about 2.0% w/v, or about 3.0% w/v. In particular embodiments, the preparation may comprise less than about 1.5% w/v of the peptide. The preparation may comprise less than about 1.25% w/v of the peptide or less than about 1.0% w/v of the peptide. In one exemplary embodiment, the preparation may comprise about 0.75% w/v of the peptide.

After combination with the buffer, the hydrogel may have between about 0.05% w/v and 6.0% w/v of the peptide. For example, the hydrogel may have between about 0.1% w/v, and 6.0% w/v of the peptide, between about 0.25% w/v and 6.0% w/v of the peptide, between about 1.5% w/v and 6.0% w/v of the peptide, between about 0.25% w/v and 3.0% w/v of the peptide, between about 0.25% w/v and 1.0% w/v of the peptide, between about 0.25% w/v and 0.5% w/v of the peptide, or between about 0.3% w/v and 0.4% w/v of the peptide. The peptide preparation and buffer may be combined to form the hydrogel at a ratio of between about 2:1 to 0.5:1 peptide preparation to buffer. In some embodiments, the peptide preparation and buffer may be combined to form the hydrogel at a ratio of about 1:1.

The peptides in the preparation may be purified. As disclosed herein, “purified” may refer to compositions treated for removal of contaminants. In particular, the purified peptides may have a composition suitable for clinical application. For example, the peptides may be purified to meet health and/or regulatory standards for clinical administration. The peptide may be at least 80% purified, for example, at least 85%, at least 90%, at least 92%, at least 95%, at least 98%, at least 99%, or at least 99.9%.

In certain embodiments, the peptides may be purified to remove or reduce residual organic solvent content from solid phase synthesis of the peptides. The peptide may comprise less than 20% residual organic solvent by weight. The peptide may comprise less than 15% residual organic solvent by weight. The peptide may comprise less than 10% residual organic solvent by weight. For example, the peptide may comprise less than 8%, less than 5%, less than 2%, less than 1%, or less than 0.1% residual organic solvent by weight. Exemplary organic solvents which may be removed or reduced from the synthesized peptide include trifluoroacetic acid (TFA), acetonitrile, isopropanol, N,N-Dimethylformamide, triethylamine, Ethyl Ether, and acetic acid.

The purified peptides may be substantially free of Trifluoroacetic acid (TFA). For example, the purified peptides may have less than 1% w/v residual TFA, or between about 0.005% w/v and 1% w/v residual TFA.

The purified peptide may be substantially free of acetonitrile. In some embodiments, the purified peptide may have less than about 410 ppm residual acetonitrile. The purified peptide may have between about 0.005 ppm and about 410 ppm residual acetonitrile.

The purified peptide may be substantially free of isopropanol. In some embodiments, the purified peptide may have less than about 400 ppm residual isopropanol. The purified peptide may have less than about 100 ppm residual isopropanol. The purified peptide may have between about 0.005 ppm and 100 ppm residual isopropanol.

The purified peptide may be substantially free of N,N-Dimethylformamide. In some embodiments, the purified peptide may have less than about 880 ppm residual N,N-Dimethylformamide. The purified may have between about 0.005 ppm and about 880 ppm residual N,N-Dimethylformamide.

The purified peptide may be substantially free of triethylamine. In some embodiments, the purified peptide may have less than about 5000 ppm residual triethylamine. The purified peptide may have between about 0.005 ppm and about 5000 ppm residual triethylamine.

The purified peptide may be substantially free of Ethyl Ether. In some embodiments, the purified peptide may have less than about 1000 ppm residual Ethyl Ether. The purified peptide may have between about 0.005 ppm and about 1000 ppm residual Ethyl Ether.

The purified peptides may be substantially free of acetic acid. For example, the purified peptides may have less than 2% w/v residual acetic acid, for example, less than 1% w/v residual acetic acid, less than 0.5% w/v residual acetic acid, less than 0.1% w/v residual acetic acid, between about 0.0001% w/v and 2% w/v residual acetic acid, or between about 0.005% w/v and 0.1% w/v residual acetic acid.

In general, the purified peptide and/or biocompatible solution may have properties consistent with regulatory limits defined by the International Council for Harmonization of Technical Requirements for Pharmaceuticals for Human Use (ICH).

The biocompatible solution of the preparation may refer to a substantially liquid carrier for the peptide. The biocompatible solution may generally be an aqueous solution. The biocompatible solution may comprise water, for example, deionized water. Deionized water may have a resistivity of greater than about 18 MΩ and a conductivity of less than about 0.056 μS at 25° C. Deionized water may have a maximum endotoxin specification of 0.03 endotoxin units (EU)/ml and 1 CFU/mL microbial action or less. Deionized water may have a total organic carbon (TOC) concentration of 10 ppb or less. The biocompatible solution may comprise pharmaceutical grade water. Pharmaceutical grade water may have 500 ppb total organic carbon (TOC) or less and 100 CFU/ml microbial action or less. The biocompatible solution may comprise injection grade water. Injection grade water may have a maximum endotoxin specification of 0.25 endotoxin units (EU)/ml and 10 CFU/10 ml microbial action or less. In certain embodiments, the preparation or biocompatible solution may be substantially free of chloride ions.

The preparation or peptide may comprise counterions. As disclosed herein, a counterion may refer to a charge balancing ion. The preparation or peptide may have an effective amount of counterions to render the preparation substantially electrically neutral. The preparation or peptide may have an effective amount of counterions to render the preparation substantially biocompatible and/or stable. The preparation or peptide may have an effective amount of counterions to control repulsion of anionic or cationic residues of the peptide. The concentration of counterions may be dependent on the peptide sequence and concentration of the peptide and any additives. In exemplary embodiments, the peptide may comprise between 0.1-20% counterions. Additionally, the charge of the counterions may be dependent on the charge of the peptide and any additives. Thus, the counterions may be anions or cations. In general, the counterions may be cytocompatible. In certain embodiments, the counterions may be biocompatible. For instance, the counterions may comprise acetate, citrate, ammonium, fluoride, or chloride. In other embodiments, the preparation or peptide may be substantially free of chloride counterions.

In exemplary embodiments, the preparation or peptide may comprise an effective amount of acetate counterions. In particular, preparations having a peptide concentration which comprises residual TFA may have an amount of acetate counterions sufficient to balance the residual TFA. Briefly, TFA is commonly used to release synthesized peptides from solid-phase resins. TFA is also commonly used during reversed-phase HPLC purification of peptides. However, residual TFA or fluoride may be toxic and undesirable in peptides intended for clinical use. Furthermore, TFA may interact with the free amine group at the N-terminus and side chains of positively charged residues (for example, lysine, histidine, and arginine). The presence of TFA-salt counterions in the peptide preparation may be detrimental for biological material and may negatively affect the accuracy and reproducibility of the intended peptide activity.

TFA-acetate salt exchange by acetate or hydrochloride may be employed to counteract some or all of the negative effects of TFA described above. The inventors have determined the acetate counterion is surprisingly well suited for maintaining biological activity of the peptide preparation and for controlling solubility of the peptide and charge for self-assembly of the peptide. Furthermore, acetic acid (pKa=4.5) is weaker than both trifluoroacetic acid (pKa about 0) and hydrochloric acid (pKa=−7). Acetate counterions may additionally control pH of the peptide preparations to be physiologically neutral.

The preparation may have variable hydrogelation kinetics. In accordance with certain embodiments, the hydrogelation kinetics of the preparation may be designed for a particular mode of administration. The preparation may be administered as a liquid. The preparation may be administered as a solid or semi-solid. The preparation may be administered as a gel. The preparation may be administered as a combination of hydrogel suspended in a liquid. The preparation may have a variable apparent viscosity. For instance, the preparation may have an apparent viscosity effective to allow injection under the conditions of administration. In certain embodiments, the preparation may have an apparent viscosity which decreases with increasing shear stress.

The preparation may be configured to reversibly self-assemble and disassemble in response to applied stress, for example, applied mechanical force. The solid or gel preparation may become disrupted with increasing applied stress, to be later restored once the applied stress is reduced. The solid or gel may become fluid in response to applied stress, for example, during delivery through a delivery device. The peptide may be capable of undergoing sequential phase transitions in response to applied stress. The peptide may be capable of recovering after each one or more sequential phase transitions.

The preparation may be configured to reversibly self-assemble and disassemble responsive to at least one of change in temperature, change in pH, contact with an ion chelator, dilution with a solvent, applied sound wave, lyophilization, vacuum drying, and air drying. The administered fluid may conform to tissue voids before reforming as a solid or gel. Thus, the solid or gel preparation may be injectable, flowable, or sprayable under the appropriate shear stress. Once administered, the preparation may be restored to a solid or gel form, substantially conforming to the target site. The formation may occur within less than a minute, about one minute, less than about 2 minutes, less than about 3 minutes, less than about 5 minutes, or less than about 10 minutes. The formation may occur within about one minute, less than about 30 seconds, less about 10 seconds, or about 3 to 5 seconds.

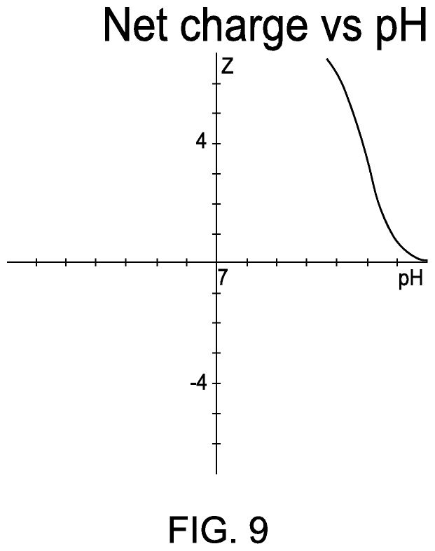

The peptide may be purified. For example, the peptide may be lyophilized. As shown in FIG. 9, net charge may be quantified as a function of pH value. The exemplary peptide measured in FIG. 9 is an arginine-rich peptide having two lysine residues. The exemplary peptide of FIG. 9 has a net charge of +9 at a pH of 7. Other peptides are within the scope of the disclosure. For example, the purified peptide may have a net charge between-9 to +11 at pH 7, for example, −7 to +9 at pH 7. As disclosed herein, “net charge” may refer to a total electric charge of the peptide as a biophysical and biochemical property, typically as measured at a pH of 7.

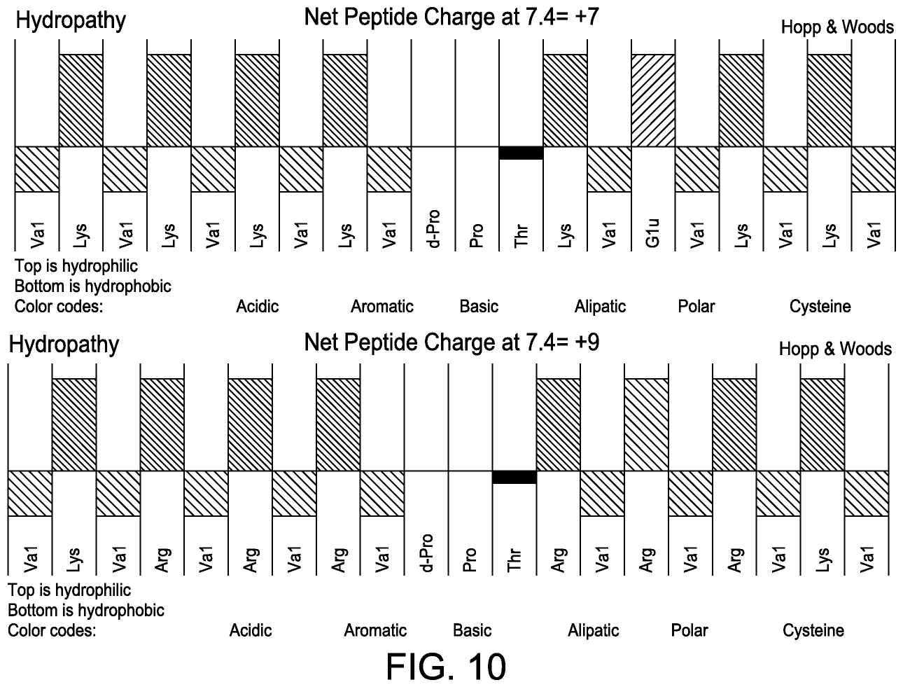

The purified peptide may have a net charge of from −7 to +11 at pH 7. In some embodiments, the peptide may have a net charge of from +2 to +9, for example, +5 to +9 or +7 to +9. The purified peptide may have a charge of about +5, +6, +7, +8, +9, +10, or +11 at pH 7. Exemplary peptides having a charge of +5 to +9 include VLTKVKTKV(d)PPTKVEVKVLV, VKVRVRVRV(d)PPTRVRVRVKV, and VKVRVRVRV(d)PPTRVEVRVKV. In other embodiments, the purified peptide may be substantially neutral. In other embodiments, the peptide may have a net negative charge. An exemplary peptide having a net negative charge is VEVSVSVEV(d)PPTEVSVEVEV. As shown in FIG. 10, a single substitution of glutamic acid in the peptide sequence may alter net peptide charge from +7 (top panel) to +9 (bottom panel) at pH 7, as well as alter isoelectric point from 11.45 to 14. Net charge may be selected by peptide design. Design of electrostatic charge in the peptide hydrogel may allow control of charge interaction with cell membrane and proteins.

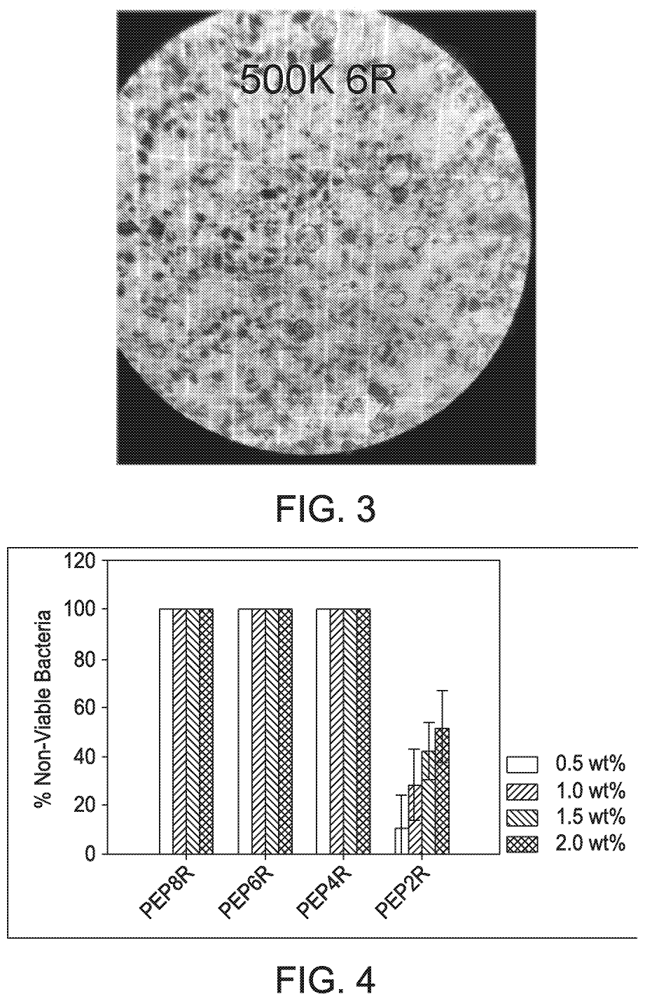

The peptide may be designed to have a charge that adsorbs and/or promotes deactivation of proteins at a target site of administration. For instance, positively charged peptide hydrogels may promote adsorption of negatively and neutrally charged molecules such as small molecules, proteins, and extravesicular membranes. Negatively charged peptide hydrogels may promote adsorption of positively and neutrally charged molecules such as small molecules, proteins, and extravesicular membranes. Furthermore, the peptide may be designed to have regions of positive, neutral, or negative charge, to varying degrees. In certain embodiments, the peptide charge may be designed such that when placed into a rich solution of charged molecules, the peptide may soak out or absorb the molecules into the hydrogels attaching the molecules to the peptides by adsorption. FIG. 3 is a microscopy image showing negatively charged Trypan blue adsorbed on a positively charged hydrogel.

The purified peptide may have greater than 70% w/v, greater than 80% w/v, or greater than 90% w/v nitrogen, for example, between 70% w/v and 99.9% w/v nitrogen.

The purified peptide may have a bacterial endotoxin level of less than about 10 EU/mg, for example, less than about 5 EU/mg, less than about 2 EU/mg, or less than about 1 EU/mg. In other embodiments, the purified peptide may have an endotoxin level of between about −0.010 to −0.015 EU/ml. For instance, the purified peptide may have an OD at 410 nm of between 0.004 to 0.008, for example, about 0.006. The peptide hydrogel may have an OD at 410 nm of between 0.010 to 0.020, for example, about 0.015. In some embodiments, the purified peptide and/or preparation may be substantially free of endotoxins.

The purified peptide in the biocompatible solution may have a water content of between about 1% w/v and about 20% w/v, for example, at least about 10% w/v, or less than about 15% w/v.

The purified peptide may have an isoelectric point of between about 7-14. For example, the purified peptide may have an isoelectric point of about 7, 8, 9, 10, 11, 12, 13, or 14.

The purified peptide may be configured to self-assemble into a hydrogel having a shear modulus of between about 2 Pa to 3500 Pa as determined by rheology testing. For example, the purified peptide may self-assemble into a hydrogel having a shear modulus of greater than 100 Pa, between 100 Pa and 3500 Pa, between 100 Pa and 3000 Pa, between 2 Pa and 1000 Pa, or between 2 Pa and 500 Pa. For example, a formulation having 0.75% w/v peptide may have a shear modulus of between about 2 Pa and 500 Pa. A formulation having 1.5% w/v peptide may have a shear modulus of between about 100 Pa and 3000 Pa. A formulation having 3.0% w/v peptide may have a shear modulus of between about 1000 Pa and 10000 Pa. Thus, shear modulus of the hydrogel may be controlled by selection of peptide concentration in the formulation.

The peptide may be designed to adopt a predetermined secondary structure. For example, the peptide may be designed to adopt a β-hairpin secondary structure, as previously described. The predetermined secondary structure may comprise a structure preselected from at least one of a β-sheet, an α-helix, and a random coil. In exemplary embodiments, the hydrophobic amino acid residues (for example, quantity, placement, and/or structure of the hydrophobic amino acid residues) may be selected to self-assemble the peptide into a polymer having a majority of β-sheet structures. In particular embodiments, the hydrophobic amino acid residues may be selected to control stiffness of the hydrogel. For example, an amount and type of hydrophobic amino acid residues may be selected to control stiffness of the hydrogel.

In some embodiments, an external stimulus such as temperature, change in pH, light, and applied sound wave may be used to control and promote preferential secondary structure formation of the self-assembling peptide. Control of the secondary structure formation may enhance biological, biophysical, and chemical therapeutic functions of the peptide. For example, higher cell membrane penetration of self-assembling peptides may be achieved by exposing β-hairpin peptides to high pH (for example, at least pH 9) or high temperatures (for example, at least 125° C.) or low temperatures (for example, 4° C. or lower). The result is hydrogels with a peptide secondary structure having a majority β-sheet or α-helix formation.

The peptide may be designed to give the preparation shear-thinning properties. In particular, the peptide may be designed to be injectable. For instance, the peptide may be designed to be an injectable solid or gel by employing shear-thinning kinetics. The preparation, in the form of a solid or gel prior to application, may be configured to shear-thin to a flowable state under an effective shear stress applied during administration by the delivery device. In some exemplary embodiments, the solid or gel may shear-thin to a flowable state during injection or topical application with a syringe. Other modes of administration may be employed. The solid or gel may shear-thin to a flowable state during endoscopic administration. The solid or gel may be configured to shear-thin to flow through an anatomical lumen, for example, an artery, vein, gastrointestinal tract, bronchus, renal tube, genital tract, etc. In some embodiments, the shear thinning properties may be employed during transluminal procedures. The peptide may be designed to be sprayable. For example, the peptide may be designed for administration as a spray or other liquid droplet, for example, other propelled liquid droplet, by employing shear-thinning kinetics, as previously described.

The shear-thinning kinetics of the hydrogel may be engineered by altering the net charge of the peptides. In some embodiments, the net charge may be altered by controlling one or more of the presence or absence of cationic particles or peptides, the presence or absence of anionic particles or peptides, buffers, salts, peptide concentration, peptide purity, and the presence or absence of peptide counterions. In particular, shear-thinning may be controlled by altering the peptide purity to achieve the desired shear-thinning kinetics. The net charge of the peptide may be positive. The net charge of the peptide may be negative.

Shear-thinning may be induced by mechanical agitation to the hydrogel or environmental stimulus. Mechanical agitation may be induced, for example, through delivery or sonication mixing. Environmental stimulus may be induced by addition of heat, light, ionic agents, chelator agents, buffers, or proteins, or altering pH level.

Thus, the preparations may be substantially flowable. The methods may include dispensing the preparation through a cannula or needle. The methods may include conformally filling wound beds of any size and shape. The peptide hydrogels may have shear-thinning mechanical properties. The shear-thinning mechanical properties may allow the gel network to be disrupted and become a fluid during administration, for example, during injection from a needle or administration with a spray nozzle. When the applied stress ceases, the gel network may reform and the elastic modulus may be restored within a predetermined period of time, for example, several minutes. The shear-thinning peptide hydrogels may be employed to protect cells from damage during injection, showing an improved viability over direct injection in saline or media. The shear-thinning hydrogel may display non-Newtonian fluid flow, which may allow for effective mixing of excipients, for example, within minutes to a couple hours. In some embodiments, dyes, small molecules, and large molecules may be substantially homogeneously dispersed within the hydrogel in less than 120 minutes, for example, between 30-120 minutes.

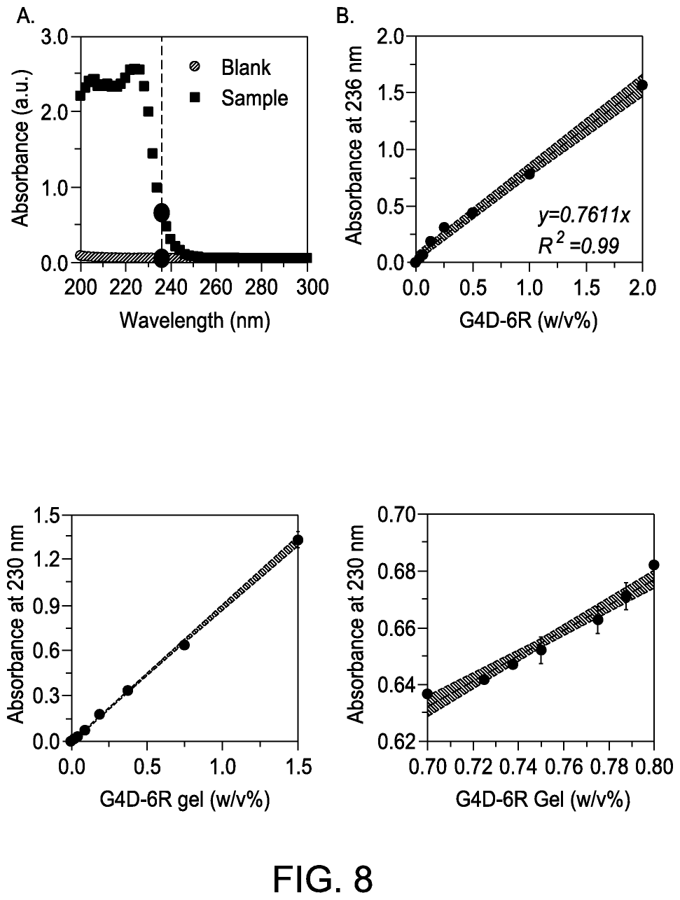

The peptide may self-assemble into a translucent hydrogel. In some embodiments, the peptide may self-assemble into a substantially transparent hydrogel. The transparency of the hydrogel may enable a user or healthcare provider to view surrounding tissues through the hydrogel. In exemplary embodiments, a surgical procedure may be performed without substantial obstruction of view by the hydrogel. The hydrogel may have at least about 70%, at least about 80%, at least about 90%, at least about 95%, at least about 99%, or about 100% light transmittance. The hydrogel may be colorless. The light transmittance and color of the hydrogel may be engineered by tuning the sequence of the peptide and/or the composition of the preparation or solution. As shown in the graphs of FIG. 8, transparency of the peptide hydrogels may be quantified by absorbance measurements. The exemplary peptide hydrogels measured in FIG. 8 are substantially transparent.

In some embodiments, the preparation may include a dye. The dye may be a food-grade dye or a pharmaceutical-grade dye. The dye may be cytocompatible. The dye may be biocompatible. In general, the dye may assist the user or healthcare provider to view the hydrogel after application. The preparation may include an effective amount of the dye to provide a desired opacity of the hydrogel. The hydrogel may comprise an effective amount of the dye to have a light transmittance of less than about 60%, less than about 50%, less than about 40%, less than about 30%, less than about 20%, or less than about 10%. The hydrogel may be substantially opaque when including the dye.

The peptide may self-assemble into a substantially ionically-crosslinked hydrogel. “Ionic crosslinkage” may refer to ionic bonds between peptides to form secondary structure proteins and/or between secondary structure proteins that form the hydrogel tertiary structure. The shear-thinning properties of the hydrogel may be enabled by physical crosslinks, allowing ionic bonds to be broken and reformed. In accordance with certain embodiments, the hydrogel is formed of a majority of ionic crosslinks. For example, more than 50%, more than 60%, more than 70%, more than 80%, more than 90%, more than 95%, more than 99%, or substantially all of the physical crosslinks of the formed hydrogel may be ionic in nature.

The preparation and/or assembled hydrogel may be designed to have a substantially physiological pH level. The preparation or hydrogel may have a pH level of between about 4.0 and 9.0. In some embodiments, the preparation or hydrogel may have a pH level of between about 7.0 and 8.0. The preparation or hydrogel may have a pH level of between about 7.3 and 7.5. The substantially physiological pH may allow administration of the preparation at the time of gelation. In some embodiments, the hydrogel may be prepared at a point of care. The methods may comprise mixing the preparation with a buffer configured to induce self-assembly, optionally agitating the mixture, and administering the preparation or hydrogel at a point of care. The administration may be topical or parenteral, as described in more detail below.

The peptide may be designed to self-assemble in response to a stimulus. The stimulus may be an environmental stimulus, e.g., change in temperature (e.g., application of heat), exposure to light, change in pH, applied sound waves, or exposure to ionic agents, chelator agents, or proteins. The stimulus may be mechanical agitation, e.g., induced through delivery, sonication, or mixing. In some embodiments, the methods may comprise administering the preparation as a liquid. The methods may comprise administering the preparation as a gel. The methods may comprise administering the preparation as a solid or semi-solid.

In some embodiments, the preparation may be designed to self-assemble after a lapsed period of time. For example, the preparation may be designed such that the peptide is configured to begin self-assembly in less than about 5 minutes, in less than about 3 minutes, in less than about 2 minutes, in less than about 30 seconds, in less than about 10 seconds, or in less than about 3 seconds. In certain embodiments, the preparation may be designed such that the peptide is configured to self-assemble, i.e. be substantially self-assembled, within about 60 minutes, within about 30 minutes, within about 15 minutes, within about 10 minutes, within about 5 minutes, within about 3 minutes, within about 2 minutes, within about 30 seconds, within about 10 seconds, within about 5 seconds, or within about 3 seconds. The preparation may have a composition configured to control timing of self-assembly. For example, the preparation may have a composition configured for timed release of ionic agents or pH-altering agents. In certain embodiments, the sequence or structure of the peptide may be designed to control self-assembly of the peptide.

In some embodiments, the methods may comprise combining the preparation with a buffer. The “buffer” may refer to an agent configured to induce gelation, prior to, subsequently to, or concurrently with administration of the preparation to the subject. Thus, in some embodiments, the preparation may comprise a buffer. For example, the preparation may comprise or be combined with up to about 1000 mM of the buffer. The buffer may comprise an effective amount of ionic salts and a buffering agent, for example, to induce gelation and/or provide desired properties. For example, the buffer may be formulated to control or maintain pH of the preparation.

In particular embodiments, the buffer may have an effective amount of ionic salts to control stiffness of the hydrogel. The “ionic salt” may refer to a compound which dissociates into ions in solution. The buffer may comprise between about 5 mM and 400 mM ionic salts. For example, the buffer may comprise between about 5 mM and 200 mM ionic salts, between about 50 mM and 400 mM ionic salts, between about 50 mM and 200 mM ionic salts, or between about 50 mM and 100 mM ionic salts. The ionic salt may be one that dissociates into at least one of sodium, potassium, calcium, magnesium, iron, ammonium, pyridium, quaternary ammonium, chloride, citrate, acetate, and sulfate ions. The ionic salts may comprise sodium chloride, ammonium chloride, magnesium chloride, potassium chloride, calcium chloride, ammonium sulfate, magnesium sulfate, sodium sulfate, potassium sulfate, calcium sulfate, sodium bicarbonate, and combinations thereof.

In exemplary embodiments, the buffer may comprise between about 1 mM and about 200 mM sodium chloride. For example, the buffer may comprise between about 10 mM and about 150 mM sodium chloride, for example between about 50 mM and about 100 mM sodium chloride.

The buffer may comprise counterions. The buffer may have an effective amount of counterions to render the hydrogel substantially electrically neutral. The buffer may have an effective amount of counterions to induce self-assembly of the peptide. The concentration of counterions may be dependent on the composition of the peptide preparation. Additionally, the charge of the counterions may be dependent on the charge of the peptide preparation. Thus, the counterions may be anions or cations. In general, the counterions may be cytocompatible. In certain embodiments, the counterions may be biocompatible. For instance, the counterions may comprise acetate or chloride. In other embodiments, the biocompatible solution may be substantially free of chloride counterions.

The buffer may comprise from about 1 mM to about 150 mM of a biological buffering agent. For example, the buffer may comprise from about 1 mM to about 100 mM of a biological buffering agent, from about 1 mM to about 40 mM of a biological buffering agent, or from about 10 mM to about 20 mM of a biological buffering agent. The biological buffering agent may be selected from Bis-tris propane (BTP), 4-(2-hydroxyethyl)-1-piperazineethanesulfonic acid (HEPES), Dulbecco's Modified Eagle Medium (DMEM), tris(hydroxymethyl)aminomethane (TRIS), 2-(N-Morpholino) ethanesulfonic acid hemisodium salt, 4-Morpholineethanesulfonic acid hemisodium salt (MES), 3-(N morpholino) propanesulfonic acid (MOPS), and 3-(N-morpholino) propanesulfonic acid (MOBS), Tricine, Bicine, (tris(hydroxymethyl)methylamino) propanesulfonic acid (TAPS), N-(2-Acetamido)-2-aminoethanesulfonic acid (ACES), β-Hydroxy-4-morpholinepropanesulfonic acid, 3-Morpholino-2-hydroxypropanesulfonic acid (MOPSO), (N,N-bis(2-hydroxyethyl)-2-aminoethanesulfonic acid) (BES) and combinations thereof. Other biological buffering agents are within the scope of the disclosure.

In exemplary embodiments, the buffer may comprise from about 1 mM to about 150 mM of BTP. The buffer may comprise from about 10 mM to about 100 mM BTP, for example, from about 10 mM to about 50 mM BTP, from about 10 mM to about 40 mM, from about 20 mM to about 40 mM, or from about 20 mM to about 40 mM.

The buffer may additionally comprise at least one of water, an acid, and a base. The acid and/or base may be added in an amount effective to control pH of the buffer to be a substantially physiological pH. In other embodiments, the buffer may be acidic, alkali, or substantially neutral. The buffer may be selected to control pH of the hydrogel and maintain a desired pH at the target site. For example, to control pH of the hydrogel to be a substantially physiological pH at the target site. Thus, the properties of the buffer may be selected based on the target site. The buffer may have additional properties as selected, for example, net charge, presence or absence of additional proteins, etc. The buffer may additionally comprise one or more minerals.

The preparation may further comprise an effective amount of a mineral clay. The preparation may comprise between about 0.1% w/v to about 20% w/v of the mineral clay. For example, the preparation may comprise 0.75% w/v, 1.5% w/v, 2% w/v, 3% w/v, 4% w/v, 8% w/v, 10% w/v, or 20% w/v of the mineral clay. The amount of the mineral clay may be effective to provide desired rheological properties for the target site of application. The amount of the mineral clay may be effective to form a film. The mineral clay may be natural or synthetic. The mineral clay may comprise at least one of laponite and montmorillonite. In some embodiments, the preparation may comprise from a 1:1 to 1:2 ratio (w/v) of the peptide to mineral clay. For example, the ratio of peptide to mineral clay in the preparation may be 1:1, 3:4, 3:8, or 1:2 (w/v).

The preparation may be formulated for a target indication. For instance, the preparation may be formulated for treatment of a microbial infection or inhibition of proliferation of a microorganism, such as a pathogenic microorganism. The preparation may be formulated for treatment of a fungal infection or inhibition of proliferation of a fungal organism. The preparation may be formulated for cell culture and/or cell delivery. The preparation may be formulated for treatment or inhibition of a wound, such as a chronic wound, or biofilm. The preparation may be formulated by engineering the peptide as described in more detail below. The preparation may be formulated by selecting the biocompatible solution and/or additives.

In certain embodiments, the preparation may be formulated for a combination treatment. The preparation may include at least one active agent configured to provide a combination treatment. In some embodiments, the preparation may exhibit synergistic results with combination of the active agent. The active agent may be, for example, an antibacterial composition, an antiviral composition, an antifungal composition, an anti-tumor composition, an anti-inflammatory composition, a hemostat, a cell culture media, a cell culture serum, an anti-odor composition, an analgesic, local anesthetic, or a pain-relief composition. The preparation may be formulated for administration in conjunction with one of the aforementioned compositions. The preparation may be formulated for simultaneous or concurrent combination administration. The preparation may be formulated for sequential combination administration.

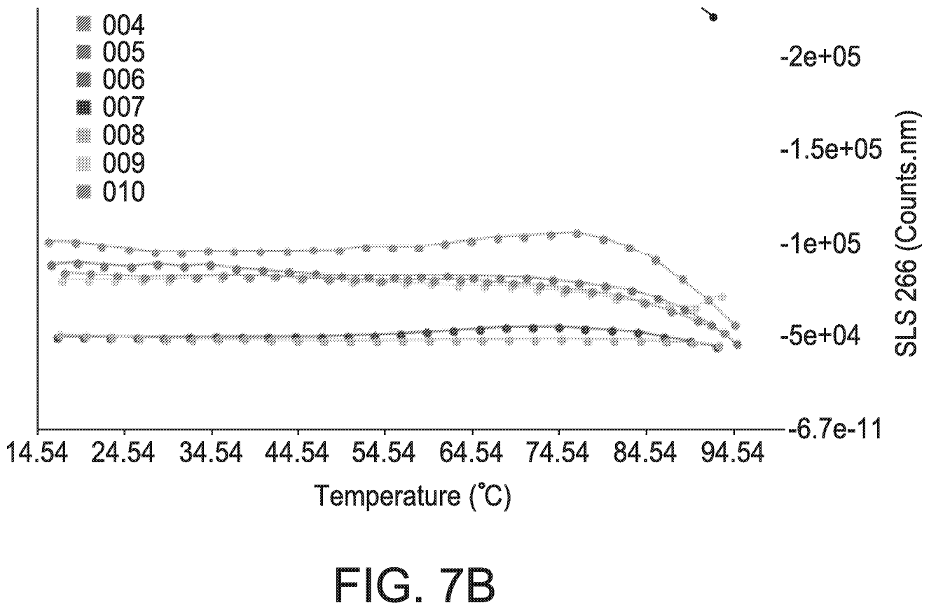

In some embodiments, the preparation and/or hydrogel may be designed to be thermally stable between −20° C. and 150° C., between −20° C. and 125° C., between −20° C. and 100° C., between 2° C. and 125° C., and between 37° C. and 125° C. As disclosed herein, “thermal stability” refers to the ability to withstand temperature treatment without substantial degradation, loss of biological activity, or loss of chemical activity. The graphs of FIGS. 7A-7B show peptide aggregation as measured by static light scattering (SLS) at 266 nm of exemplary peptides as a function of temperature. The exemplary peptides include arginine, lysine, valine, threonine, and proline residues. As shown in the graphs of FIGS. 7A-7B, the peptide hydrogels and peptides are thermostable as a function of temperature.

In certain embodiments, the preparation and/or peptide may be mechanically stable. For instance, the preparation may be shear thinned or sonicated. The preparation may be sonicated without substantial degradation, loss of biological or chemical activity. The preparation may be shear thinned without substantial degradation, loss of biological or chemical activity.

In certain embodiments, the preparation and/or peptide may be sterile or sterilized. The preparation and/or peptide may be sterilized by autoclave sterilization. During autoclave sterilization, the preparation and/or peptide may be heated to a temperature of between 120° C. to 150° C., for example, up to 125° C., up to 135° C., or up to 150° C. The preparation and/or peptide may be held at autoclave temperature for at least about 2 minutes, for example, between about 2-20 minutes or between about 10-160 minutes. The autoclave sterilization may be sufficient to sterilize at least about 90%, 95%, 99%, 99.9%, 99.99%, 99.999%, or 100% of any pathogenic microorganism. The preparation and/or peptide may remain stable during and after autoclave sterilization. For instance, the preparation and/or peptide may remain physically, chemically, biologically, and/or functionally stable after autoclave sterilization.

In certain embodiments, the preparation and/or peptide may be pasteurized. During pasteurization, the preparation and/or peptide may be heated to a temperature of between 50° C. to 100° C., for example, up to 60° C., up to 70° C., or up to 100° C. The preparation and/or peptide may be held at pasteurization temperature for at least about 15 seconds, for example, between about 1-30 minutes or between about 3-15 minutes. The pasteurization may be sufficient to sterilize at least about 90%, 95%, 99%, 99.9%, 99.99%, or 99.999% of any pathogenic microorganism.

In certain embodiments, the preparation may be sterilized by ultra-high temperature (UHT) or high temperature/short time (HTST) sterilization. During UHT or HTST sterilization, the preparation and/or peptide may be heated to a temperature of between 100° C. to 150° C., for example, up to 130° C., up to 140° C., or up to 150° C. The preparation and/or peptide may be held at UHT or HTST temperature for at least about 15 seconds, for example, between about less than 1 minute to about 6 minutes, for example, between about 2-4 minutes. The UHT or HTST sterilization may be sufficient to sterilize at least about 90%, 95%, 99%, 99.9%, 99.99%, or 99.999% of any pathogenic microorganism.

In certain embodiments, the sterilization or pasteurization may be terminal. Terminal sterilization or pasteurization may refer to treatment of the preparation in a sealed end-use package.

The preparation and/or peptide may be stable during and after heat treatment. As disclosed herein, stability during and after heat treatment, for example, autoclave sterilization, may refer to reduced or inhibited degradation, biological activity, and chemical activity. For instance, the preparation and/or peptide may be heat treated without degradation, a loss of biological activity, or a loss of chemical activity. Biological activity may refer to any bioactive property of the peptide disclosed herein. In some embodiments, biological activity may refer to antimicrobial activity. Chemical activity may refer to any chemical or physicochemical property of the peptide disclosed herein. In some embodiments, chemical activity may refer to the ability to self-assemble and or shear-thinning properties of the peptide disclosed herein. Thus, the preparation and/or peptide may be heat treated without loss of antimicrobial activity, self-assembly, or shear-thinning properties. In certain embodiments, heat treatment may enhance one or more biological activity or chemical activity of the peptide and/or preparation. For example, heat treatment may enhance antimicrobial activity, self-assembly, or shear-thinning properties of the peptide or preparation.

The preparation may be sterile. For example, the preparation may remain substantially sterile without the addition of a preservative. The preparation may be substantially sterile without gamma irradiation treatment. The preparation may be substantially sterile without electron beam treatment.

The preparation may have a predetermined shelf-life. “Shelf-life” may refer to the length of time for which the preparation may remain stable and/or maintain efficacy after storage under the given conditions. The preparation and/or hydrogel may have a shelf-life of at least about 1 year at a temperature between −20° C. and 150° C. For instance, the preparation and/or hydrogel may have a shelf-life of at least about 1 year at room temperature (between about 20° C. and 25° C.). The preparation and/or hydrogel may have a shelf-life of at least about 2 years, about 3 years, about 4 years, about 5 years, or about 6 years at room temperature. The preparation and/or hydrogel may be stable at a pressure of up to about 25 psi, for example, up to about 15 psi.

The peptide may be capable of self-assembly at a temperature between 2° C. and 40° C. For example, the peptide may be capable of self-assembly in an environment having a temperature between 2° C. and 20° C., between 20° C. and 25° C., or between 36° C. and 40° C.

The peptide may be substantially unassembled at temperatures higher than 40° C. For instance, the peptide preparation may be substantially liquid at temperatures between 40° C. and 150° C. The peptide preparation may be substantially liquid and thermally stable at temperatures between 40° C. and 125° C. or up to 150° C. Temperature may be controlled for handling of the preparation. For example, the preparation may be heated to a temperature greater than 40° C. for packaging, handling, and/or administration in a liquid state.

The preparation may be formulated for a desired route of administration. For example, the preparation may be formulated for topical or parenteral administration. In particular, the preparation may be engineered to have a viscosity appropriate for topical administration or parenteral administration. Preparations for topical administration may be formulated to withstand environmental and mechanical stressors at the site of administration or target site. Preparations for parenteral administration may be formulated to reduce migration from the site of administration or target site. In other embodiments, preparations for parenteral administration may be formulated to trigger migration from a site of administration to the target site. The preparation may be formulated for administration by a particular delivery device. For example, the preparation may be formulated for administration by spray, dropper, or syringe. The preparation may be formulated for administration by injection or catheter.

Table 1 includes the analytical characterization of three exemplary peptide preparation samples. The exemplary peptides have arginine-rich sequences comprising two lysine amino acid residues. The values were detected by conventional detection methods. Components indicated “N.D.” were below detection limit. Peptide purification, residual solvents, peptide content, and water content, may be selected to control antimicrobial activity and cell membrane disruption potential of the hydrogels.

TABLE 1
Exemplary Peptide Preparations

| | | | | | | Residual Solvents | | | | | | | | |
|---|---|---|---|---|---|---|---|---|---|---|---|---|---|---|
| | | | | | | | | | | N,N- | | | | |
| | | | | | Net | | | | Triethyl | Dimethyl | Ethyl | | | |
| | Purity | Acetate | TFA | Water | Peptide | Isopropanol | Methanol | Acetonitrile | amine | formamide | ether | | | |
| # | (%) | (%) | (%) | (%) | (%) | (ppm) | (ppm) | (ppm) | (ppm) | (ppm) | (ppm) | DCM | DIC | MPS |
| 1 | 97.9 | 19.3 | N.D. | 16.5 | 78.6 | 81 | 28 | 17 | N.D. | N.D. | N.D. | N.D. | N.D. | N.D. |
| 2 | 93.7 | 16.3 | N.D. | 7.1 | 79.3 | 63 | 28 | 18 | 56 | 116 | N.D. | N.D. | N.D. | N.D. |
| 3 | 96.0 | 16.9 | N.D. | 6.2 | 76.1 | N.D. | N.D. | 4.6 | 2.9 | N.D. | N.D. | N.D. | N.D. | N.D. |

The purified peptide and hydrogel may be substantially endotoxin free without addition of a preservative or sterilization, as shown in Table 2. Thus, in some embodiments, the peptide preparation may be substantially free of a preservative.

TABLE 2
Endotoxin Levels of Different Compositions

| | OD at 410 nm | Endotoxin level (EU/mL) |
|---|---|---|
| Endotoxin free water | −0.000333333 | −0.016148109 |
| RO water | 3.436666667 | 1.267607913 |
| Tap water | 3.463666667 | 1.2776927 |
| Buffer solution | −0.056 | −0.036940201 |
| Peptide | 0.006 | −0.013782542 |
| Peptide hydrogel | 0.015 | −0.010420946 |

### The Self-Assembling Peptide Hydrogel

The preparations disclosed herein may be provided to self-assemble into a hydrogel having preselected properties. The polymeric hydrogel may have a substantially physiological pH. In general, the polymeric hydrogel may have a pH of between 4.0 and 9.0, for example, between 7.0 and 8.0, between 7.2 and 7.8, or between 7.3 and 7.5.

The polymeric hydrogel may be substantially transparent. For example, the polymeric hydrogel may be substantially free of turbidity, for example, visible turbidity. Visible turbidity may be determined by macroscopic and microscopic optical imaging. The polymeric hydrogel may be substantially free of peptide aggregates (peptide clusters), for example, visible peptide aggregates. Visible peptide aggregates may be determined by static light scattering (SLS) and UV-VIS testing. “Transparency” may refer to the hydrogel's ability to pass visible light. The substantially transparent hydrogel may have UV-VIS light absorbance of between about 0.1 to 3.0±1.5 at a wavelength of between about 205 nm to about 300 nm.

The assembled polymeric hydrogel may have a nano-porous structure. The polymeric hydrogel may be hydrated or substantially saturated. In some embodiments, the hydrogel may have between 90% w/v and 99.9% w/v aqueous solution, for example, between 92% w/v and 99.9% w/v or between 94% w/v and 99.9% w/v. The nano-porous structure may be selected to be impermeable to a target microorganism. Thus, the hydrogel may be used to encapsulate a target microorganism or to maintain the target site free from the target microorganism. The nano-porous structure may be selected to allow gaseous exchange at the target site. The polymeric hydrogel may have a nano-porous structure having a pore size of between 1 nm and 1000 nm, as selected (e.g., based on a target microorganism, target cell, or desired functionality). The polymeric hydrogel may have a fibril width of between 1 nm and 100 nm, as selected.

The hydrogel may generally be cationic in nature. In other embodiments, the hydrogel may be anionic in nature. In yet other embodiments, the hydrogel may be blended to contain multi-domains of cationic and/or anionic components. The hydrogel may be designed to have a preselected charge. The self-assembling peptide hydrogel disclosed herein may be tunable to biological functionality that supports the viability and function of transplanted therapeutic cells, to exhibit shear-thinning mechanical properties that allow easy and rapid administration in an intra-operative setting, to exhibit antimicrobial properties to control wound bioburden, to exhibit antiviral properties to treat or inhibit viral infection, and/or to exhibit antifungal properties to treat or inhibit fungal infection.

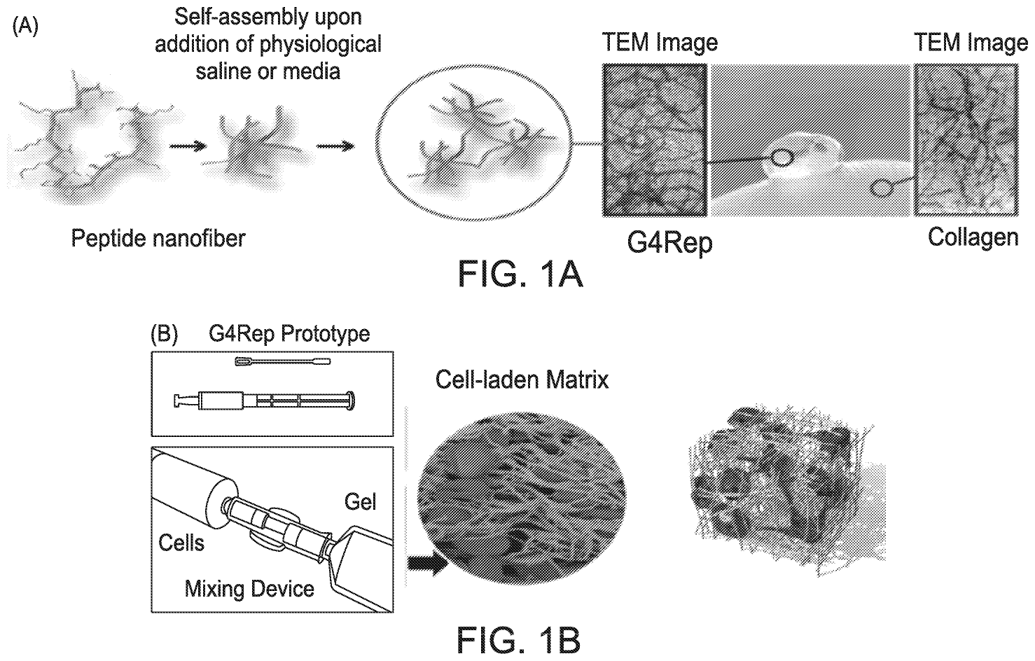

In particular, the peptide sequence and structure may include peptide functional groups that form nanofibers, which further self-assemble to form macromolecular structures (FIG. 1A-1B). The peptides may self-assemble in response to an environmental stimulus. The peptides may self-assemble in the presence of substantially physiological buffers, such as media or saline. The peptide hydrogels may assemble into an extracellular scaffolding matrix that is similar to native fibrillar collagen (FIG. 1A-1B). Schematics of gel matrix self-assembly and an exemplary nanostructure are shown in FIGS. 1A-1B. As shown in FIG. 1A, single peptide nanofibers self-assemble into a gel when ionic buffer is added. FIG. 1A includes a TEM image demonstrating that the nanostructure and pore size of the peptide gel look similar to native ECM (collagen). FIG. 1B includes a schematic drawing of an intra-operative mixing device for mixing a cell suspension with peptide gel matrix. A schematic SEM image in FIG. 1B of the cell-laden matrix demonstrates the exemplary concept of cells in matrix.

The peptide may be engineered by design to self-assemble into a hydrogel which is substantially biocompatible. The peptide may be engineered by design to self-assemble into a hydrogel that is cell friendly. In certain embodiments, the cell-friendly polymeric hydrogel may be non-inflammatory, and/or non-toxic. The cell-friendly polymeric hydrogel may be substantially biodegradable. The peptide may be engineered by design to be substantially antimicrobial, antiviral, and/or antifungal.

The short peptides and/or peptide functional groups may be produced synthetically. Thus, the peptides may provide ease of manufacturing, scale-up, and quality control. In general, the peptides may be manufactured without the use of plant or animal expression systems. The peptides may be substantially free of naturally occurring endotoxins and disease-transmitting pathogens. In addition, the peptide sequence and functional groups may be tuned, allowing a versatility in control and design of the assembled hydrogel, including with respect to physical and chemical properties, such as biodegradation, mechanical properties, and biological activity.

The peptide may have a functional group engineered for a target indication. For instance, the peptide may have a bioactive functional group. The target indication may be tissue engineering of a target tissue. The target indication may include, for example, cell culture, cell delivery, wound healing, and/or treatment of biofilm. Thus, the peptide may be engineered by design to self-assemble into a hydrogel which is substantially biocompatible. The peptide functional group may have between about 3 and about 30 amino acid residues. For example, the peptide functional group may have between about 3 and about 20 amino acid residues. The peptide functional group may have a sequence selected from RGD, IKVAV, YIGSR, LKKTETQ, SNKPGVL, PKPQQFFGLM, GKLTWQELYQLKYKGI, and GGG.

In some embodiments, the peptide may include a modification selected from a linker and a spacer. Peptide “linkers” may generally refer to short amino acid sequences included to link multiple domains of the peptide. Peptide “spacers” may generally refer to amino acid sequences positioned to link and control the spatial relationship of the multiple domains of the assembled protein. The linker or spacer may be hydrophobic or hydrophilic. The linker or spacer may be rigid or flexible. Exemplary spacers include aminohexanoic acid (Ahx) (hydrophobic) and poly (ethylene) glycol (PEG) (hydrophilic). Glycine rich spacers are generally flexible.

Exemplary bioactive functional groups include laminin, bone marrow homing, collagen (e.g., I, II, and VI), bone marrow purification, and RGD/fibronectin types. Exemplary bioactive functional groups include VEGF, Substance P, Thymosin Beta, Cardiac Homing Peptide, Bone Marrow Homing Peptide, Osteopontin, and Ostegenic peptide. Exemplary bioactive functional groups include those in Tables 3-5 below.

TABLE 3
Exemplary Bioactive Functional Groups

| ECM Motif | ECM Type |
|---|---|
| -GGPDSGR | Laminin |
| -GGSDPGYIGSR | Laminin |
| -GGSKPPGTSS | Bone marrow homing |
| -GGDGEA | Collagen II |
| -GGPFSSTKT | Bone marrow homing |
| -GGFLGFPT | Bone marrow purification |
| -GGRGDS | RGD/fibronectin |
| -GGIKVAV | Laminin |
| -GGFPDERGVEGPGP | Collagen I |
| -GGPRGDSGYRGDSF | Collagen VI |

TABLE 4
Exemplary Bioactive Functional Groups

| Protein/Peptide | Bioactive Motif |
|---|---|
| VEGF | GKLTWQELYQLKYKGI |
| Substance P | RPKPQQFFGLM |
| Thymosin Beta | SNKPG |
| Thymosin Beta | LKKTETQG |
| Cardiac Homing Peptide | CSTMLKAC |
| Bone Marrow Homing Peptide | GCPFSSTKTE |
| Osteopontin | TLEGTKKGHKLHLDY |
| Osteogenic Peptide | ALKRQGRTLYGFGG |

TABLE 5
Exemplary Bioactive Functional Groups

| Peptide name | Sequence | Source |
|---|---|---|
| RGD | XRGDX | Fibronectin, osteopontin, |
| | | BSP and others |
| pRGD/bsp-RGD | CGGNGEPRGDTYRAY | BSP |
| hBSP₂₇₈₋₂₉₃ | YESENGEPRGDNYRAY | BSP |
| PHSRN | PHSRN | Fibronectin |
| F105 | YKRSRYT | FGF-2 |
| F119 | KRTGQYKLGSKTGPGQK | FGF-2 |
| F36 | PDGRVD | FGF-2 |
| F77 | KEDGRLL | FGF-2 |
| GFOGER | GGYGGGPC(GPP)₅GFOGER | Collagen type I |
| | (GPP)₅GPC | |
| DGEA | DGEA | Collagen type I |
| P15 | GTPGPQGIAGQRGVV | Collagen type I |
| AC-100 | ERGDNDISPFSGDGQ | MEPE |
| ODP (osteopontin- | DVDVPDGRGDSLAYG | Osteopontin |
| derived peptide) | | |
| RRETAWA | RRETAWA | Identified by phage display |
| YIGSR | YIGSR | Laminin |
| IKVAV | IKVAV | Laminin |
| Ln2-P3 | DLTIDDSYWYRI | Laminin |
| KRSR | KRSR | BSP, fibronectin, |
| | | osteopontin and others |
| Consensus heparin | FHRRIKA | BSP |
| binding domain | | |
| HBD peptide | RKKNPNCRRH | BMP-4 |
| BMP-2-derived peptide | NSVNSKIPKACCVPTELSAI | BMP-2 |
| BMP-2-derived peptide | KIPKASSVPTELSAISTLYL | BMP-2 |
| P24 | S^{[PO}₄^{]}KIPKASSVPTELSAIST | BMP-2 |
| | LYLDDD | |
| pBMP-9/SpBMP-9 | CGGKVGKA(CC/SS)VPTKL | BMP-9 |
| | SPISVLYK | |
| BMP-9-derived peptide | RKVGKASSVPTKLSPISILYK | BMP-9 |
| BMP-7-derived peptide | RTVPKPSSAPTQLNAISTLYF | BMP-7 |
| BFP-1 | GQGFSYPYKAVFSTQ | BMP-7 |
| OPD (osteopromotive- | DWIVA | BMP-2 |
| domain) | | |
| CBM | GLRSKSKKFRRPDIQYPDA | Osteopontin |
| | TDEDITSHM | |
| BCSP ™-1 | NGLPGPIGP | Collagen type I |
| OGP (osteogenic growth | ALKRQGRTLYGFGG | Naturally occurring |
| peptide) | | |
| OGP₁₀₋₁₄ | YGFGG | OGP |
| PTH₁₋₃₄ | SVSEIQLMHNLGKHLNSME | Parathyroid hormone |
| | RVEWLRKKLQDVHNF | |
| CB | NGVFKYRRRYYLYKHAYF | BSP |
| | YPHLKRFPVQ | |
| CTC peptide | IAGVGGEKSGGF | Collagen-IIIa |
| Substance-P | RPKPQQFFGLM | Naturally occurring |
| Endothelin-1 | CSCSSLMDKECVYFCHLD | Naturally occurring |
| | IIW | |
| QK | KLTWQELYQLKYKGI | VEGF |
| PBA2-1c | (CVRKIEIVRKK)₂-Ahx-Ahx- | PDGF |
| | Ahx-RKRKLERIAR | |
| Exendin 4 | HGEGTFTSDLSKQMEEEAVR | Glucagon-like peptide |
| | LFIEWLKNGGPSSGAPPPS | |
| OPD (osteopontin- | SVVYGLR | Osteopontin |
| derived peptide) | | |
| SPARC 113 | TLEGTKKGHKLHLDY | Osteonectin |
| SPARC 118 | KKGHK | Osteonectin |
| TP508 | AGYKPDEGKRGDACEGDS | Thrombin |
| | GGPFV | |
| RoY | YPHIDSLGHWRR | Designed |
| LL-37 | LLGDFFRKSKEKIGKEFKRI | hCAP18 |
| | VQRIKDFLRNLVPRTES | |

The peptide may have a functional group engineered to control or alter charge or pH of the peptide or preparation. A pre-selected charge or pH may provide bioactive properties. In some embodiments, a pre-selected charge or pH may provide antimicrobial, antifungal, and/or antiviral properties. In some embodiments, a pre-selected charge or pH may allow the preparation to be administered to a compatible target site.

The peptide may have an antimicrobial functional group. The antimicrobial functional group may include a conserved sequence of antimicrobial residues. In other embodiments, the antimicrobial functional group may overlap or partially overlap with the self-assembling functional group. In at least one embodiment, the peptide may have alternating or substantially alternating antimicrobial and self-assembling residues.

The peptide may have an antifungal functional group. The antifungal functional group may include a conserved sequence of antifungal residues. In other embodiments, the antifungal functional group may overlap or partially overlap with the self-assembling functional group. In at least one embodiment, the peptide may have alternating or substantially alternating antifungal and self-assembling residues.

The peptide may have an antiviral functional group. The antiviral functional group may include a conserved sequence of antiviral residues. In other embodiments, the antiviral functional group may overlap or partially overlap with the self-assembling functional group. In at least one embodiment, the peptide may have alternating or substantially alternating antiviral and self-assembling residues.

The self-assembled hydrogel may be designed to have cell protective properties at the target site. In particular, the self-assembled hydrogel may be designed to be protective against foreign microorganisms, e.g., pathogenic microorganisms. The self-assembled hydrogel may be designed to be protective against fungal organisms. The self-assembled hydrogel may be designed to be protective against immune attack from environmental immune cells. The antimicrobial, antiviral, antifungal, and/or protective properties of the hydrogel may not substantially affect the viability, growth, or function of cells at the target site.

The protective properties of the hydrogel may be engineered by altering the net charge of the peptides. In some embodiments, the net charge may be altered by controlling one or more of the presence or absence of cationic particles or peptides, the presence or absence of anionic particles or peptides, buffers, salts, peptide concentration, peptide purity, and the presence or absence of peptide counterions. The peptide may be engineered to have positively charged, negatively charged, hydrophobic, or hydrophilic amino acid residues. In an exemplary embodiment, the peptide may provide antimicrobial, antiviral, and/or antifungal properties by inclusion amino acids which are positively charged at a substantially neutral pH level. Such amino acids may include, for example, arginine, lysine, tryptophan, and histidine.

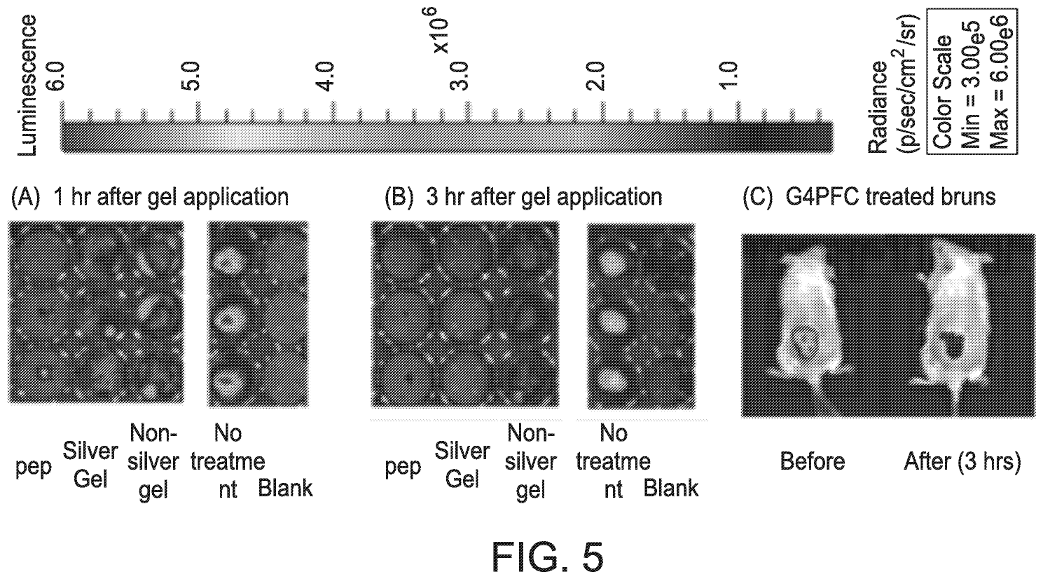

The peptide hydrogel may additionally exhibit antimicrobial properties. In general, the antimicrobial properties may be provided by including an antimicrobial functional group. In some embodiments, the antimicrobial functional group may include a cation-rich peptide sequence. In exemplary embodiments, the antimicrobial functional group may include varying ratios of lysine (K) and arginine (R) (FIG. 4). The antimicrobial peptide hydrogel may provide antimicrobial effects against gram-positive and negative bacteria, including, for example, E. coli (FIG. 4), S. aureus, and P. aeruginosa. FIG. 4 is a graph showing antimicrobial activity (as percent non-viable E. coli remaining after 24 hours of administration) of varying concentrations of peptides having 8 arginine residues (PEP8R), 6 arginine residues (PEP6R), 4 arginine residues (PEP4R), and 2 arginine residues (PEP2R).

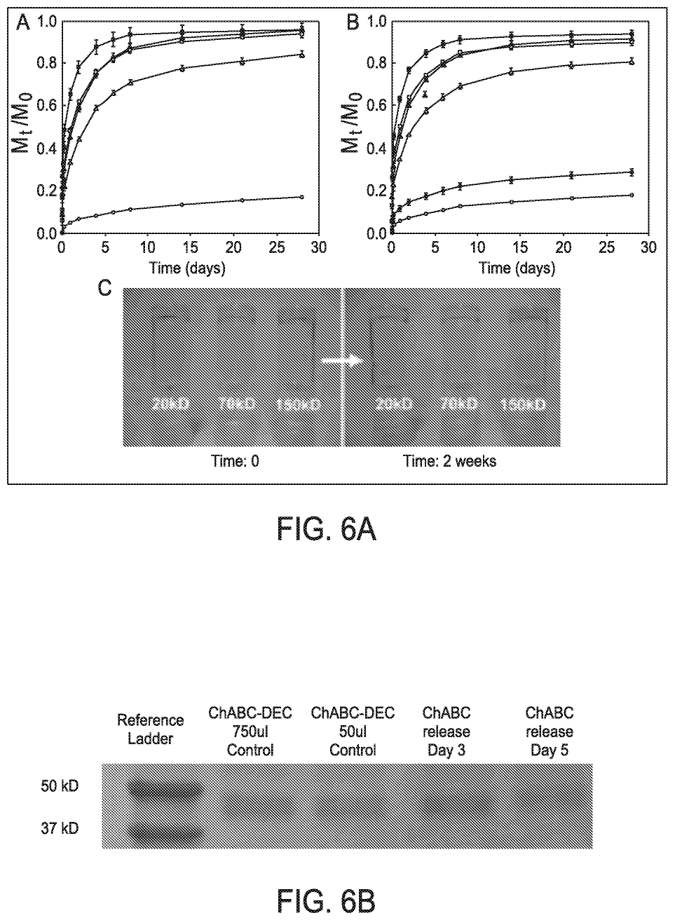

The peptide hydrogels may exhibit broad spectrum antimicrobial activity. In accordance with certain embodiments, the peptide hydrogels may reduce bioburden in vivo in partial thickness wounds inoculated with methicillin-resistant S. aureus (MRSA) (FIG. 5). FIG. 5 shows preliminary data demonstrating antimicrobial benefits of treating bioluminescent MRSA (US300) with peptide gels. Images (A) and (B) show wells plated with 100 μl of gel and 100 μl of US300 (1×10⁸ CFU/ml) demonstrating the antimicrobial activity of peptide gels compared to controls at 1 hour and 3 hours (n=3). Image (C) shows mice with partial thickness burns inoculated with 50 μl of 10⁸ CFU/ml US300 and treated with peptide gels. As shown in image (C), the mice exhibit reduced bioburden at 3 hours after administration.

In particular, the peptide hydrogel may exhibit antimicrobial properties against foreign and/or pathogenic microorganisms, and be compatible with the administered cells. For example, such peptide hydrogels may be compatible with mammalian erythrocytes and macrophages. In one exemplary experiment, when bacteria and mammalian cells were seeded simultaneously onto the peptide hydrogels disclosed herein, the bacteria were killed while the mammalian cells remained >90% viable after 24 hours and could continue to proliferate.

In some embodiments, the peptides may include functional groups to enhance or promote biological activity compatible or synergizing with MSC function. For example, in certain embodiments, the peptide sequence may contain a functional group that mimics fibronectin and promotes adhesion and proliferation of human MSCs to a greater extent than other ECM ligands. In certain embodiments, the peptide sequence may contain a functional group comprising a neuropeptide to promote diabetic wound healing by suppressing inflammation and inducing angiogenesis. In certain embodiments, the peptide sequence may contain a functional group comprising a neuropeptide to induce the proliferation and migration of MSCs, as well as enhance the immunomodulatory function of MSCs. In certain embodiments, the peptide sequence may contain a functional group to improve wound healing by increasing angiogenesis and inducing MSC proliferation and migration. In certain embodiments, the peptide sequence may lack a functional group that inhibits proteolytic activity. The peptide may be engineered to contain other functional groups known to one of skill in the art.

In vitro, the peptide hydrogels disclosed herein may allow cell invasion and proliferation in 3D constructs, allowing the hydrogels to serve as scaffolding matrices for tissue regeneration. The peptide hydrogels may show biocompatibility following subcutaneous implantation. Experiments show minimal cell debris or dead cells at the gel implantation site 7 days post-subcutaneous administration. Experiments further show minimal increases in cytokine concentration in the gel and surrounding tissues compared to naïve tissues, suggesting the gel has insignificant acute inflammation effects.

### Kits Comprising the Peptide Preparation

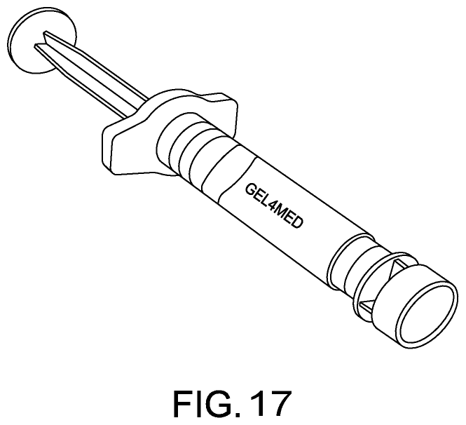

Kits comprising the peptide preparation are described herein. The kit may comprise the peptide preparation and a buffer solution. The buffer may be configured to induce self-assembly of the peptide prior to or concurrently with administration of the peptide. Each of the peptide preparation and the buffer may be included in a vial or chamber. For example, the kit may comprise a pre-filled packaging containing one or more of the preparation and the buffer. The kit may comprise one or more devices for use and/or delivery of the peptide preparation. The kit may comprise a mixing device. The kit may comprise a delivery device. In certain embodiments, the delivery device and/or mixing device may be the pre-filled packaging, for example, the kit may comprise a pre-filled syringe, spray bottle, ampule, or tube. FIG. 17 is a photograph of the preparation packaged in an end-use container. The exemplary end-use container of FIG. 17 is a pre-filled syringe. The end-use container may be employed as a delivery device or a mixing device. The kit and/or any component of the kit may be sterile or sterilized. For example, the kit and/or any component may be sterilized using autoclave sterilization, optionally terminal autoclave sterilization.

Any one or more component of the kit may be autoclavable. The packaged kit may be autoclavable. Any one or more component of the kit may be sterilized or sterile. For example, any one or more component of the kit may be sterilized by autoclave. The sterilized one or more component may be packaged in a substantially air-tight container. In some embodiments, the packaged kit may be sterilized, e.g., by autoclave.

In certain embodiments, the kit may comprise the purified peptide in a dried or powder form. For example, the purified peptide may be lyophilized. The kit may comprise a biocompatible solution to be combined with the purified peptide to produce the peptide preparation. In other embodiments, the kit may comprise instructions to combine the purified peptide with a biocompatible solution to produce the preparation. The kit may additionally comprise the buffer solution.

The kit may comprise instructions for use. In particular, the kit may comprise instructions to combine the buffer with the preparation, optionally in the mixing device, to form the hydrogel. A user may be instructed to combine the preparation and the buffer at the point of use. In some embodiments, the user may be instructed to combine the preparation and the buffer prior to administration or concurrently with administration. The user may be instructed to apply the preparation and the buffer to the target site separately.

The kit may additionally comprise instructions to store the kit under recommended storing conditions. For instance, the kit may comprise instructions to store the preparation or any component at room temperature (approximately 20-25° C.). The kit may comprise instructions to store the preparation or any component under refrigeration temperature (approximately 1-4° C.). The kit may comprise instructions to store the preparation or any component under freezer temperature (approximately 0 to −20° C.). The kit may comprise instructions to store the preparation or any component at body temperature (approximately 36-38° C.). The kit may comprise instructions to store the preparation or any component under cool and dry conditions.

The kit may additionally comprise an indication of expiration for the preparation or any component. The indication of expiration may be about 1 year after packaging. The indication of expiration may be between about 6 months and about 10 years after packaging, for example, between about 1 year and about 5 years after packaging.

The kit may comprise additional components for administration in combination with the preparation. In some embodiments, the kit may comprise instructions to combine the additional component prior to administration or concurrently with administration. The kit may comprise instructions to administer the preparation and the additional component to the target site separately. The additional component may be or comprise an antibacterial formulation, an antiviral formulation, an antifungal formulation, an anti-tumor formulation, an anti-inflammatory formulation, a cell culture media, a cell culture serum, an anti-odor formulation, an analgesic, a hemostat formulation, local anesthetic, or a pain-relief formulation. The additional component may be or comprise an anti-scarring agent and/or an axon regeneration agent. In particular embodiments, the kit may comprise biological material, for example, cells, cell-derived materials, tissue, and/or tissue-derived materials. The biological material may comprise a culture of cells for administration in combination with the preparation, as described herein. In some embodiments, the kit may further comprise a dressing, e.g., a topical dressing, a barrier dressing, and/or a wound dressing.

The kit may comprise one or more component configured to induce shear-thinning of the hydrogel. Mixing devices or delivery devices (described below) may be employed to induce shear-thinning of the hydrogel by mechanical agitation. The kit may comprise one or more component selected from a temperature control device, a pH control additive, an ion chelator composition, a solvent, a sound control device, a lyophilization device, and an air drying device to induce shear-thinning. For example, the kit may comprise a heater or cooler, a source of an acid or a base, a source of an ion chelator, a source of a solvent, a speaker or sound transmitter, a lyophilizer, or a compressed air dryer, or a fan.

### Mixing Devices

Mixing devices for preparation of a hydrogel at a point of care are disclosed herein. The device may be a multi-chamber device. In exemplary embodiments, the device may be a two-chamber device. The devices may include a first chamber for a peptide preparation. The preparation may comprise a self-assembling peptide in a biocompatible solution. The devices may include a second chamber for a buffer solution. The first chamber and the second chamber may be separated by a barrier provided to prevent fluid communication between the first chamber and the second chamber. The devices may, optionally, further comprise a mixing chamber. The mixing chamber may be fluidly connectable to the first chamber and the second chamber. Prior to mixing, the mixing chamber may be separated from the first chamber and/or the second chamber by a barrier. In other embodiments, the mixing device may be configured for direct mixing of the contents of the first and second chambers. In some embodiments, the devices may comprise a third chamber for an additional formulation or compound to be administered to the subject. The third chamber may be separated from the first chamber, the second chamber, and/or the mixing chamber. The third chamber may be fluidly connectable to the first chamber and/or the second chamber directly or via the mixing chamber.

The device may be a single use device. The device may be a multiple use device.

In an exemplary embodiment, each of the first, second, or third chamber may be a syringe barrel. Each barrel may have an associated plunger for agitation. Each barrel may be hermetically fitted to a coupling adapter, which forms the mixing chamber. The hermetic fitting may be, for example, a luer lock or luer taper connection.

The preparation and buffer may be agitated or otherwise mixed to form a homogenous or substantially homogenous mixture, inducing hydrogelation. In some embodiments, the preparation and buffer may be agitated by transferring the mixture back and forth between the first chamber and the second chamber. In some embodiments, the hydrogel exhibits shear-thinning properties, such that during agitation the mixture is substantially liquid. Upon settling, the mixture may form a solid or gel material.

In exemplary embodiments, the device may be configured to prepare a cell graft at a point of use. In use, the first chamber may comprise the cell preparation and the second chamber may comprise the peptide preparation. The cell preparation may comprise buffer. Alternatively, a third chamber may comprise buffer. Upon actuation the cell preparation and the peptide preparation may mix or contact, i.e. in the mixing chamber. The cells may be suspended in the peptide solution, forming a cell suspension comprising self-assembling peptides.

The cell preparation and the peptide preparation may be mixed with buffer, forming a buffer suspension. The buffer suspension may be agitated as described above, inducing self-assembly of the hydrogel. The buffer suspension may be agitated to disperse the cells, forming a homogenous or substantially homogenous mixture. The homogenous or substantially homogenous suspension may self-assemble to form a hydrogel cell graft.

The mixing device may be a static mixing device. A static mixer may generally comprise a device for substantially continuous mixing of the preparation without moving components. For example, the static mixer may comprise a cylindrical or rectangular housing with one or more inlet for each component to be mixed and a single outlet for the mixture. The static mixer may comprise a plate-type mixer.

The mixing device may generally be formed or lined with an inert, thermally-stable material. In certain embodiments, the material may be opaque and/or shatter resistant.

### Delivery Devices

In some embodiments, the kits may include a delivery device. For instance, the kits may include a syringe or catheter. The kits may include a dropper. The kits may include a spray, e.g. in conjunction with a bottle. The spray device may be, for example, a nasal spray. The kits may include a tube or ampule. The kits may include a film, for example. The type of delivery device may be selected based on a target indication. Additionally, the properties of the delivery device may be selected based on a target indication. For instance, a syringe for topical delivery of the preparation may have a larger passage than a syringe for injection of the preparation.

In some embodiments, the syringe may be used for topical application of the preparation. In other embodiments, the syringe may comprise a needle for parenteral application. The needle of the syringe may have a Birmingham system gauge between 7 and 34. The catheter may be used for parenteral application. The needle of the catheter may have a Birmingham system gauge between 14 and 26. The peptide may be formulated for administration through a particular gauge needle. For instance, the peptide may be selected to have a variable viscosity that will allow application of the preparation through a particular gauge needle.

In some embodiments, the spray bottle may be used for topical application of the preparation. The spray bottle may comprise a container for the preparation and a spray nozzle. The spray nozzle may be configured for targeted delivery to a target tissue. For instance, a spray nozzle for targeted delivery to an epithelial tissue may have a substantially flat surface and a spray nozzle for targeted delivery to an intranasal tissue may have a substantially conical surface. The spray nozzle may be configured to deliver a predetermined amount of the preparation. In some embodiments, the spray nozzle may be configured to deliver the preparation in substantially unidirectional flow, optionally, regardless of orientation of the spray bottle.

The spray nozzle may be configured to reduce retrograde flow. In certain embodiments, the spray nozzle may be spring-loaded. In other embodiments, the spray nozzle may be pressure actuated. The actuation pressure may be selected based on the variable viscosity of the preparation. For instance, the actuation pressure may be selected to be sufficient to dispense the hydrogel through the spray nozzle.

The film may be used for topical application of the preparation. The film may be saturated with the preparation. The film may be used as a barrier dressing and/or hemostat. In some embodiments, the film may accompany a barrier dressing.

The delivery device may be a single use device. The delivery device may be a multiple use device. The delivery device may include a first chamber for a peptide preparation. The preparation may comprise a self-assembling peptide in a biocompatible solution. The delivery device may include a second chamber for a buffer solution. The first chamber and the second chamber may be separated by a barrier provided to prevent fluid communication between the first chamber and the second chamber. The delivery device may be constructed and arranged for administration of the peptide preparation and the buffer solution simultaneously or substantially simultaneously. In some embodiments, the delivery device may comprise a third chamber for an additional formulation or compound to be administered to the subject. The third chamber may be separated from the first chamber and/or the second chamber.

The delivery device may generally be formed or lined with an inert, thermally-stable material. In certain embodiments, the material may be opaque and/or shatter resistant.

### Coated Medical or Surgical Devices

In some embodiments, medical or surgical tools may have at least a portion of an exterior surface coated with the preparations or hydrogels disclosed herein. The coating may enable the exterior surface of the tool to exhibit antimicrobial properties, reducing incidence of infection. The coating may enable the exterior surface of the tool to be biocompatible or cytocompatible, reducing rejection and adverse reaction from contact.

The surgical tool may be a surgical instrument. For example, the tool may be a grasper, such as forceps, clamp or occluder, needle driver or needle holder, a suture or suture needle, retractor, distractor, positioner, stereotactic device, mechanical cutter, such as scalpel, lancet, drill bit, rasp, trocar, ligasure, harmonic scalpel, surgical scissors, or rongeur, dilator, specula, suction tip or tube, sealing device, such as surgical stapler, irrigation or injection needle, tip and tube, powered device, such as drill, cranial drill, or dermatome, scopes or probe, including fiber optic endoscope and tactile probe, carrier or applier for optical, electronic, and mechanical devices, ultrasound tissue disruptor, cryotome, cutting laser guide, or a measurement device, such as ruler or caliper. Other surgical tools or instruments are within the scope of the disclosure.

The medical or surgical tool may be an implantable tool. For example, the medical or surgical tool may be an implantable device, such as, implantable cardioverter defibrillator (ICD), pacemaker, intra-uterine device (IUD), stent, e.g., coronary stent, ear tube, or eye lens. Other implantable tools are within the scope of the disclosure. The implantable medical or surgical tool may be a prosthetic or a portion of a prosthetic device, for example, a prosthetic hip, knee, shoulder, or bone or a portion of a prosthetic limb. The implantable medical or surgical tool may be a mechanical tool, such as a screw, rod, pin, plate, disk, or other mechanical support. The implantable medical or surgical tool may be a cosmetic tool, such as breast implant, calf implant, buttock implant, chin implant, cheekbone implant, or other plastic or reconstructive surgery implant. Other medical or implantable tools are within the scope of the disclosure.

The formulation and/or thickness of the coating may be selected to be temporary, for example, degrading within a pre-determined period of time, for example, less than about 3 months, less than about 1 month, or less than about 2 weeks. The formulation and/or thickness of the coating may be selected to be semi-permanent, for example, degrading within a predetermined period of time of between about 3 months and 3 years, or between about 6 months and 2 years. The formulation and/or thickness of the coating may be selected to be permanent, for example, having a lifespan of more than 2 years or more than 3 years, or having a lifespan longer than the predetermined period of time that the medical or surgical tool is in contact with the subject.

### Methods of Treatment by Administration of Peptide Hydrogels

In some embodiments, the preparations disclosed herein may be administered according to a predetermined regimen. The preparations disclosed herein may be administered daily, weekly, monthly, yearly, or bi-yearly.

The preparations disclosed herein may provide immediate release effects. For example, the onset of action of the active ingredient may be less than one minute, several minutes, or less than one hour.

The preparations disclosed herein may provide delayed release effects. For example, the onset of action of the active ingredient may be more than one hour, several hours, more than one day, more than several days, or more than one week.

The preparations disclosed herein may provide extended release effects. For example, the preparations may be effective for more than one day, more than several days, more than one week, more than one month, several months, or up to about 6 months.

The preparations disclosed herein may be administered in conjunction with a medical approach that treats the relevant disease or disorder or a symptom of the relevant disease or disorder. For example, the preparations may be administered in conjunction with a medical approach that is approved to treat the relative disease or disorder or a symptom of the relevant disease or disorder. The preparations may be administered in conjunction with a medical approach that is commonly used to treat the relevant disease or disorder or a symptom of the relevant disease or disorder.

The preparations disclosed herein may be administered in combination with a surgical treatment. The preparations disclosed herein may be administered to treat wounds associated with the surgical treatment and/or to prevent or treat biofilm.

The preparations disclosed herein may be administered in combination with an anti-inflammatory agent or treatment. Anti-inflammatory agent may refer to a composition or treatment which reduces or inhibits local or systemic inflammation. The anti-inflammatory agent may comprise, e.g., non-steroidal anti-inflammatory drugs (NSAID), antileukotrienes, immune selective anti-inflammatory derivatives (ImSAID), and/or hypothermia treatment.

The preparations disclosed herein may be administered in combination with an antibacterial, antiviral, and/or antifungal agent. Such agents may refer to compositions or treatments which eliminate or inhibit proliferation of bacterial, viral, and/or fungal organisms, respectively. Exemplary antibacterial agents include antibiotics and topical antiseptics and disinfectants. The antiviral agent may be a target-specific antiviral agent or a broad-spectrum antiviral agent (e.g., remdesivir, ritonavir/lopinavir). Exemplary local antiviral agents include topical antiseptics and disinfectants. Exemplary antifungal agents include antifungal antibiotics, synthetic agents (e.g., flucytosine, azoles, allylamines, and echinocandins), and topical antiseptics and disinfectants.

The preparations disclosed herein may be administered to treat a wound, for example, an acute, a sub-acute, or a chronic wound. The wound may be a surgical wound, laceration, burn wound, bite/sting wound, vascular wound (venous, arterial or mixed), diabetic wound, neuropathic wound, pressure wound, ischemic wound, moisture-associated dermatitis, or result from a pathological process. In certain embodiments, the preparations may be administered in an amount effective to treat diabetic foot ulcers (DFU). In certain embodiments, the preparations may be administered in an amount effective to treat gastrointestinal wounds, such as anal fistulas, diverticulitis, and ulcers. In particular, the preparations may be administered in an amount effective to promote infection free wound closures.

The preparations disclosed herein may be administered in combination with a treatment or agent to provide anesthesia and/or pain-relief, e.g., local anesthetic. “Anesthetic” may refer to a composition which provides temporary loss of sensation or awareness. The anesthetic may be a general anesthetic (e.g., GABA receptor agonists, NMDA receptor antagonists, or two-pore potassium channel activators) or a local anesthetic (e.g., ester group agents, amide group agents, and naturally derived agents).

The preparations may be administered in combination with an analgesic or pain-relief agent. “Analgesic” may refer to a composition for systemic treatment or inhibition of pain. The analgesic may comprise an anti-inflammatory agent or an opioid.

The preparations disclosed herein may be administered in combination with a hemostat agent. “Hemostat” may refer to a tool or composition to control bleeding. Exemplary hemostat compositions include collagen-based agents, cellulose-based agents, and chitosan-based agents.

The preparations disclosed herein may be administered in combination with a treatment or agent to enhance cell or tissue graft therapy. In certain embodiments, the preparations disclosed herein may be administered in combination with a treatment or agent to prevent or inhibit cell death and/or control or reduce inflammation. The preparations disclosed herein may be administered in combination with cell culture media or cell culture serum.

The administered peptide hydrogels may have an immediate local effect. For instance, the administered preparations may provide localized wound healing or injury treatment effects by closing the wound or filling a void. In certain embodiments, the administered hydrogels may have a systemic effect. For instance, the administered hydrogels may enable cell migration or communication between cell grafts and environmental cells, resulting in a systemic effect. In other embodiments, the administered hydrogels may enable delivery of cell products or byproducts, resulting in a systemic effect. The administered peptide hydrogels may have antimicrobial, antiviral, and/or antifungal properties at a localized site of administration. In other embodiments, the administered peptide hydrogels may provide systemic antimicrobial, antiviral, and/or antifungal properties.

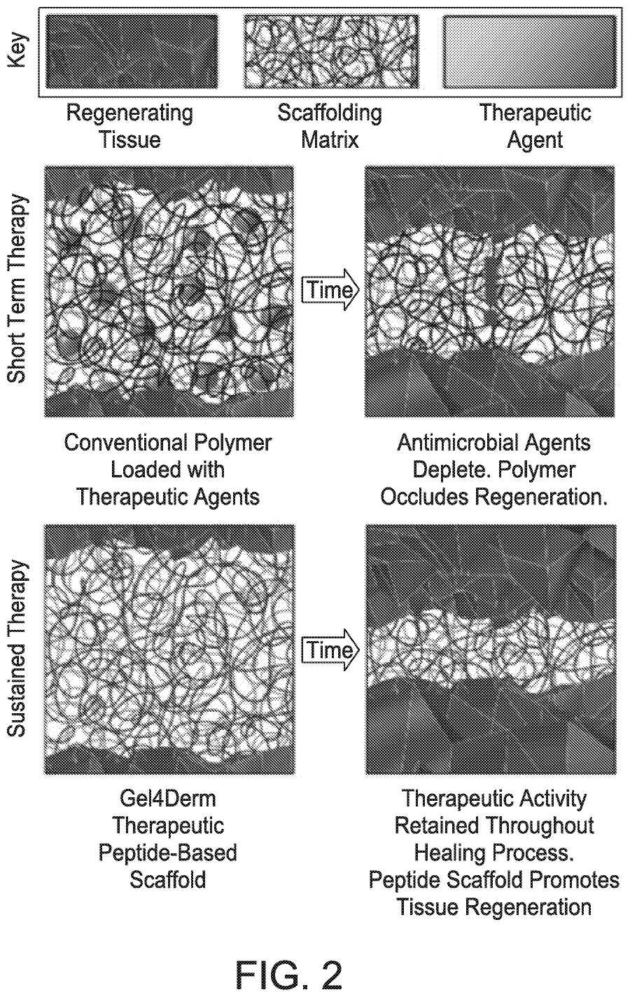

The administered peptide hydrogels may have long-term, sustained antimicrobial, antiviral, and/or antifungal properties at a localized site of administration. The peptide may be designed to form a hydrogel having a direct contact antimicrobial, antiviral, antifungal effect. Thus, the hydrogel may eradicate microorganisms which directly contact the hydrogel at the target site. The hydrogel may be substantially free of encapsulated antimicrobial, antiviral, and/or antifungal agents. Furthermore, the local antimicrobial, antiviral, and/or antifungal effect may persist as long as the hydrogel is in contact with the target tissue. FIG. 2 includes images which show sustained antimicrobial, antiviral, and/or antifungal effect at the target site.

To provide a systemic antimicrobial, antiviral, and/or antifungal effect, the peptide hydrogel may additionally comprise encapsulated antimicrobial, antiviral, and/or antifungal agents. Administration of such a hydrogel may generally provide: (1) local antimicrobial, antiviral, and/or antifungal treatment by direct contact as previously described, and (2) systemic antimicrobial, antiviral, and/or antifungal treatment as a vehicle of an encapsulated therapeutic agent.

The preparations disclosed herein may be formulated as a hemostat, debridement agent, or barrier dressing (e.g., antimicrobial, antifungal, or antiviral barrier dressing). The preparations may be formulated for wound treatment. Exemplary wounds which may be treated by administration of the preparation include partial and full thickness wounds (e.g., pressure sores, leg ulcers, diabetic ulcers), first and second degree burns, tunneled/undermined wounds, surgical wounds (e.g., associated with donor sites/grafts, tissue and cell grafts, Post-Moh's surgery, post laser surgery, podiatric, sound dehiscence), trauma wounds (e.g., abrasions, lacerations, burns, skin tears), gastrointestinal wounds (e.g., anal fistulas, diverticulitis, ulcers), and draining wounds. The preparations may be formulated for administration to a predetermined target tissue, for example, mesenchymal tissue, connective tissue, muscle tissue, nervous tissue, embryonic tissue, dermal tissue, bone tissue, dental tissue, corneal tissue, cutaneous tissue, integumental tissue, soft tissue, and hard tissue, or a biological fluid.

### Methods of Treatment of Microbial Infection

The preparation may be formulated to provide antimicrobial properties upon administration at a target site. For example, the self-assembled polymeric hydrogel may have antimicrobial properties. As disclosed herein, “antimicrobial” properties may refer to an effect against a microbial population, e.g., killing or inhibiting one or more microorganism from a microbial population. Thus, methods of treating a microbial infection or killing or inhibiting proliferation of a target microorganism are disclosed herein. “Proliferation” may generally refer to the metabolic or reproductive activity of an organism. Methods of reducing or eliminating a microbial contamination are disclosed herein. Methods of management of a microbial bioburden are disclosed herein. The methods may generally comprise administering the preparation in an amount effective to promote deactivation of a target microorganism. In particular, a preparation comprising about 3.0% w/v or less of the peptide, for example, 1.5% w/v or less, or 1.0% w/v or less, may provide antimicrobial properties at a target site.

The methods may comprise identifying a subject as being in need of treatment for a microbial contamination, colonization, or infection. In general, a microbial colonization or infection may be induced by proliferation of a pathogenic microorganism (disease-causing microorganism). The microbial contamination may be identified by presence of one or more microorganism. In some embodiments, the methods may be employed for prevention or treatment of a microbial colonization or infection. The microbial colonization may refer to an established colony of one or more microorganism. The microbial infection may refer to an established colony of one or more microorganism which has been diagnosed by a clinical assessment. The microbial colonization or infection may be localized or systemic. In general, a microbial contamination may develop into a microbial colonization or infection if adequate treatment is not provided.

The preparation may be administered in an amount effective to treat biofilm or a microbial infection. The methods may generally comprise administering the preparation in an amount effective to promote deactivation of a pathogenic microorganism. In certain embodiments, the pathogenic microorganism may be a pathogenic bacterial organism. For example, the preparations and methods may be effective at promoting deactivation of broad-spectrum (gram-positive and gram-negative) bacteria. The pathogenic microorganism may be a species of a genus selected from Bacillus, Bartonella, Bordetella, Borrelia, Brucella, Campylobacter, Chlamydia, Chlamydophila, Clostridium, Corynebacterium, Enterococcus, Escherichia, Francisella, Haemophilus, Helicobacter, Legionella, Leptospira, Listeria, Mycobacterium, Mycoplasma, Neisseria, Pseudomonas, Rickettsia, Salmonella, Shigella, Staphylococcus, Streptococcus, Treponema, Ureaplasma, Vibrio, and Yersinia.

The preparation may be administered in combination with a surgical procedure. The methods may comprise administering the preparation in an amount effective to sterilize at least 90% of the target microorganism at the target site. For instance, the methods may comprise administering the preparation in an amount effective to sterilize at least 90%, at least 95%, at least 97%, at least 98%, at least 99%, at least 99.9%, at least 99.99%, or at least 99.999% of the target microorganism at the target site. Exemplary target sites include epithelial tissue, gastrointestinal system tissue, respiratory system tissue, cardiac system tissue, nervous system tissue, reproductive system tissue, ocular tissue, auditory tissue, and bloodstream. Epithelial tissue may include, for example, epidermis, dermis, hair, and nail. However, additional target sites may be treated by the methods disclosed herein. As disclosed herein, sterilize may refer to any process that eliminates, removes, kills, or deactivates the microorganism at the target site.

### Methods of Treatment of Fungal Infection

The preparation may be formulated to provide antifungal properties upon administration at a target site. For example, the self-assembled polymeric hydrogel may have antifungal properties. As disclosed herein, “antifungal” properties may refer to an effect against a fungal population, e.g., killing or inhibiting one or more organism from a fungal population. Thus, methods of treating a fungal infection or inhibiting proliferation of a fungal organism are disclosed herein. The methods may generally comprise administering the preparation in an amount effective to promote deactivation of a fungal organism. Methods of reducing or eliminating a fungal contamination are disclosed herein. In exemplary embodiments, a preparation comprising about 3.0% w/v or less of the peptide, for example, 1.5% w/v or less, or 1.0% w/v or less, may provide antifungal properties at a target site.

The methods may comprise identifying a subject as being in need of treatment for a fungal contamination, colonization, or infection. In certain embodiments, the preparation may be administered in an amount effective to treat at least one of biofilm, Tinea corporis, Candidiasis, Blastomycosis, Coccidioidomycosis, Histoplasmosis, Cryptococcosis, Paracoccidioidomycosis, Aspergillosis, Aspergilloma, Meningitis, Mucormycosis, Pneumocystis pneumonia (PCP), Talaromycosis, Sporotrichosis, and Eumycetoma of the subject. In some embodiment, the fungal organism may be a species of a genus selected from Aspergillus and Candida.

The preparations and methods may be effective at promoting deactivation of broad-spectrum (sporulating and non-sporulating) fungal organisms. The preparation may be administered in an amount effective to treat a fungal infection associated with or inhibit proliferation of at least one of Aspergillus clavatus, Aspergillus fischerianus, Aspergillus flavus, Aspergillus fumigatus, Aspergillus niger, Trichophyton mentagrophytes, Trichophyton rubrum, Microsporum canis, Candida albicans, Candida auris, Candida parapsilosis, Candida tropicalis, Blastomyces dermatitidis, Coccidioides immitis, Coccidioides posadasii, Cryptococcus gattii, Cryptococcus neoformans, Histoplasma capsulatum, Paracoccidioides brasiliensis, Pneumocystis jirovecii, Mucormycetes rhizopus, Mucormycetes mucor, Mucormycetes lichtheimia, Talaromyces marneffei, Sporothrix schenckii, Acremonium strictum, Noetestudina rosatii, Phaeoacremonium krajdenii, Pseudallescheria boydii, Curvularia lunata, Cladophilaophora bantiana, Exophiala jeanselmei, Leptosphaeria senegalensis, Leptosphaeria tompkinsii, Madurella grisea, Madurella mycetomatis, Pyrenochaeta romeroi, Trichosporon asahii, Cladosporium herbarum, and Fusarium sporotrichioides.

The preparation may be administered in combination with a surgical procedure. The methods may comprise administering the preparation in an amount effective to sterilize at least 90% of the fungal organism at the target site. For instance, the methods may comprise administering the preparation in an amount effective to sterilize at least 90%, at least 95%, at least 97%, at least 98%, at least 99%, at least 99.9%, at least 99.99%, or at least 99.999% of the fungal organism at the target site. Exemplary target sites may include epithelial tissue, oral tissue, esophageal tissue, tracheal tissue, pulmonary tissue, cardiac tissue, kidney tissue, ocular tissue, and bloodstream. Epithelial tissue may include, for example, epidermis, dermis, hair, and nail. However, additional target sites may be treated by the methods disclosed herein. As disclosed herein, sterilize may refer to any process that eliminates, removes, kills, or deactivates the fungal organism at the target site.

### Methods of Treatment of Biofilm

The preparation may be formulated for treatment of biofilm. Thus, the methods disclosed herein may comprise treatment of biofilm. Treatment of biofilm may generally comprise eliminating at least a portion of biofilm or inhibiting biofilm formation. Administration of the preparation may have an effect against a biofilm population, for example, killing or inhibiting one or more organism in a biofilm community. In general, the charged peptide polymer hydrogel may deconstruct the polymicrobial fungal and bacterial biofilm barrier upon contact. While not wishing to be bound by theory, it is believed the preparations disclosed herein may be selected to dismantle extracellular matrix of the biofilm population, exposing fungal, viral, and microbial organisms of the biofilm to the cationic peptide of the hydrogel. The peptide hydrogel may be effective by destroying microbes, fungi, and viral organisms within biofilms. The preparation may be administered as an antifungal, antimicrobial, and/or antiviral peptide to destroy fungi, microorganisms, and/or viral organisms, e.g., in a biofilm population, through cell lysis.

Methods of management of biofilm are also disclosed herein. For example, the methods may be employed for prevention of biofilm. The preparation may be administered to a target tissue having a population of biofilm or identified as prone to developing biofilm, e.g., a wound or wounded tissue. The preparation may be administered in response to tissue contamination or odor.

The methods may generally comprise administering the preparation in an amount effective to promote treatment of biofilm and/or deactivation of a biofilm population. The biofilm population may comprise bacterial organisms, for example, gram-positive and/or gram-negative bacterial organisms. The biofilm population may comprise fungal organisms, for example, sporulating and/or non-sporulating fungal organisms. Thus, the preparation may provide treatment of biofilm by the antimicrobial and/or antifungal properties and methods described above. In certain embodiments, the biofilm population may comprise viral organisms. The preparation may provide treatment of biofilm by antiviral properties and methods described herein.

The preparation may be formulated as a biofilm removal agent. In some embodiments, the preparation may be administered to a target tissue for removal of biofilm. For example, the preparation may be administered for debridement of the biofilm and/or biofilm-infected tissue.

### Methods of Treatment of Viral Infection

The preparation may be formulated to provide antiviral properties upon administration at a target site. For example, the self-assembled polymeric hydrogel may have antiviral properties. As disclosed herein, “antiviral” properties may refer to an effect against a viral population, e.g., killing or inhibiting one or more organism from a viral population. Thus, methods of treating a viral infection or inhibiting proliferation of a viral organism are disclosed herein. The methods may generally comprise administering the preparation in an amount effective to promote deactivation of a viral organism. Methods of reducing or eliminating a viral contamination are disclosed herein. In exemplary embodiments, a preparation comprising about 3.0% w/v or less of the peptide, for example, 1.5% w/v or less, or 1.0% w/v or less, may provide antiviral properties at a target site.

The methods may comprise identifying a subject as being in need of treatment for a viral contamination, colonization, or infection. In certain embodiments, the preparation may be administered in an amount effective to treat at least one of a respiratory viral colonization or infection (e.g., associated with rhinovirus, influenza, coronavirus, or respiratory syncytial virus), a viral skin infection (e.g., associated with molluscum contagiosum, herpes simplex virus, or varicella-zoster virus), a foodborne viral infection (e.g., associated with hepatitis A, norovirus, or rotavirus), a sexually transmitted viral infection (e.g., associated with human papilloma virus, hepatitis B, genital herpes, or human immunodeficiency virus), and other viral infections (e.g., associated with Epstein-Barr virus, West Nile virus, or viral meningitis) of the subject.

The preparation may be administered in combination with a surgical procedure. The methods may comprise administering the preparation in an amount effective to sterilize at least 90% of the viral organism at the target site. For instance, the methods may comprise administering the preparation in an amount effective to sterilize at least 90%, at least 95%, at least 97%, at least 98%, at least 99%, at least 99.9%, at least 99.99%, or at least 99.999% of the viral organism at the target site or systemically. In some embodiments, the methods may comprise administering the preparation in an amount effective to sterilize 100% of the viral organism at the target site or systemically. In certain embodiments, the preparation may be administered in an amount effective to treat biofilm or kill or deactivate a biofilm population containing a viral organism.

Exemplary target sites may include epithelial tissue, oral tissue, esophageal tissue, tracheal tissue, pulmonary tissue, cardiac tissue, kidney tissue, ocular tissue, and bloodstream. However, additional target sites may be treated by the methods disclosed herein. As disclosed herein, sterilize may refer to any process that eliminates, removes, kills, or deactivates the viral organism at the target site.

### Methods of Administration of Peptide Hydrogels

The peptide hydrogels may be administered by any mode of administration known to one of skill in the art. The method of administration may comprise selecting a target site of a subject and administering the preparation to the target site. In certain embodiments, the methods may comprise mixing the peptide with a buffer configured to induce self-assembly of the peptide to form the hydrogel. In general, the peptide may be mixed with the buffer prior to administration. However, in some embodiments, the peptide may be combined with the buffer at the target site.

The target site may be any bodily tissue or bloodstream. In some embodiments, the target site may be epithelial tissue, gastrointestinal system tissue, respiratory system tissue, cardiac system tissue, nervous system tissue, reproductive system tissue, ocular tissue, or auditory tissue. The route of administration may be selected based on the target tissue. Exemplary routes of administration are discussed in more detail below.

In some embodiments, the methods may comprise identifying a subject in need of administration of the preparation. The methods may comprise imaging a target site or monitoring at least one parameter of the subject, systemically or at the target site. Exemplary parameters which may be monitored include temperature, pH, response to optical stimulation, and response to dielectric stimulation. Thus, in some embodiments, the method may comprise providing optical stimulation or dielectric stimulation to the subject, optionally at the target site, for measuring a response. The response may be recorded, optionally in a memory storing device. In general, any parameter which may inform a user of a need or desire for administration of the preparation may be monitored and/or recorded. The methods may comprise imaging the target site or monitoring at least one parameter of the subject prior to administration of the preparation, concurrently with administration of the preparation, or subsequent to administration of the preparation. For example, the methods may comprise imaging the target site or monitoring at least one parameter of the subject after an initial dose and before a potential subsequent dose of the preparation.

In certain embodiments, the preparation may be administered responsive to the measured parameter being outside tolerance of a target value. The preparation may be administered automatically or manually in response to the measured parameter.

The preparation may be formulated for topical, parenteral, or enteral administration. The preparation may be formulated for systemic administration. Various pharmaceutically acceptable carriers and their formulations are described in standard formulation treatises, e.g., Remington's Pharmaceutical Sciences by E. W. Martin. See also Wang, Y. J. and Hanson, M. A., Journal of Parenteral Science and Technology, Technical Report No. 10, Supp. 42:2 S, 1988; Aulton, M. and Taylor, K., Aulton's Pharmaceutics: The Design and Manufacture of Medicines, 5th Edition, 2017; Antoine, A., Gupta M. R., and Stagner, W. C., Integrated Pharmaceutics: Applied Preformulation, Product Design, and Regulatory Science, 2013; Dodou K. Exploring the Unconventional Routes-Rectal and Vaginal Dosage Formulations, The Pharmaceutical Journal, 29 Aug. 2012.

### Parenteral Administration of Peptide Hydrogels

In certain embodiments, the hydrogels may be administered parenterally. In general, parenteral administration may include any route of administration that is not enteral. The preparation may be administered parenterally via a minimally invasive procedure. In particular embodiments, the parenteral administration may include delivery by syringe, e.g., by needle, or catheter. For instance, the parenteral administration may include delivery by injection. The parenteral administration may be intramuscular, subcutaneous, intravenous, or intradermal. The shear-thinning ability of the hydrogels may allow distribution through small lumens, while still providing minimally invasive treatment.

The method may comprise applying mechanical force to the hydrogel for parenteral administration. The hydrogel may be thinned by applied mechanical force, for example, pressure applied by a syringe to administer the preparation by injection. In particular, the pressure applied to administer the preparation through a needle or catheter may be sufficient to shear thin the hydrogel for application.

The peptide hydrogels may be administered parenterally to any internal target site in need thereof. For instance, cardiac tissue, nervous tissue, connective tissue, epithelial tissue, or muscular tissue, and others, may be the target site. The peptide hydrogels may be administered parenterally to a target site of a solid tumor. In exemplary embodiments, antifungal treatment of pulmonary tissue may be provided by parenteral administration of the peptide hydrogels described herein.

### Topical Administration of Peptide Hydrogels

In certain embodiments, the hydrogels may be administered topically. In general, topical administration may include any external or transdermal administration. For instance, the target site for administration may be an epithelial tissue. In particular embodiments, the topical administration may be accompanied by a wound dressing or hemostat.

The preparation may be administered topically with a delivery device. For instance, the preparation may be administered topically by spray, aerosol, dropper, tube, ampule, film, or syringe. In particular embodiments, the preparation may be administered topically by spray. The spray may be, for example, a nasal spray. Spray parameters which may be selected for administration include droplet size, spray pattern, capacity, spray impact, spray angle, and spray diameter. Thus, the methods may comprise selecting a spray parameter to correlate with the target site or target indication. For instance, a smaller spray diameter may be selected for administration to a small wound. A specific spray angle may be selected for administration to a target site which is difficult to reach. A denser spray pattern or larger droplet size may be selected for administration to a moist target site.

Exemplary droplet sizes may be between 65 μm to 650 μm. For instance, fine droplets having an average diameter of 65 μm to 225 μm, medium droplets having an average diameter of 225 μm to 400 μm, or coarse droplets having an average diameter of 400 μm to 650 μm may be selected. The spray pattern may range from densely packed droplets to sparse droplets. The spray diameter may range from less than 1 cm to 100 cm. For instance, spray diameter may be selected to be between less than 1 cm and 10 cm, between 10 cm and 50 cm, or between 50 cm and 100 cm. Spray angle may range from 0° to 90°. For instance, spray angle may be selected to be between 0° and 10°, between 10° and 45°, or between 45° and 90°.

In some embodiments, the preparation may be administered topically with a film. The film may be a rigid, semi-flexible, or flexible film. In certain embodiments, the flexible or semi-flexible film may be configured to adopt a topological conformation of the target site. In general, the film may be in the form of a substrate saturated with the preparation or hydrogel. The substrate may be rigid, semi-flexible, or flexible. The film may be administered as a barrier dressing and/or hemostat. The preparation may be administered topically with a film and accompanied by a barrier dressing.

The peptide formulated as a saturated film or barrier dressing may provide antimicrobial, antiviral, and/or antifungal treatment by direct contact with target population, as previously described. Conventional antimicrobial wound dressings rely on traditional antibiotics and function merely as a vehicle for antibiotic agents. However, the peptide hydrogel saturated film or barrier dressing described herein may be designed to provide a biophysical mode of cell-membrane disruption against broad-spectrum (gram-positive and gram-negative) bacterial cultures. Thus, the antimicrobial, antiviral, and/or antifungal peptide hydrogel saturated film or barrier dressing may generally avoid concerns around minimum inhibitory bacterial concentrations typical to conventional small molecule loaded polymers. Instead, the peptide hydrogel disclosed herein may be designed to exert toxicity against gram-positive and gram-negative bacteria (including antibiotic resistant strains) while remaining cell-friendly, non-inflammatory, and non-toxic by selecting amino acid charge ratio of the peptide. Similarly, the peptide hydrogel disclosed herein may be designed to exert toxicity against fungal organisms (e.g., sporulating and non-sporulating fungal organisms) and/or viral organisms. The saturated film or barrier dressing disclosed herein may provide a temporary extracellular matrix scaffold for tissue regeneration.

The peptide hydrogels may be administered topically to any target site in need thereof. Wound healing, e.g., diabetic wound healing, is described herein as one exemplary embodiment. However, it should be understood that many other topical target sites and treatments are envisioned, for example, as previously described above. The wounds may include acute, sub-acute, and chronic wounds. The wound may be a surgical wound or ischemic wound. Chronic wounds such as venous and arterial ulcer wounds or pressure ulcer wounds, and acute wounds, such as those caused by trauma may be treated. In some embodiments, the preparation may be formulated as a film, barrier dressing, and/or hemostat. Administration of the preparation may accompany a barrier dressing and/or hemostat.

Treatment and/or management or inhibition of biofilm is described herein as another exemplary embodiment. Tissue hydration is described herein as another exemplary embodiment. Moisture management and/or exudate management of wounds or tissues is described herein as another exemplary embodiment. Tissue debridement is described herein as another exemplary embodiment. The preparation may be administered topically as a prophylactic, for example, in association with a wound. The preparation may be administered topically as an analgesic, for example, to a chronic wound or site of biofilm.

### Enteral Administration of Peptide Hydrogels

In certain embodiments, the hydrogels may be administered enterally. In general, enteral administration may include any oral or gastrointestinal administration. For instance, the target site for administration may be an oral tissue or a gastrointestinal tissue. In particular embodiments, the enteral administration may be accompanied by food or drink. The preparation may be administered on a substantially empty stomach. In some embodiments, water is administered to the subject after administration of the preparation. In some embodiments, several hours are waited prior to food consumption after administration.

Such enteral preparations may be formulated for oral, sublingual, sublabial, buccal, or rectal application. Oral application formulations are generally prepared specifically for ingestion through the mouth. Sublingual and sublabial formulations, e.g., tablets, strips, drops, sprays, aerosols, mists, lozenges, and effervescent tablets, may be administered orally for diffusion through the connective tissues under the tongue or lip. Specifically, formulations for sublingual administration may be placed under the tongue and formulations for sublabial administration may be placed between the lip and gingiva (gum). Sublabial administration may be beneficial when the dosage form comprises materials that may be corrosive to the sensitive tissues under the tongue. Buccal formulations may generally be topically held or applied in the buccal area to diffuse through oral mucosa tissues that line the cheek. Rectal application may be achieved by inserting the formulation in the rectal cavity, either with or without the assistance of a device. Device-assisted application may include, for example, delivery via an applicator or an insertable applicator, catheter, feeding tube, or delivery in conjunction with an endoscope or ultrasound. Suitable applicators include liquid formulation bulbs and launchers and solid formulation insertable applicators.

For any of the routes of administration disclosed herein, the methods may comprise administering a single dosage of the preparation. The site of administration may be monitored for a period of time to determine whether a booster or subsequent dosage of the preparation is to be administered. For example, the methods may comprise monitoring the site of administration. A parameter of the subject, optionally at the target site, may be monitored as previously described. The subject may be monitored hourly, every 2-3 hours, every 6-8 hours, every 10-12 hours, every 12-18 hours, or once daily. The subject may be monitored daily, every other day, once every few days, or weekly. The subject may be monitored monthly or bi-monthly. In certain embodiments, the subject may be monitored for a period of up to about 6 months. For example, the subject may be monitored for about 1 month, about 2 months, about 3 months, about 4 months, about 5 months, or about 6 months.

The methods may comprise administering at least one booster or subsequent dosage of the preparation. For example, the methods may comprise administering a booster dosage to the target site at least about 1 day, 2 days, 3 days, 4 days, 5 days, 6 days, 7 days, 8 days, 9 days, 10 days, 11 days, 12 days, 13 days, or 14 days after the first dosage. The methods may comprise administering a booster dosage 1 week, 2 weeks, 3 weeks, 4 weeks, 5 weeks, or 6 weeks after the first dosage. The methods may comprise administering a booster dosage at least 6 months or 1 year after the first dosage. In certain embodiments, at least a portion of the hydrogel may be present at the target site at the time of the booster dosage. In other embodiments, the hydrogel may be fully metabolized or otherwise eliminated from the target site at the time of the booster dosage.

Methods of Biological Material Delivery with Peptide Hydrogels

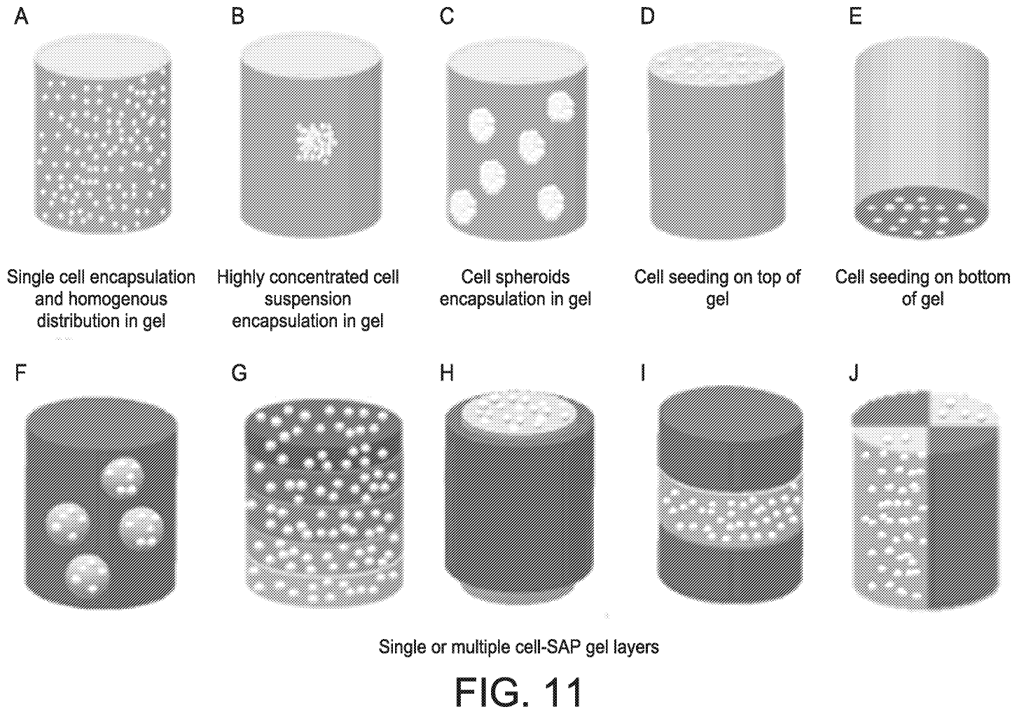

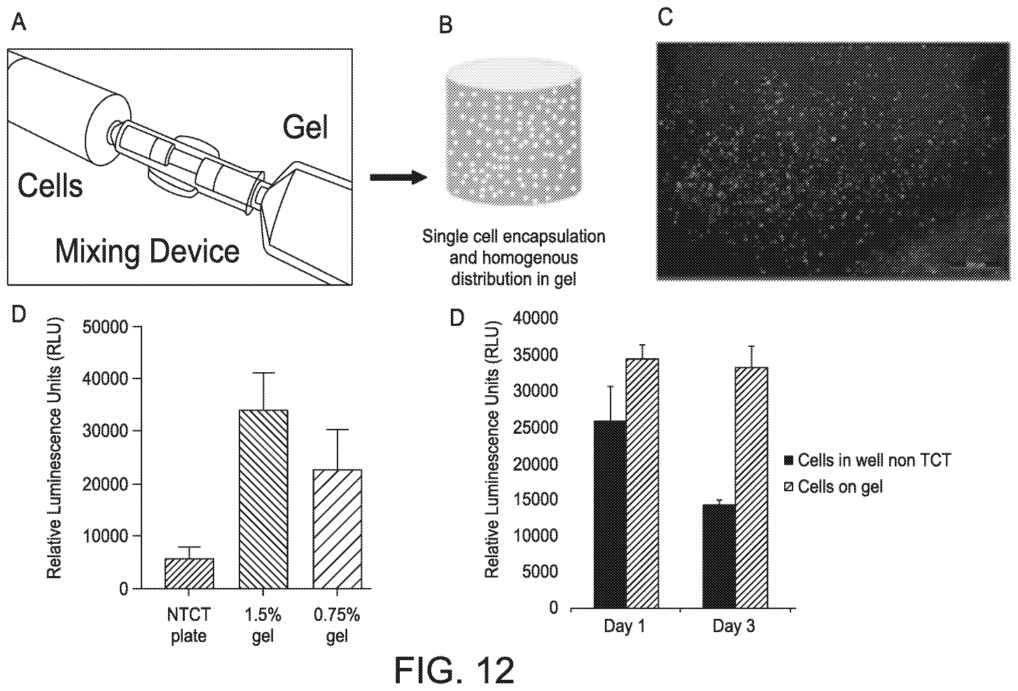

Methods of administering biological material to a subject are disclosed herein. The methods may generally include suspending the biological material in a hydrogel. The biological material may be combined with a preparation comprising a self-assembling peptide in a biocompatible solution and a buffer comprising an effective amount of an ionic salt and a biological buffering agent to induce self-assembly of the hydrogel. The methods may comprise administering an effective amount of the biological material, the preparation, and the buffer (optionally in hydrogel form) to a target site of the subject. Suspending the biological material with the preparation or the buffer will generally produce a liquid suspension. Combining the preparation with the buffer may trigger gelation of the suspension into a hydrogel comprising the biological material, for example, as shown in FIGS. 11 and 12.

In certain embodiments, the biological material may be suspended in the preparation before inducing gelation. In other embodiments, the preparation comprising the self-assembling peptide in biocompatible solution may be combined with the buffer prior to suspending the cells in the hydrogel. In such embodiments, the biological material may be seeded on the hydrogel or encapsulated within the hydrogel to form a non-homogeneous suspension.

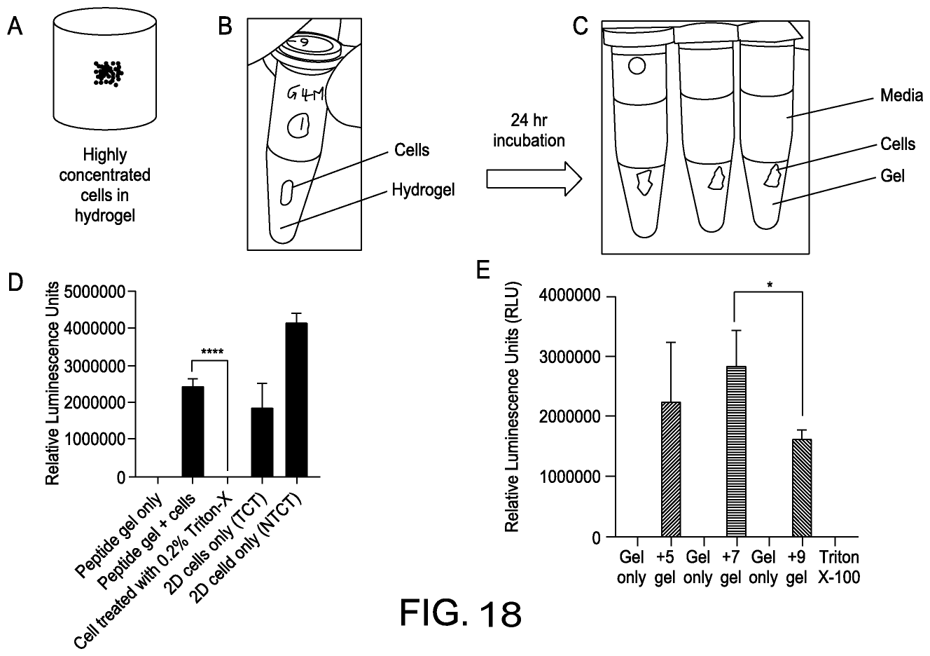

The non-homogeneous suspension may include clusters or spheroids of biological material (FIG. 11). The clusters or spheroids generally refer to highly concentrated biological material aggregates within the hydrogel. In certain embodiments, hydrogels comprising clusters or spheroids of biological material may exhibit greater viability, growth, or function of the biological material after administration to the target tissue. As shown in the data presented in FIG. 18, murine MSC cluster in pre-formed hydrogels (n=3) showed good cell viability in self-assembling peptide matrices, as measured by relative luminescence units (RLU) using a standard luciferase viability assay at 24 hours post-incubation. Thus, it is believed clusters and spheroids may provide increased viability of biological material by reducing the effect from charge of the hydrogel.

The buffer may generally comprise an effective amount of an ionic salt and a biological buffering agent to control one or more properties of the formed hydrogel, such as stiffness, pH, and water content. The composition of the buffer may be selected to trigger a degree of gelation, a desired pH, or a desired water content. The properties of the formed hydrogel may be selected based on a biological material type or target tissue.

Methods of treating tissue injury are disclosed herein. The preparation and/or buffer may be administered in an effective amount of the biological material to treat tissue injury, e.g., internal tissue injury. Thus, the methods disclosed herein may comprise administering the biological material, the preparation, and the buffer to internal tissue as the target tissue. The tissue injury treated may be acute, sub-acute, or chronic. The methods may comprise identifying a subject as being in need of treatment for tissue injury. The methods may comprise identifying a target tissue as being in need of treatment.

The preparations disclosed herein may be administered in combination with a treatment or agent to enhance cell or tissue graft therapy. In certain embodiments, the preparations disclosed herein may be administered in combination with a treatment or agent to prevent or inhibit cell death, to control or reduce inflammation, and/or to promote nerve regeneration. In certain embodiments, the preparations disclosed herein may be administered in combination with a treatment to enhance or inhibit tissue protein expression such as to increase or decrease melanosome production, collagen and/or elastin fiber production, or histamine production.

Methods of in situ tissue regeneration are disclosed herein. The methods may comprise administering biological material in an amount effective for tissue regeneration. The hydrogel may generally provide a biocompatible biological material delivery system configured to localize the biological material at the site of tissue regeneration and improve biological material retention. The biological material may comprise cells, for example, multipotent stem cells, pluripotent stem cells, bone marrow mononuclear cells, or cells of the target tissue. The biological material may comprise tissue material or tissue-derived material, for example, of the target tissue. The biological material may comprise biological fluids, for example, natural biological fluids or synthetic biological fluids, for example, having a composition effective to provide stem cell differentiation into cells of the target tissue.

The hydrogels disclosed herein may be employed as biological material delivery systems for the regeneration of many types of tissues including, but not limited to, nerve tissue, bone tissue, and connective tissue. The hydrogels may be designed to incorporate essential mechanical properties, cell-adhesion properties, or biochemical properties for each target tissue and indication. By designing the self-assembling peptide sequence, adding bioactive motifs, or altering the gel formulation, the hydrogels may be tailored for the specific needs of the selected biological material, such that tissue regeneration outcomes can be improved.

In one exemplary embodiment, the methods of tissue regeneration may comprise providing biological material therapy for transplanted tissues. The methods may comprise administering the biological material, peptide preparation, and buffer as disclosed herein in combination with a tissue transplantation procedure. The methods may be implemented with neural tissue transplantation, connective tissue transplantation, bone transplantation, or other types of transplantation. As previously described, the biological material and peptide may be selected and/or designed based on the target tissue and indication to be treated. Particular biological materials may be selected in combination with selected transplantation procedures.

The preparation and/or buffer may be administered directly to the site for treatment, e.g., topically or parenterally for treatment of tissue injury.

The methods may comprise administering the suspension and/or buffer to a target tissue selected from mesenchymal tissue, connective tissue, muscle tissue, nervous tissue, embryonic tissue, dermal tissue, bone tissue, dental tissue, corneal tissue, cutaneous tissue, integumental tissue, soft tissue, and hard tissue. In some embodiments the connective tissue may comprise cartilage. The methods may comprise administering the suspension and/or buffer to a biological fluid selected from tears, mucus, urine, menses, blood, wound exudates, and mixtures thereof.

The methods may comprise administering a first dosage of the biological material, preparation, and/or buffer. The methods may comprise administering at least one booster dosage of the biological material, preparation, and/or buffer. The methods may comprise monitoring the site of administration to determine whether to administer the at least one booster dosage, for example, responsive to one or more indication. In certain embodiments, the methods may comprise administering the at least one booster dosage after a predetermined period of time, for example, after 1 day, 1 week, 2 weeks, 1 month, 2 months, 3 months, 6 months, or 12 months.

The methods may be performed in vitro or in vivo. In certain embodiments, the biological material suspension may be administered to the target site before administration of the buffer. In such embodiments, the hydrogel may be formed in vivo. In other embodiments, the biological material suspension may be combined with the buffer prior to administration of either component. In such embodiments, the hydrogel may be formed in vitro.

In accordance with certain embodiments, the methods may be performed in situ. In such embodiments, the biological material may be administered to the target site and the self-assembling peptide and buffer may be administered to the target site independently. The biological material may be administered to the target site before the self-assembling peptide and the buffer. The self-assembling peptide and the buffer may be administered to the target site before the biological material. The self-assembling peptide and the buffer may be administered independently or combined, as a hydrogel.

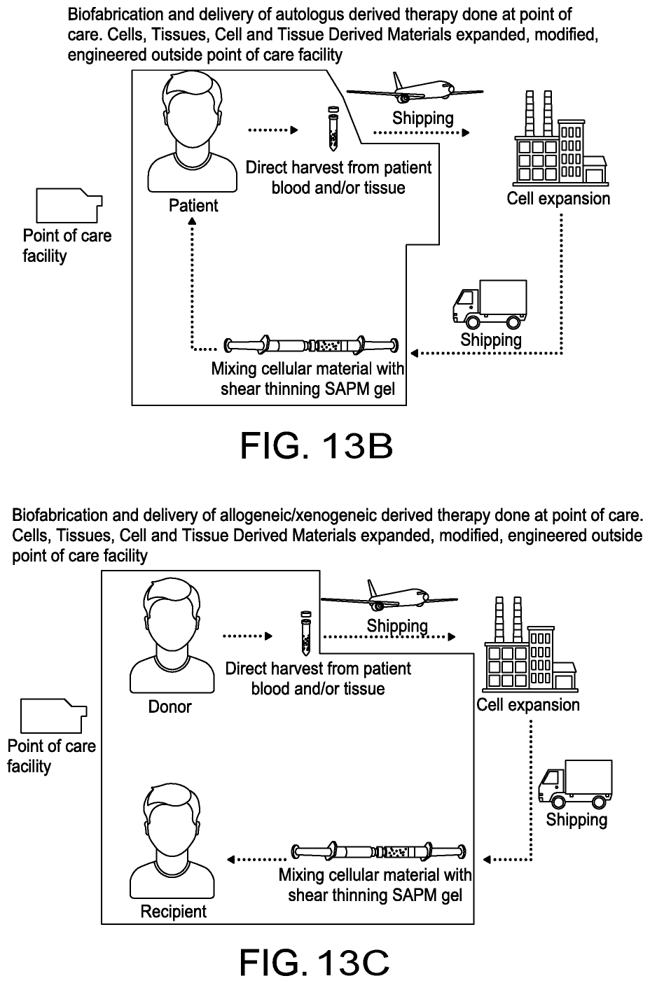

As shown in FIG. 14, cells may be administered to the target site after administration of the self-assembling peptide and buffer. Image (A) is a schematic diagram of exemplary cells seeded on top of the hydrogel. The photograph of image (B) shows murine mesenchymal stem cells (MSCs) seeded on top of the hydrogel. The photograph of image (C) shows the seeded cells are readily absorbed onto the hydrogel without any loss of cells during delivery. Image (D) is a bright-field (BF) microscopy image of murine MSCs spreading well on top of the hydrogel. The data presented in graphs (E) and (F) shows MSCs seeded on top of self-assembling peptide (SAP) hydrogel (n=4) show greater cell viability than MSCs seeded on 2D culture and on competitor hydrogels, including competitor products containing collagen, as measured by relative luminescence units (RLU) using a standard luciferase viability assay

In accordance with certain embodiments, methods of culturing cells in vitro are provided. The methods may generally include suspending the cells in the preparation comprising the self-assembling peptide and combining the cell suspension with the buffer to induce gelation of the self-assembling peptide and produce the cell culture.

In accordance with certain embodiments, methods of preparing a tissue graft in vitro are provided. The methods may generally include suspending the tissue material in the preparation comprising the self-assembling peptide and combining the tissue suspension with the buffer to induce gelation of the self-assembling peptide and produce the tissue graft.

In accordance with certain embodiments, methods of preparing a cell culture or tissue graft in situ are provided. The methods may generally include administering the preparation comprising the peptide in a biocompatible solution to the target site. The methods may include administering the buffer configured to induce self-assembly of the peptide to the target site. The methods may comprise administering the biological material to the hydrogel in situ to localize the administered biological material.

The methods may comprise culturing or grafting the biological material in the preparation for a predetermined period of time prior to administration. The methods may comprise combining one or more of a cell culture media, a cell maintenance agent, a cell growth agent, and a cell culture serum with the cell culture or tissue graft. In some embodiments, the methods may comprise culturing or grafting the biological material in the hydrogel, for example, by combining the preparation with the buffer, for a predetermined period of time prior to administration. The period of time may be any period of time sufficient to grow or seed the biological material to a desired degree. Thus, the period of time may generally depend on the biological material type, the culture or graft conditions, and the desired outcome of the culture or graft prior to administration. The period of time may be, for example, at least 1 hour, 2 hours, 3 hours, 6 hours, 12 hours, 18 hours, 24 hours, 2 days, 3 days, 4 days, 5 days, 6 days, 1 week, 2 weeks, 3 weeks, or 1 month.

The biological material to be administered may include biological fluids, cells, and/or tissue material. In some embodiments, one or more biological material administered may be synthetic. For instance, the biological fluids may be or include synthetic fluids. In other embodiments, the biological material may be obtained from a donor. The biological material may be autologous (obtained from the recipient subject). The biological material may be allogeneic (obtained from a donor subject of the same species as the recipient subject) or xenogeneic (obtained from a donor subject of a different species as the recipient subject).

The cells to be administered may include whole cells, live cells, dead cells, cell-derived matter, and/or cell fragments or cell lysates. The cells may include at least one of eukaryote cells, virus, prokaryote cells, adjuvants, cytokines, and growth factors. The cells may include, for example, progenitor cells, multipotent cells, induced pluripotent cells, immune cells, specialized cells, terminally differentiated cells, bone marrow mononuclear cells, islet cells, and combinations thereof.

The cell-derived matter may include secreted material, cell fragments, and other byproducts produced by cells, by cell activity, or from cells. For example, cell-derived matter may comprise secretomes, exosomes, proteins (e.g., signaling proteins), enzymes, biological fluids, serum, plasma, spheroids, liposomes, growth factors, cytokines, chemokines, paracrine factors, autocrine factors, genetic material (e.g., RNA or DNA), vesicles, and/or granules.

The cells may be or include mesenchymal stem/stromal cells (MSCs). Mesenchymal stem/stromal cells (MSCs) are multipotent cells. MSCs may be isolated from multiple tissues, including bone marrow and adipose tissue. Therapeutic treatment with MSCs may be employed for a variety of disease indications. MSCs typically provide therapeutic effects by, for example, secreting bioactive factors that act in a paracrine manner to recruit other cell types and enhance angiogenesis and tissue regeneration. Such therapeutic effects of MSCs may be generally employed in wound healing, e.g., chronic wound healing.

The cells may be or include stem cells. The stem cells may be pluripotent. The stem cells may be non-pluripotent. In some embodiments, the stem cells may be differentiated. For example, the stem cells may be differentiated into a target cell type. In other embodiments, the stem cells may be non-differentiated. The stem cells may be terminally-differentiated cells, de-differentiated cells, specialized cells, or combinations thereof. Exemplary stem cells include neural stem cells, adipose-derived stem cells, bone marrow mesenchymal stem cells, muscle-derived stem cells, hair follicle stem cells, dental pulp stem cells, skin-derived stem cells, or induced pluripotent stem cells. In certain exemplary embodiments, the stem cells may include bone marrow derived stromal cells and/or adipose derived stromal cells. The stem cells may be embryonic stem cells and/or adult stem cells.

The cells may be or include glial cells. Glial cells are non-neuronal cells found in the central nervous system (CNS) and peripheral nervous system (PNS). Glial cells function to support neurons. Glial cells may provide physical support for neurons, be a source of nutrients to neurons, regulate extracellular fluid of the nervous system (for example, surrounding neurons and synapses), direct the migration of neurons, and produce molecules that modify the growth of axons and dendrites. Glial cells administered by the methods disclosed herein may have an effect on regeneration of lost neural functioning.

The cells may be glial cells, including one or more of oligodendrocytes, astrocytes, ependymal cells, microglia, Schwann cells, and satellite cells. Oligodendrocytes form the myelin sheath in the CNS. Astrocytes have an effect on regulating the external chemical environment of neurons. Ependymal cells create and secrete cerebrospinal fluid (CSF). Under certain conditions, ependymal cells may act as neural stem cells. Microglia exhibit phagocytotic activity which protects neurons of the CNS. Schwann cells provide myelination to axons in the PNS. Under certain conditions, Schwann cells may exhibit phagocytotic activity which allows for regrowth of neurons. Satellite cells also have an effect on regulating the external chemical environment of neurons.

The cells may be or comprise Schwann Cells (SC). Schwann cells (SC), also called neurilemma cells, refer to the principal glia of the PNS. The Schwann cells may include myelinating and non-myelinating cells. Schwann cells may refer to any of the cells in the PNS that produce the myelin sheath around neuronal axons. Myelinating Schwann cells wrap around axons of motor and sensory neurons to form the myelin sheath. Schwann cells typically play a role in the conduction of nervous impulses along axons, nerve development and regeneration, trophic support for neurons, production of the nerve extracellular matrix, modulation of neuromuscular synaptic activity, and presentation of antigens to T-lymphocytes. Schwann cells may promote axon regeneration, axon re-innervation, and/or axon attachment. In some embodiments, the Schwann cells may be engineered to express one or more moiety of interest.

Schwann cells may include, for example, those cells disclosed in and/or prepared by the methods disclosed in U.S. Patent Application Publication No. 20190055515, titled “Production of Schwann Cells,” incorporated by reference herein in its entirety for all purposes.

The tissue to be administered may include live tissue, dead tissue, tissue-derived matter, and/or tissue fragments or tissue lysates. The tissue or tissue material may comprise, for example, bone tissue, connective tissue, neural tissue, adipose tissue, bone marrow, or combinations thereof. The biological material may comprise tissue or tissue material selected from mesenchymal tissue, connective tissue, muscle tissue, nervous tissue, embryonic tissue, dermal tissue, bone tissue, dental tissue, corneal tissue, soft tissue, and hard tissue. The tissue or tissue material may comprise a biological fluid selected from tears, mucus, urine, menses, blood, wound exudates, and mixtures thereof.

The self-assembled hydrogel may have a physical structure substantially similar to the native extracellular matrix of the biological material, e.g., target cell type, allowing the gel to serve as a temporary scaffold to promote growth, function, and/or viability. In particular, the self-assembled hydrogel may have similar properties, including, for example, pore size, density, hydration, charge, rigidity, etc., to the native extracellular matrix. The properties may be selected responsive to a target cell type.

The self-assembled hydrogel may have a selected degradation rate. The degradation rate may be selected responsive to the target site of implantation or administration. The self-assembled hydrogel properties may be selected to promote migration of the biological material, e.g., cells, to the hydrogel environment. The self-assembled hydrogel properties may be selected to promote protection in a hostile environment. The self-assembled hydrogel properties may be selected to promote anchoring of biological material within the hydrogel, for example, as with cells that will not engraft onto host tissue. The self-assembled hydrogel properties may be selected to promote migration of cell products or byproducts to the hydrogel environment, for example. In an exemplary embodiment, the self-assembled hydrogel properties may be selected to control differentiation of cells, e.g., progenitor cells or stem cells, e.g., mesenchymal stem cells.

The preparation and/or buffer may be sterile, e.g., substantially sterile. The preparation and/or buffer may be sterilized by terminal sterilization. The preparation and/or buffer may be sterilized by autoclave sterilization. The preparation and/or buffer may be substantially free of a preservative.

The hydrogels disclosed herein have gelation kinetics which are fast enough to ensure cells and tissue material becomes substantially homogeneously incorporated within the matrix. In particular, the gelation kinetics are sufficiently fast to afford an even distribution of encapsulated cells to allow reproducible control over cell density within the matrix. In other embodiments, biofabrication may comprise controlling the gelation kinetics to form a non-homogeneous suspension, e.g., suspended cell clusters or spheroids.

Additionally, the hydrogels disclosed herein have a construct that can be introduced in vivo and remain localized at the point of administration, for example, even without a spatial cavity. The localization of the hydrogel upon administration can limit or inhibit leakage of the cell construct to neighboring tissue.

The suspension and/or peptide may be engineered to express a desired property. In certain embodiments, the suspension and/or peptide may be engineered to protect cells from hostile environments. In particular, the suspension and/or peptide may be engineered to protect the cells from environments with a high microbial burden, hostile immune cells, or environmental proteins. The suspension and/or peptide may be engineered to increase cell viability. The suspension and/or peptide may be engineered to control differentiation, control cell fate in situ, control cell fate in vivo, control cell fate ex vivo, or control cell fate in vitro. The suspension and/or peptide may be engineered to increase cell attachment to the matrix or increase cell attachment and/or migration in the environment. The suspension and/or peptide may be engineered to decrease apoptosis, for example, by providing cell attachment and/or biological modulation.

The suspension and/or peptide may be engineered to achieve the results described above by altering the expression of protein motifs or the net charge of the peptides. The hydrogel properties may be engineered by controlling one or more of the expression of extracellular matrix or protein motifs, the presence or absence of fusion proteins, the net charge of the peptides, the presence or absence of cationic particles or peptides, the presence or absence of anionic particles or peptides, buffers, salts, peptide concentration, peptide purity, the presence or absence of peptide counterions, the presence or absence of specialized proteins, and the presence or absence of specialized small or large molecules.

The properties of the self-assembled hydrogel may be controlled by designing the peptide. For example, the peptide may include functional groups that provide one or more selected physical property. The properties may be controlled by selecting the composition of media or buffer. For example, the media may include serum or be substantially free of serum. For example, the buffer may have a net positive charge, be net neutral, or have a net negative charge. In some embodiments, the functional group may be configured to alter peptide net charge or counterions when the peptide is suspended in the solution.

The self-assembled hydrogel may be designed to have cell and/or tissue protective properties. In particular, the self-assembled hydrogel may be designed to be protective against foreign microorganisms, e.g., pathogenic microorganisms, as previously described. The self-assembled hydrogel may be designed to be protective against immune attack from environmental immune cells, for example, by providing a physical barrier or biochemical modulation. The antimicrobial and/or protective properties of the hydrogel may not substantially affect the viability, growth, or function of the engrafted biological material.

The protective properties of the hydrogel may be engineered by altering the net charge of the peptides. In some embodiments, the net charge may be altered by controlling one or more of the presence or absence of cationic particles or peptides, the presence or absence of anionic particles or peptides, buffers, salts, peptide concentration, peptide purity, and the presence or absence of peptide counterions.

The suspension may be designed to have a substantially physiological pH level. The suspension may have a pH level of between about 4.0 and 9.0. In some embodiments, the suspension may have a pH level of between about 6.0 and 9.0, for example between about 7.0 and 8.0. The suspension may have a pH level of between about 7.3 and 7.5, for example, about 7.4. The substantially physiological pH may allow administration of the biological material directly to a target tissue and/or at the time of preparation.

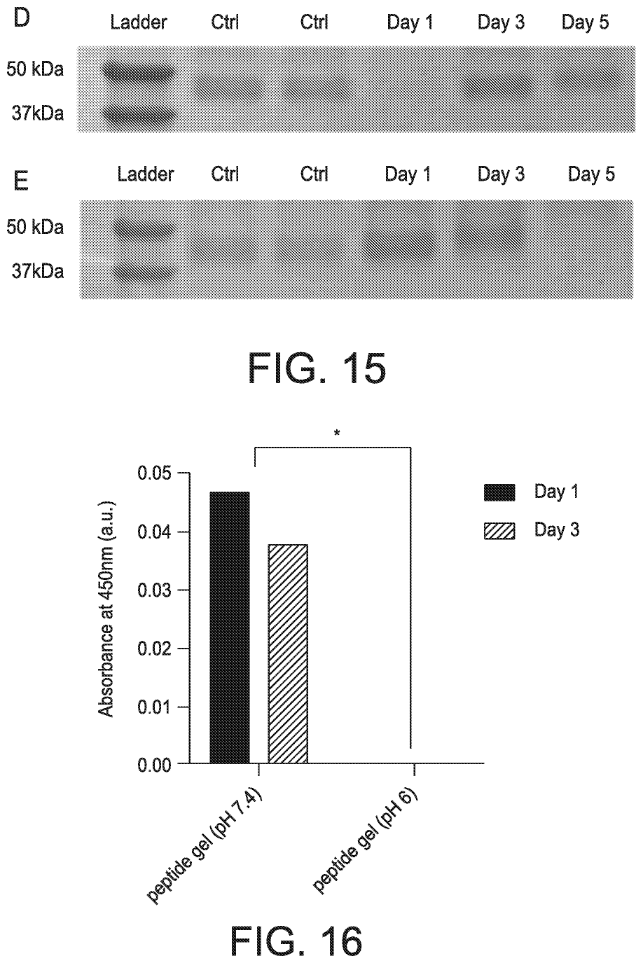

In one exemplary embodiment, murine mesenchymal stem cells (MSCs) were encapsulated into peptide hydrogels at physiological pH (about 7.4) and at pH 6 (FIG. 16). The MSCs encapsulated in hydrogel at physiological pH for 1 and 3 days showed greater cell viability than the peptide hydrogels at pH 6, as measured by absorbance at 450 nm using a standard absorbance viability assay. (p<0.05). The pH value of the hydrogel may be selected based on the selected biological material. Different biological material may show improved viability with encapsulation in different pH value hydrogels.

In some embodiments, the suspension may be prepared at a point of care. In some embodiments, the hydrogel may be prepared by combining the suspension with the buffer at the point of care. For example, the suspension and/or the buffer may be prepared less than about 1 minute, less than about 2 minutes, less than about 5 minutes, or less than about 10 minutes prior to administration. In autologous administration embodiments, the methods may comprise collecting or harvesting the biological material from the subject at the point of care, prior to preparing the suspension. The material may be collected or harvested from the subject less than about 1 minute, less than about 2 minutes, less than about 5 minutes, or less than about 10 minutes prior to suspending the biological material.

The methods may comprise suspending the biological material in the preparation, optionally, agitating the suspension to provide a substantially homogeneous or non-homogeneous distribution of the biological material, and administering the suspension at a point of care. In some embodiments, the suspension may be agitated to provide a substantially homogeneous distribution of the biological material. In other embodiments, the suspension may be prepared or agitated to provide a non-homogeneous suspension, for example, comprising clusters or spheroids of the biological material.

The administration of the suspension may be topical or parenteral, as described in more detail below.

The administration of cells and cell products or byproducts at the target site may be controlled by altering the release properties of the hydrogel. In some embodiments, the release properties may be engineered by controlling one or more of the expression of extracellular matrix or protein motifs, the presence or absence of fusion proteins, the net charge of the peptides, the presence or absence of cationic particles or peptides, the presence or absence of anionic particles or peptides, buffers, salts, peptide concentration, peptide purity, and the presence or absence of peptide counterions. The properties may be engineered to deploy cells or tissue at the target site. The properties may be engineered to deploy cell products or byproducts at the target site, for example, delivery of exosomes, growth factors, genetic material, RNA, siRNA, shRNA, miRNA, etc.

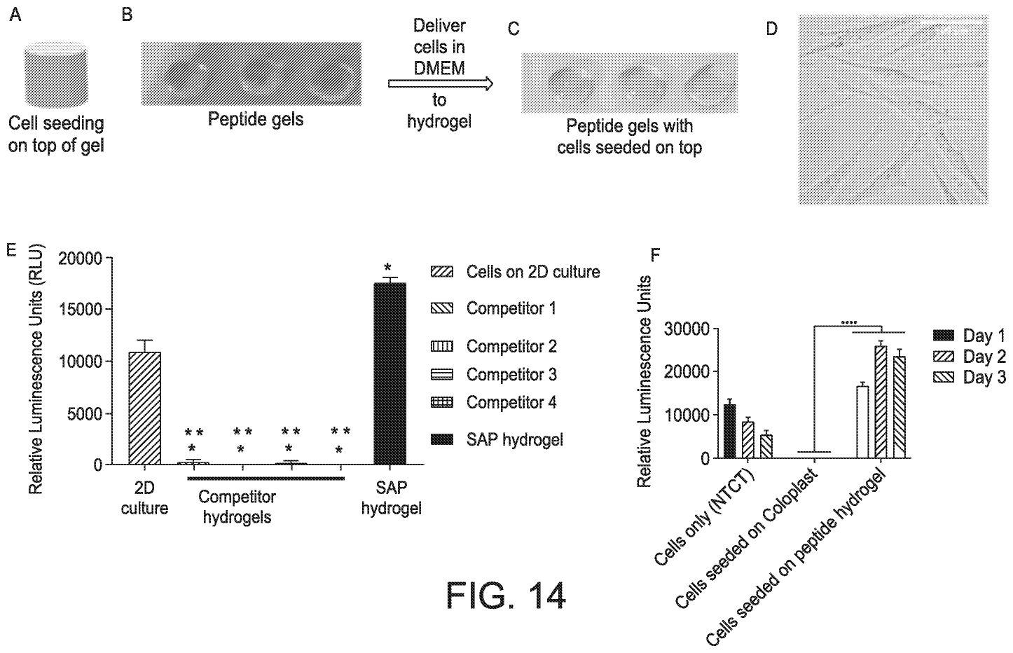

As shown in the images of FIG. 15, degradation rate of the hydrogel for targeted release of cells and/or agents may be controlled by peptide design. Briefly, peptide hydrogels of varying weight percentage were shown to deliver proteins of varying charge and varying protein size (kD) at different time points over a 30-day period. FITC-tagged proteins of varying size (20 kD, 70 kD and 150 kD) were delivered differentially from hydrogel administration over 2 weeks. Western blot shows that enzyme was released differentially from two different types of peptide gels over a 5-day period. The protein was not degraded.

The properties of the hydrogel may be selected to control a release profile of the biological material at the target site. For instance, the hydrogel may be engineered to provide rapid or immediate release of the biological material by designing a faster degradation profile. The hydrogel may be engineered to provide sustained or extended release of the biological material by designing a longer degradation profile. In certain embodiments, the administered hydrogel may comprise one or more layers of hydrogel having biological material therein. Each layer may be formed of a self-assembling peptide providing a selected degradation rate.

Exemplary layered hydrogels are shown in the schematic drawings of FIG. 11. The layered hydrogels may comprise two or more layers of hydrogel selected to have different properties, for example, degradation rate, pH, charge, etc. The layered hydrogels may comprise two or more layers of hydrogel having different additives, for example, cells, therapeutic agents, cell therapy agents, etc. In certain embodiments, cells may be substantially homogeneously suspended in one hydrogel and dispersed in clumps in a second hydrogel with different properties (as shown in image F of FIG. 11); cells may be substantially evenly dispersed in multiple hydrogels with differing degradation release properties, e.g., a first immediate release layer, a second rapid release layer, a third extended release layer, etc. (as shown in image G of FIG. 11); cells suspended in a first hydrogel having a first set of properties may be centered within a second hydrogel having a second set of properties (image H of FIG. 11), layered horizontally (image I of FIG. 11), or vertically (image J of FIG. 11).

In one exemplary embodiment, a first layer of the hydrogel, for example, an external layer in contact with the target tissue, may be designed to have the fastest degradation rate. Thus, the first layer may be designed or configured to release the biological material into the target site of the subject after a first predetermined period of time. A second layer of the hydrogel, for example, an internal layer in contact with the first layer, may be designed to have a second fastest degradation rate. Thus, the second layer may be designed or configured to release the biological material into the target site of the subject after a second predetermined period of time. The hydrogel may be designed to include additional consecutive layers, for example, third, fourth, fifth, sixth, seventh, eighth, ninth, tenth layers and so forth. Each layer having a different degradation rate and being designed or configured to release biological material into the target site of the subject at different predetermined periods of time. The biological material released by each hydrogel layer may be the same or different. In some embodiments, one or more hydrogel layers may be designed to release a combination therapy or other drug, with or without biological material. Such a gradient design may allow temporal regulation over the release of biological material to the target tissue.

Methods of producing a layered hydrogel having one or more classes of biological material are disclosed herein. The methods may comprise producing a first hydrogel layer having a first biological material suspended therein as previously described. The methods may comprise producing a second hydrogel layer adjacent the first hydrogel layer having a second biological material suspended therein. Each hydrogel layer may be formed by combining a self-assembling peptide having a selected degradation rate with a buffer and the biological material, as previously described. Additional hydrogel layers may be produced adjacent the first or the second hydrogel layer. In some embodiments, each hydrogel layer is produced to encapsulate a previous hydrogel layer.

In other embodiments, hydrogels having temporal regulation over the release of biological material may be designed by including a shell formed of a material having a slower degradation rate encapsulating each hydrogel layer. The degradation rate of the shell material may be selected to regulate timing of release of the biological material within each inner hydrogel layer. The methods of producing the layered hydrogel may comprise forming a shell layer adjacent to one or more hydrogel layer, for example, encapsulating one or more hydrogel layer. Exemplary shell layers may be formed of liposomes and/or Janus-faced molecules to confer biofunctionality and tunability to the layered hydrogel.

The hydrogel may be engineered to increase cytocompatibility with the biological material, while maintaining antimicrobial properties. In some embodiments, the biological material may be suspended in a hydrogel as previously described. The biological material hydrogel may be cytocompatible. The cytocompatible biological material hydrogel may be encapsulated with an antimicrobial hydrogel. For instance, a preparation comprising a self-assembling peptide configured to form an antimicrobial hydrogel may be combined with a buffer to form the antimicrobial hydrogel. The antimicrobial self-assembling peptide may have a net positive charge. For example, the antimicrobial peptide hydrogel may have a charge of +2 or greater, for example, up to +11, a charge of +2 to +9, or a charge of +5 to +9. The antimicrobial peptide preparation may be combined with the buffer on a surface of the cytocompatible hydrogel comprising biological material. Thus, the hydrogel may be formed of an interior hydrogel layer comprising biological material and an exterior hydrogel layer being antimicrobial. FIG. 11 includes schematic diagrams of multi-layered hydrogels.

Biofabrication of Cell Grafts with Peptide Hydrogels

Methods of preparing biological material grafts in vitro for administration in vivo are disclosed herein. The methods may include self-assembly of a preparation comprising biological material and a peptide into a hydrogel scaffold matrix in vitro. The self-assembled higher order structure may be administered to a target site of the subject.

The methods may comprise obtaining a biological material from a donor subject. The donor subject may be the recipient subject for autologous treatment or another subject for allogeneic or xenogeneic treatment. The methods may comprise suspending the biological material in the preparation comprising the peptide in a biocompatible solution. The methods may comprise combining the preparation with a buffer to induce self-assembly of the hydrogel, biofabricating the implantable sample.

In some embodiments, the methods may further comprise administering the hydrogel to a target tissue of the recipient subject.

In some embodiments, the biological material may be treated before administration to the recipient subject. Treatment may include, for example, one or more of, culturing, growing, activating, transducing, and expanding the biological material or cells for a period of time prior to administration. Treatment may include washing the biological material. Treatment may include combining the biological material with one or more additives or agents disclosed herein.

In some embodiments, the methods may include biofabrication of the biological material graft at a point of care. Thus, one or more of the steps for biofabrication may be performed at the point of care. In some embodiments, all of the steps may be performed at the point of care. For example, the biological material may be administered immediately after suspending or engrafting.

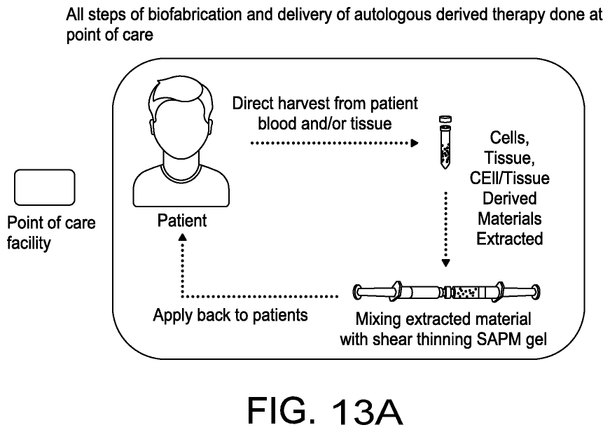

FIGS. 13A-13C are schematic diagrams of exemplary methods of biofabricating the self-assembling peptide hydrogel. FIG. 13A shows an exemplary method comprising performing all steps of biofabrication and administration of biological material at point of care. In the exemplary methods of FIG. 13A-13B the biological material is autologous. However, the biological material encapsulated and administered may be allogeneic or xenogeneic. FIG. 13B shows an exemplary method comprising harvesting the biological material from the subject at the point of care, engineering the biological material at an external facility, and biofabricating the biological material encapsulated in hydrogel at the point of care. FIG. 13C shows an exemplary method comprising harvesting the biological material from a donor subject and administering the biological material encapsulated in hydrogel to a recipient subject. FIGS. 13B-13C show engineering the biological material at an external facility. However, the biological material may be engineered or treated at the point of care. Additionally, the point of care for harvesting the biological material from a donor subject need not be the same point of care for encapsulating the biological material in the hydrogel and administering the biological material encapsulated in hydrogel to the recipient subject.

Mixing devices for biofabrication of cell grafts at a point of care are disclosed herein. The devices may include a first chamber for a cell preparation. The cell preparation may comprise cells suspended in water, media, or buffer. The devices may include a second chamber for a peptide preparation, and optionally a third chamber for a buffer, as previously described.

### Treatment of Nerve Injury

The peptide hydrogel preparations described herein have been found to provide a superior therapeutic effect in treatment of nerve injury. Certain properties of the peptide hydrogel have a synergistic effect on treatment of nerve injury. For instance, the anti-microbial effect of the peptide hydrogel may provide protection against pathogenic invaders; the peptide hydrogel may form a three-dimensional cryoprotective cell matrix locally at a target site; the peptide hydrogel may provide the ability to administer therapeutic agents on an extended release profile. Such properties of the hydrogel are believed to provide superior treatment of nerve injury.

Thus, in accordance with one aspect, there are provided methods of treating nerve injury. Nerve injuries may occur to any nervous tissue across the nervous system, e.g., central nervous system and peripheral nervous system. Injuries to the central nervous system include, for example, spinal cord injury (SCI), traumatic brain injury (TBI), and stroke. Exemplary injuries to the peripheral nervous system include peripheral nerve injury (PNI). While the description relates to spinal cord injury and peripheral nerve injury in exemplary embodiments, it should be noted that other nerve injuries may be treated by the methods disclosed herein.

The methods generally include administering the preparation to a target site of the nerve injury. The target site of the nerve injury may be associated with a central nervous system (CNS) tissue or a peripheral nervous system (PNS) tissue. In some embodiments, the target site may be a tissue selected from mesenchymal tissue, connective tissue, muscle tissue, nervous tissue, embryonic tissue, dermal tissue, bone tissue, dental tissue, corneal tissue, cutaneous tissue, integumental tissue, soft tissue, and hard tissue. The target site may be associated with a systemic effect. The target site may be associated with a desired local effect. In certain embodiments, the target site may be a nervous tissue. In some embodiments, the target site may be bone tissue, connective tissue, or muscle tissue, for example, associated with or proximate to the nervous tissue of the nerve injury.

The preparations disclosed herein may be administered in combination with a surgical treatment. The surgical treatment may be performed to remove compression of the spine. The surgical treatment may be performed to remove compression of an extremity, for example, an arm or a leg. For instance, the surgical treatment may be performed to remove fragments of bones, tendons, inflamed muscle tissue, foreign objects, herniated disks or fractured vertebrae. The surgical treatment may be performed to stabilize the spine and/or prevent future deformity. The surgical treatment may be performed to stabilize an extremity, for example, an arm or a leg. The preparations disclosed herein may be administered in combination with a stabilization treatment. For instance, the preparations may be administered in combination with immobilization of the neck, spine, back, arm, leg, and/or full body. The preparations may be administered in combination with physical therapy.

The preparations disclosed herein may be administered in combination with an approach commonly used to treat or approved to treat the nerve injury or a symptom of the nerve injury. Exemplary combination treatments include administration of growth factors, e.g., nerve growth factor (NGF), and/or neurotrophic factors, e.g., brain derived neurotrophic factor, ciliary neurotrophic factor, and glial derived neurotrophic factor (GDNF). Other combination treatments may be provided.

In accordance with another aspect, there is provided a method of preparing a nerve injury treatment composition. The method may comprise combining a therapeutically effective amount of a biological material suspension with the preparation and a buffer comprising an effective amount of an ionic salt and a biological buffering agent to form a hydrogel. The biological material suspension may comprise cells, cell-derived materials, tissue, and/or tissue-derived materials. In some embodiments, the method may comprise combining an anti-scarring agent with the composition. In some embodiments, the biological material suspension and the preparation may be combined first. In some embodiments, the biological material suspension and the buffer may be combined first. In yet other embodiments, the preparation and the buffer may be combined first forming the hydrogel. The biological material suspension may then be seeded onto the formed hydrogel. The anti-scarring agent may be combined with the biological material suspension, the preparation, or the buffer. Any one or more of the combinations may be made in vitro, in vivo, and/or in situ. The biological material may be cultured in the hydrogel for a predetermined period of time prior to administration.

In accordance with another aspect, there is provided a method of facilitating treatment of a nerve injury. The method may comprise providing the preparation, e.g., formulated for treatment of the nerve injury. For example, the preparation may be formulated for treatment of SCI. The preparation may be formulated for treatment of PNI. The method may additionally comprise providing instructions to combine the preparation with biological material. The instructions may describe an effective amount of the biological material to be combined with the preparation. The method may comprise providing instructions to combine the preparation with the buffer comprising an effective amount of an ionic salt and a biological buffering agent to form the hydrogel, as previously described.

The method may comprise providing instructions to agitate the hydrogel comprising the biological material. The hydrogel may be agitated to produce a non-homogeneous biological material suspension. For example, the hydrogel may be agitated to form clusters or spheroids of cells, cell-derived materials, tissue, and/or tissue-derived materials. In other embodiments, the hydrogel may be agitated to produce a substantially homogeneous suspension.

The method may comprise providing instructions to administer an effective amount of the hydrogel comprising the biological material to a target site of the nerve injury to provide treatment.

The method may additionally comprise providing instructions to combine the preparation with at least one combination treatment. For example, the method may comprise providing instructions to combine the preparation with an anti-scarring agent or any other combination treatment disclosed herein.

The method may comprise providing one or more of the biological material, the preparation, the anti-scarring agent, and/or the buffer. For example, the biological material suspension may be formulated for treatment of the nerve injury. The biological material suspension may comprise cells, cell-derived materials, tissue, and/or tissue-derived materials. The buffer may be formulated for treatment of the nerve injury. The method may comprise providing instructions to combine the preparation with at least one of the biological material, the anti-scarring agent, and the buffer in vitro, as previously described. The method may comprise providing instructions to combine the preparation with at least one of the biological material, the anti-scarring agent, and the buffer in vivo, as previously described.

The biological material may be autologous, allogeneic, or xenogeneic. Thus, the cells, cell-derived materials, tissue, and/or tissue-derived material may be autologous, allogeneic, or xenogeneic. The method may comprise obtaining the biological material from a donor. In certain instances, the method may comprise obtaining the biological material from the subject. The biological material may be prepared or treated for administration to a subject.

### Treatment of Spinal Cord Injury

In some embodiments, the nerve injury may be a spinal cord injury (SCI). Methods of treating spinal cord injury (SCI) are disclosed herein.

Recovery from injury to the mammalian spinal cord may be limited when severed axons are unable to regenerate. The inability of severed central nervous system (CNS) axons to re-grow has been attributed to many causative factors, including the physical impediment of the injury cyst/cavity, limited intrinsic regenerative capabilities, a multitude of inhibitory factors including myelin and chondroitin sulfate proteoglycans (CSPGs) and a lack of neurotrophic support. The heterogeneous nature of impediments is a challenge to developing effective treatments. However, experimental SCI models of cell grafting (e.g., to bridge the injury, provide a substrate for axon regrowth, produce axon-regenerative neurotrophic factors, decrease necrosis, improve cavity or void filling, and/or increase differentiation of progenitor cells), may provide a basis for effective acute and chronic SCI therapies.

The hydrogels disclosed herein may provide treatment to the injured spinal cord, including support of axon growth, migration, proliferation, and/or alignment, axon regeneration, re-innervation, attachment, reconnection, and/or remyelination, as well as provide tissue protection and replace lost neural tissue. The preparation may promote formation of a scaffold at the injury site, promoting cell attachment and/or cell migration. The preparation may promote bridging or void filling the injury site. The preparation may reduce or prevent scar formation at the injury site. Any one or more of these effects may provide treatment for the spinal cord injury.

The methods disclosed herein may treat acute, sub-acute, and chronic SCI. In certain exemplary embodiments, transplantation of Schwann cells, e.g., human Schwann cells, e.g., autologous human Schwann cells (ahSCs) may be feasible and safe for treatment of sub-acute thoracic subjects. Subjects having a degree of SCI ranging from complete injury to normal (A-E), as defined in the American Spinal Injury Association Impairment Scale (AIS), may be treated by the methods disclosed herein. For example, grade A, grade B, grade C, grade D, or grade E (AIS) injuries may be treated by the methods disclosed herein.

In particular, the methods described herein may provide safe and effective treatment of chronic thoracic and cervical SCI subjects. In certain embodiments, biological materials, e.g., cells, cell-derived materials, tissue, and/or tissue-derived materials, administered for treatment of SCI may originate from stem cells. The stem cells may include, for example, progenitor cells, multipotent cells, induced pluripotent cells, immune system cells, terminally-differentiated cells, de-differentiated cells, specialized cells, or combinations thereof. In certain exemplary embodiments, the stem cells may include bone marrow derived stromal cells and/or adipose derived stromal cells. The stem cells may be embryonic stem cells and/or adult stem cells.

In certain embodiments, biological materials, e.g., cells, cell-derived materials, tissue, and/or tissue-derived materials, administered for treatment of SCI may originate from glial cells. The glial cells may comprise oligodendrocytes, astrocytes, ependymal cells, microglia, Schwann cells, satellite cells, or combinations thereof.

In certain embodiments, the cell-derived materials and/or tissue-derived materials may comprise exosomes and/or lysosomes.

The methods may comprise administering the preparation and the buffer to a target site of the SCI. For example, the target site may be a central nervous system (CNS) tissue, as previously described. The preparation and/or buffer may be administered in an amount effective to treat the SCI. In accordance with certain embodiments, a biological suspension comprising may be administered in an amount effective to treat the SCI.

In certain embodiments, an effective amount of an anti-scarring agent may be administered to the target site in combination with the preparation and/or buffer. The anti-scarring agent may comprise at least one of receptor protein tyrosine phosphatase σ (RPTPσ) inhibitory peptide (ISP), chondroitinase ABC (ChaseABC), and polysialyl transferase (PST). The hydrogel and anti-scarring agent may exhibit synergistic effects in the treatment of SCI, in particular in combination with biological material, e.g., stem cells or glial cells. The glial cells may be cells located at the target site or cells administered with the composition. In particular, it is believed the glial cells and anti-scarring agent, when localized within or nearby the environment of the hydrogel, have a synergistic effect on treatment of SCI. The treatment may promote improved glial cell migration from the target site.

The preparation may be administered to a subject in need of treatment for SCI. For example, the subject may be diagnosed with SCI, e.g., by a licensed practitioner. In some embodiments, the SCI may be diagnosed based on a laboratory test and/or medical imaging. Spinal cord injury (SCI) includes any damage to the spinal cord. In general, SCI results in a loss of function, such as mobility and/or feeling.

Mild, moderate, or severe SCI may be treated by the methods disclosed herein. The SCI may be mild, for example, producing a mild to moderate discomfort for the subject. Mild SCI may include some loss of feeling. The SCI may be moderate, for example, producing moderate to severe discomfort for the subject. Moderate SCI may include loss of feeling, e.g., a debilitating loss of feeling. Moderate SCI may include loss of mobility. The SCI may be severe, for example, producing severe discomfort for the subject. Severe SCI may include severe loss of feeling, e.g., in a significant portion of the body, e.g., one or more extremities. Severe SCI may include loss of mobility. In some embodiments, severe SCI may include loss of mobility in a significant portion of the body, e.g., one or more limbs.

The methods may comprise treating incomplete or complete SCI. The SCI may be incomplete, e.g., the subject retains some motor or sensory function. A mild, moderate, or severe SCI may be incomplete. In other embodiments, the SCI may be complete, e.g., all feeling (sensory) and all ability to control movement (motor function) are lost. A complete SCI is generally considered severe. Examples of complete SCI include paraplegia (sensory and motor function loss of all or part of the trunk, legs, and pelvic organs) and quadriplegia (sensory and motor function loss of arms, hands, trunk, legs, and pelvic organs). Thus, paraplegia and quadriplegia may be treated by the methods disclosed herein.

The preparation may be administered in response to a symptom or trigger of SCI. For example, in some embodiments, the SCI may be caused or triggered by an injury, e.g., a traumatic injury. The injury may be a sudden blow affecting the CNS, e.g., the neck or spine. The injury may be a cut, puncture, or laceration affecting the CNS, e.g., the neck or spine. In some embodiments, the SCI may be caused or triggered by a disease or disorder. Exemplary diseases that may cause SCI include cancer and inflammation of the spinal cord. Exemplary diseases that may contribute to SCI include arthritis and osteoporosis. Symptoms of SCI include pain in the back or neck, pressure in the neck, head, or back, weakness, incoordination, paralysis, numbness, tingling or loss of sensation in the hands, fingers, feet, or toes, loss of bladder or bowel control, difficulty with balance and walking, impaired breathing after injury, oddly positioned or twisted neck or back.

The preparation may be administered to a target site associated with the SCI. The target site may be associated with a desired local effect, e.g., a local site of the injury. The preparation may be administered directly to at least a portion of the injured tissue. The target site may have soft tissue, hard tissue, or both. In some embodiments, the target site may be a local tissue of the injury selected from mesenchymal tissue, connective tissue, muscle tissue, nervous tissue, bone tissue, and combinations thereof.

The preparation may be administered to a target site associated with a desired systemic effect. The target site may have a tissue selected from mesenchymal tissue, connective tissue, muscle tissue, nervous tissue, embryonic tissue, dermal tissue, bone tissue, dental tissue, corneal tissue, cutaneous tissue, integumental tissue, soft tissue, hard tissue, and combinations thereof.

The preparation may be administered in combination with a surgical procedure. A surgical procedure may be performed on the subject to stabilize the spine or injured tissue and/or to remove matter compressing the spine or nervous tissue, such as bone fragments, tumors, or foreign matter. The preparation may be administered during the surgical procedure. For example, the preparation may be administered topically to an exposed internal tissue associated with the injury. In some embodiments, the preparation may be administered prior to stabilization of the spine or injured tissue. In some embodiments, the preparation may be administered after stabilization of the spine or injured tissue.

The preparation may be administered in combination with an approach commonly used to treat SCI or approved to treat SCI. The preparation may be administered in combination with an approach commonly used to treat a symptom of SCI or approved to treat a symptom of SCI. Exemplary combination treatments include hypothermia, medications to control pain and muscle spasticity, and/or medications that provide bladder control, bowel control, and control sexual functioning. Treatment may be provided to prevent deconditioning, muscle contractures, pressure ulcers, bowel and bladder issues, respiratory infections, and/or blood clots. The preparation may be administered in combination with rehabilitation, acupuncture, chiropractic treatment, and/or physical therapy.

The preparation may be administered in combination with an antibacterial composition, an antifungal composition, an antiviral composition, an anti-tumor composition, an anti-inflammatory composition, a cell culture media, a cell culture serum, an anti-odor composition, a hemostatic composition, and an analgesic or pain-relief composition.

As disclosed herein, Schwann cells (SC) may be administered for repair of the injured spinal cord. SCs have been shown to provide successful regeneration of peripheral nerves after injury and have demonstrated efficacy in a diverse range of acute and chronic experimental SCI paradigms. The SCs have favorable regenerative properties and ability to improve functional recovery which may provide feasibility, safety, and efficacy in human SCI.

Conventional methods of SCI treatment with cell transplantation often suffer from low Schwann cell (SC) survival within the injury milieu. For example, the survival rate of transplanted SCs within the injury milieu across diverse experimental SCI paradigms is about 15-20%. Because SCs have been known to associate with regenerating axons in a 1:1 ratio, it is believed that enhancing SC survival following transplantation should enhance the number of ingrowing axons after SCI and thus lead to more significant functional improvements.

The methods disclosed herein, including administering SCs with an implantable hydrogel matrix, may augment cell survival rate to almost 40% after SCI. Enhanced survival may be accompanied by substantial increases in the vascularization of the lesion, local and supraspinal axon growth, and/or significantly greater functional recovery. As disclosed herein, the preparations and methods may achieve successful SC transplantation by achieving ease of cell encapsulation and implantation within a scaffold that provides a high cell survival rate post-transplantation.

Conventional methods of SCI treatment with cell transplantation also suffer growth inhibitory effects of the glial scar on SC migration and long-tract axon growth support. In a fashion analogous to the dorsal root entry zone (DREZ), the glial scar that forms after SCI may restrict the outward migration of SCs from their intralesional deposition when transplanted. Glial scar tissue may prevent SCs from guiding retracted supraspinal axons, to help through the lesion and into the contiguous caudal cord, as well as prevent SCs from reaching areas of distal demyelination to mediate remyelination repair. The effectiveness of cell transplantation with SCs may be improved by the methods described herein, including encapsulating SCs in the cryoprotective hydrogel.

In certain embodiments, the use of the hydrogels disclosed herein may provide at least an 8-point motor score improvement with treatment in human SCI. In exemplary embodiments, the methods and preparations may provide broad utility in battlefield injuries to the nervous system as well as treatment of SCI in the civilian population.

In some embodiments, an effective amount of an anti-scarring agent may be administered to the target site in combination with the biological material. In some embodiments, cells, such as stem cells and/or glial cells, may be engineered to express an enzymatic anti-scarring agent, e.g., enzyme that overcomes the inhibitory effects of the glial scar. Exemplary anti-scarring agents include polysialyl transferase (PST) or chondroitinase ABC (ChaseABC). Engineered SCs may be able to migrate across the scar boundary, support serotonergic (5-HT) and corticospinal (CST) axon growth across the lesion, and/or provide improvements in functional recovery. Successful SCI recovery may be characterized then by the avoidance of inhibitory cues within the glial scar by growing axons and outward migrating cell transplants to provide functional bridging of the lesion.

The efficacy of the peptide hydrogel may be measured by histopathological and biochemical endpoints. In accordance with certain embodiments, the methods disclosed herein may provide SCI treatment for at least 3 months, for example, at least 6 months, at least 12 months, at least 18 months, or at least 24 months. The peptide hydrogel composition may be tested to examine the long-term (for example, at least 6 months) reparative and functional efficacy of the hydrogel combination with SCs against the current standard of media for cell suspension. Additionally, the combination of the peptide hydrogel with biological material in human SCI may provide an effective delivery agent for other cell and localized therapeutics targeting CNS repair.

In accordance with some embodiments, the peptide hydrogel may be engineered to provide glial scar avoidance properties and re-grow axons and SCs for 1 month after SCI by peptide functionalization. In accordance with some embodiments, the peptide hydrogel may provide long-term (for example, at least 6 months) treatment properties, such as cell persistence, CNS distribution, axon growth support, remyelination, and/or functional efficacy of encapsulated cells when transplanted into the injured spinal cord and/or cells located at the target site.

The preparation, buffer, anti-scarring agent, and/or biological material may be delivered by a minimally invasive procedure. For example, the preparation, buffer, anti-scarring agent, and/or biological material may be delivered parenterally for treatment of SCI. In certain embodiments, the preparation, buffer, and/or biological material may be delivered by intraspinal delivery. In accordance with some embodiments, the peptide hydrogel formulation may be engineered for biological material encapsulation during intraspinal delivery to the injured spinal cord, to improve or maximize in vivo cell survival rates.

### Treatment of Peripheral Nerve Injury

In some embodiments, the nerve injury may be a peripheral nerve injury (PNI). Methods of treating peripheral nerve injury (PNI) are disclosed herein.

The methods may comprise administering the preparation and the buffer to a target site of the PNI. For example, the target site may be a peripheral nervous system (PNS) tissue, as previously described. The preparation and/or buffer may be administered in an amount effective to treat the PNI.

In accordance with certain embodiments, a biological material suspension may be administered in an amount effective to treat the PNI. The biological material may include cells, cell-derived materials, tissue, and/or tissue-derived materials. The biological material may be derived from stem cells. Exemplary stem cells that may be administered to treat PNI include neural stem cells, adipose-derived stem cells, bone marrow mesenchymal stem cells, muscle-derived stem cells, hair follicle stem cells, dental pulp stem cells, skin-derived stem cells, or induced pluripotent stem cells. The stem cells may be terminally-differentiated cells, de-differentiated cells, specialized cells, or combinations thereof. In certain exemplary embodiments, the stem cells may include bone marrow derived stromal cells and/or adipose derived stromal cells. The stem cells may be embryonic stem cells and/or adult stem cells.

The biological material may be derived from glial cells. Exemplary glial cells that may be administered to treat PNI include oligodendrocytes, astrocytes, ependymal cells, microglia, Schwann cells, satellite cells, and combinations thereof, as previously described. The cells may be Schwann cells (SC) as previously described.

In certain embodiments, the cell-derived materials and/or tissue-derived materials may comprise exosomes and/or lysosomes.

In some embodiments, an effective amount of an anti-scarring agent may be administered to the target site in combination with the biological material. In some embodiments, the biological material, for example, cells, may be engineered to express an enzymatic anti-scarring agent, as previously described.

Conventionally, neural stem cells (NSC) and Schwann cells (SCs) have been administered encapsulated in matrigel or other gels. The self-assembling hydrogels disclosed herein show properties not found in other products. For instance, matrigel is extracted from Engelbrth-Holm-Swarm (EHS) mouse sarcoma and is tumorigenic. Thus, it may not be ideal for use in humans. Matrigel also conventionally comprises growth factors. Thus, further modifications of the biological material may be required for compatibility with growth factors in the matrigel matrix. Collagen has a slower rate of gelation, which may contribute to undesirable cell transplantation losses. Alginate gels conventionally require the addition of side groups to permit cell attachment, which may need modifications for functionality. The self-assembling peptide hydrogel descried herein has superior properties to these conventional gel materials, as previously described.

In accordance with certain embodiments, the peptide hydrogel may provide a beneficial microenvironment for the improvement of cell survival, axon growth and remyelination when administered to the target site of the injury. Administration of the hydrogel and stem cells may provide improved preservation of motoneurons, skeletal muscle fibers, and neuromuscular junction tissues when administered to the target site of a PNI. The hydrogel and stem cells may provide a synergistic effect in the treatment of PNI, for example, the hydrogel may improve the functional efficacy of encapsulated stem cells transplanted in peripheral nerves post-acute or chronic injury for treatment of PNI. Thus, the injury to be treated may be acute. The injury to be treated may be chronic.

The hydrogels for treatment of PNI may additionally comprise one or more component to enhance axon growth, migration, proliferation, and/or alignment, axon regeneration, re-innervation, and attachment to the motor endplate. The preparation may promote formation of a scaffold at the injury site, promoting cell attachment and/or cell migration. The preparation may promote bridging or void filling the injury site. The preparation may reduce or prevent scar formation at the injury site. Any one or more of these effects may provide treatment for the PNI.

In certain embodiments, administered cells may be engineered to express one or more component to enhance axon regeneration, re-innervation, and attachment to the motor endplate. Exemplary components comprise brain-derived neurotrophic factor (BDNF), glial cell line-derived neurotrophic factor (GDNF), brain-derived neurotrophic factor (BDNF), vascular endothelial growth factor (VEGF), calcium, neurotrophin-3, IL-1, neuregulin, GAP-43, tubulin, actin, heat-shock protein-27 (HSP-27), and combinations thereof. The hydrogel and component may exhibit synergistic effects in the treatment of PNI, in particular in combination with stem cells and/or glial cells. The stem cells may be administered with the composition. The glial cells may be cells located at the target site or cells administered with the composition.

The preparation may be administered to a subject in need of treatment for PNI. For example, the subject may be diagnosed with PNI, e.g., by a licensed practitioner. In some embodiments, the PNI may be diagnosed based on a laboratory test and/or medical imaging. Peripheral nerve injury (PNI) includes any damage to the peripheral nerves. In general, PNI results in a neurological deficit, such as loss of mobility and/or feeling, distal to the site of the injury.

Although peripheral nerve injuries are rarely life-threatening, such injuries impact a patient's quality of life and may produce permanent muscle degeneration. Repair of a peripheral nerve without restoration of attachment of the nerve to a neuromuscular junction (NMJ) may result in incomplete functional recovery. Damage to connective tissues surrounding the injured nerve may result in scar tissue formation. Scar tissue formation may inhibit proper growth cone formation resulting in suboptimal recovery. Damage to muscle tissue surrounding the damaged peripheral nerve can also result in muscle fibrosis, leading to permanent losses. The methods disclosed herein may be employed to inhibit, limit, or reduce scar tissue formation, improper growth cone formation, and/or muscle fibrosis.

Mild, moderate, or severe PNI may be treated by the methods disclosed herein. The PNI may be mild, for example, producing a mild to moderate discomfort for the subject. Mild PNI may include some loss of feeling, e.g., reversible partial or complete loss of feeling. The PNI may be moderate, for example, producing moderate to severe discomfort for the subject. Moderate PNI may include some damage to axons. Moderate PNI occasionally requires a procedure, e.g., surgery, for repair. The PNI may be severe, for example, producing severe discomfort for the subject. Severe PNI may include damage to the axons which prevents passage electrical signals. Severe PNI generally requires a procedure, e.g., surgery, for repair.

The methods may comprise treating first, second, third, fourth, or fifth degree PNI. First degree PNI may be characterized by a reversible local conduction block at the site of the injury. Second degree PNI may be characterized by a loss of continuity of the axons. Third degree PNI may be characterized by damage to the axons and supporting structures within the nerve. Fourth degree PNI may be characterized by damage to the axons and the surrounding tissues sufficient to create scarring that prevents nerve regeneration. Fifth degree PNI may be characterized a split or division of the neural tissue. With fourth and fifth degree PNI, electrical energy is generally prevented from passing along the neural pathways.

Peripheral nerve injuries may result in the loss of sensation or strength in the upper or lower extremities. The peripheral nerves that tend to have the highest risk for injury are nerves with a superficial location, nerves that extend for long distances through the body, and nerves that pass through narrow, bony canals. The PNI may be a carpal tunnel syndrome or ulnar nerve injury. Peripheral nerve injuries that impact the upper extremities include carpal tunnel syndrome and cubital tunnel syndrome. Carpal tunnel syndrome, caused by entrapment of the median nerve at the wrist, occurs in approximately 5-15% of persons in an industrialized setting. Ulnar nerve entrapment injuries such as cubital tunnel syndrome impact a disproportionate number of young to middle-aged males in the lower-income bracket, and occasionally require hospitalization. Symptoms that begin with paresthesia and numbness often progress over time to muscle weakness and debilitating wasting, resulting in loss of productivity and increased health care costs.

The PNI may be a sciatic nerve injury or peroneal nerve injury. Peripheral nerve injuries that impact the lower extremities include sciatica and peroneal nerve injuries. Peroneal nerve injuries are a common lower extremity nerve injury, especially among males between 16-59 years of age, and often resulting from acute trauma such as motorcycle or car accidents and other blunt limb damage. Peroneal nerve injuries are often associated with immobilization, extended hospitalization and rehabilitation treatment, and increased disability from employment.

Sciatic nerve injuries are common PNIs of the lower extremity that often have insidious onset and a chronic nature. The sciatic nerve originates in the pelvic cavity, passes through the biceps femoris, and terminates at the posterior knee. Due to the length of the sciatic nerve, sciatica is a common injury which occurs whenever any part of the nerve is injured. The most common symptoms include pain in the lumbar spine and a burning sensation deep in the buttocks with associated paresthesia, weak knee flexion, and foot drop. Although 50-70% of sciatica patients experience short-term improvements following surgery, 10-40% of patients experience recurrence of sciatica over the long-term, and few patients experience full functional recovery. Many patients remain dependent on pain medications, are disabled from employment, and experience a poor quality of life.

The PNI may be an acute, sub-acute, or chronic injury. Chronic peripheral nerve injuries are often associated with degradation of the myelin sheath, decreased internodal length, increased Schwann cell metabolism, increased development of fibrous and scar tissue around the nerve associated with intraneural edema, and/or a gradual decline in nerve conduction velocity. While not wishing to be bound by theory, it is believed that following neuronal damage, Schwann cells (SCs) play a role in degeneration of the distal neural stump to pave the way for regeneration of the axon, in cytoskeletal disintegration, and in the recruitment of macrophages via monocyte chemoattractant protein-1 (MCP-1) to clear debris. In the absence of axonal contact, such as when an axon is severed, Schwann cells have been found to convert to a de-differentiated, demyelinating phenotype and downregulate the expression of PMP22, Krox-20, P0 and connexin-32. Schwann cell activity may be regulated by changes in the expression of integrin B4. After clearance of neuronal debris, Schwann cells tend to proliferate on the extracellular matrix to form hollow tubes through which axon regeneration can occur. The tubes may atrophy if no axon regeneration occurs within the tube.

At the proximal stump, neurons may release ATP and neuregulin, which help to mature Schwann cells to a myelinating, regeneration phenotype. Regeneration of the damaged axon may occur under the control of proteins such as GAP-43, tubulin, actin, and heat-shock protein-27 (HSP-27). Regeneration proteins may be administered in combination with the preparations disclosed herein. Additionally, in the absence of regeneration of the injured axon, functional recovery may still occur when remaining intact neurons branch to newly-innervated muscles to compensate for atrophied sections. Neuronal branching and survival may be regulated by neurotrophic factors such as brain-derived neurotrophic factor (BDNF), glial cell line-derived neurotrophic factor (GDNF), ciliary neurotrophic factor (CNF), and neurotrophin-3. Neurotrophic factors may be administered in combination with the preparations disclosed herein.

The methods disclosed herein include promoting growth cone formation. Growth cone formation at the proximal nerve bud may be affected by calcium, actin, and myosin. Receptors on the surface of cells in the growth cone may respond positively to guidance molecules such as semaphorins, ephrins, netrins, and slits, and negatively to inhibitory guidance molecules such as Collapsin-1. Additional regulation may be provided by neurotrophins such as brain-derived neurotrophic factor (BDNF), which decrease the sensitivity of receptors to guidance molecules. Schwann cells may be employed by the methods disclosed herein to provide neurotrophic factors, neurite-promoting factors, and other regulatory molecules described above. In certain embodiments, one or more regulatory molecule described above may be administered in combination with the preparation. Macrophages may help promote peripheral nerve regeneration by secreting IL-1 to increase the production of neuronal growth factors (NGF) and the expression of NGF receptors on Schwann cells, driving Schwann cell proliferation. However, macrophages may also inhibit Schwann cells by secreting IL-1 receptor antagonist. Thus, peripheral nerve injury recovery may be promoted by controlling expression or secretion of regulatory molecules, such as IL-1, NGF, NGF receptors, and IL-1 receptor antagonist.

The preparation may be administered in response to a symptom or trigger of PNI. For example, in some embodiments, the PNI may be caused or triggered by an injury, e.g., a traumatic injury. The injury may be a sudden blow affecting the PNS. The injury may be a cut, puncture, or laceration affecting the PNS. The injury may cause damage to the PNS in the form of stretching, compressing, crushing, or severing nervous system tissue.

In some embodiments, the PNI may be caused or triggered by a disease or disorder. Exemplary diseases that may cause PNI include diabetes, Guillain-Barre syndrome and carpal tunnel syndrome. Autoimmune diseases that may cause or contribute to PNI include lupus, rheumatoid arthritis, and Sjogren's syndrome. Other diseases and disorders that may contribute to PNI include narrowing of the arteries, hormonal imbalances, cancer, e.g., tumors, and osteoporosis.

The methods disclosed herein may be used for treatment of damage to motor nerves or a symptom thereof. Symptoms of PNI that may be associated with damage to motor nerves include muscle weakness, cramps, and muscle twitching, e.g., uncontrollable muscle twitching. The methods disclosed herein may be used for treatment of damage to sensory nerves or a symptom thereof. Symptoms of PNI that may be associated with damage to sensory nerves include numbness or tingling, e.g., in extremities such as hands or feet, trouble sensing pain or changes in temperature, trouble walking or keeping balance, e.g., without eyesight, or trouble performing manual tasks, such as fastening buttons. The methods disclosed herein may be used for treatment of damage to autonomic nerves or a symptom thereof. Symptoms of PNI that may be associated with damage to autonomic nerves include excessive sweating, changes in blood pressure, inability to tolerate temperature changes, such as heat or cold, and gastrointestinal symptoms. Injuries associated with damage to more than one type of nerve may also be treated. For instance, a variety of symptoms may be experienced by the subject because PNI may affect more than one type of nerve fiber.

The preparation may be administered to a target site associated with the PNI. The target site may be associated with a desired local effect, e.g., a local site of the injury. The preparation may be administered directly to at least a portion of the injured tissue. The target site may have soft tissue, hard tissue, or both. In some embodiments, the target site may be a local tissue of the injury selected from mesenchymal tissue, connective tissue, muscle tissue, nervous tissue, bone tissue, and combinations thereof.

The preparation may be administered to a target site associated with a desired systemic effect. The target site may have a tissue selected from mesenchymal tissue, connective tissue, muscle tissue, nervous tissue, embryonic tissue, dermal tissue, bone tissue, dental tissue, corneal tissue, cutaneous tissue, integumental tissue, soft tissue, hard tissue, and combinations thereof.

The preparation may be administered in combination with a surgical procedure. A surgical procedure may be performed on the subject to stabilize a region of the injury and/or to remove matter compressing the nervous tissue, such as bone fragments, tumors, or foreign matter. A surgical procedure may be performed to treat the injured tissue, e.g., surgically reattach severed nervous tissue. The preparation may be administered during the surgical procedure. For example, the preparation may be administered topically to an exposed internal tissue associated with the injury. In some embodiments, the preparation may be administered prior to the stabilization or reattachment of tissue. In some embodiments, the preparation may be administered after the stabilization or reattachment of tissue.

Treatments for PNI may include microsurgical repair of injuries less than 5 mm in length and autologous nerve grafting for injuries longer than 3 cm in length. Administration of the preparations disclosed herein may improve functional recovery when combined with another treatment. Other treatments which may be supplemented with administration of the preparation include administration of donor sensory nerves for repair of damaged motor nerves, allografting biological or synthetic nerve conduits seeded with Schwann cells and neurotrophic factors, optionally with immunosuppressive therapy, and other cell-based therapies to enhance neural regeneration by increasing axonal regeneration, re-myelination, and maintaining muscle mass.

The preparation may be administered in combination with an approach commonly used to treat PNI or approved to treat PNI. The preparation may be administered in combination with an approach commonly used to treat a symptom of PNI or approved to treat a symptom of PNI. Exemplary combination treatments include physical stabilization, e.g., braces or splints, electrical stimulation, hypothermia, medications to control pain and muscle spasticity, and/or medications that provide bladder control, bowel control, and control sexual functioning. Treatment may be provided to prevent deconditioning, muscle contractures, pressure ulcers, bowel and bladder issues, respiratory infections, and/or blood clots. The preparation may be administered in combination with rehabilitation, acupuncture, chiropractic treatment, and/or physical therapy.

The preparation may be administered in combination with an antibacterial composition, an antifungal composition, an antiviral composition, an anti-tumor composition, an anti-inflammatory composition, a cell culture media, a cell culture serum, an anti-odor composition, a hemostatic composition, and an analgesic or pain-relief composition.

### EXAMPLES

The function and advantages of these and other embodiments can be better understood from the following examples. These examples are intended to be illustrative in nature and are not considered to be limiting the scope of the invention.

### Example 1: Functionalized Peptide Hydrogels in Varying Concentrations of Peptide Matrix (0.25%, 0.5%, 0.75%, 1.5% w/v)

The peptide hydrogels may be functionalized to exhibit glial scar avoidance properties for growing axons and migrating Schwann cells (SC).

Transplanted SCs, irrespective of injury type or the timing of transplantation, are typically unable to migrate from their original site of deposition within the lesion into the adjacent host spinal cord after spinal cord injury (SCI). The inability of SCs to migrate from the lesion prevents them from reaching retracted axons rostral to the lesion, so as to guide them through the injury, as well as assist in the re-entry of axons from the lesion to their neuronal targets in the contiguous caudal spinal cord. Restricted SC migration also limits the ability of transplanted cells to reach areas of distal demyelination to enact remyelination repair and restore axonal conduction. The glial scar forms the primary impediment to SC migration in a fashion analogous to the dorsal root entry zone (DREZ). Astrocytes and the inhibitory chondroitin sulfate proteoglycan (CSPG) matrix they produce therefore comprise both a physical and chemical barrier. The same barrier is also the major extrinsic inhibitor of axonal regeneration after SCI. Blocking or degrading the inhibitory CSPGs, such as with chondroitinase ABC (ChaseABC) after SCI, can both facilitate the outward migration of transplanted SCs as well as relieve the inhibition on axon regeneration, leading to improvements in functional recovery after SCI.

Alternatively, blocking cell to cell interactions or axon to cell interactions after SCI by enhancing surface prostate-specific antigen (PSA) can also allow SCs to migrate from the lesion site as well as support axon regeneration and improved function. Identification of receptor protein tyrosine phosphatase σ (RPTPσ), the receptor for CSPGs in both axons and SCs has provided another strategy for imbuing scar-avoidance activity to axons or cells. Introduction to the spinal cord after injury of a cell-permeable RPTPσ inhibitory peptide (ISP), which blocks the intracellular signaling activity of the receptor, has been demonstrated to enable supraspinal axons to cross the injury site in large numbers and restore function after SCI.

The peptide hydrogels may be functionalized with ISP or ChaseABC enzymes to enable the material to imbue host axons and transplanted SCs with scar avoidance activity. The functionalization is expected to significantly enhance the reparative action of the SC-matrix and lead to improvements in functional recovery.

The long-term drug release profile of the peptide hydrogel functionalized matrices with the two CSPG targeted agents will be confirmed in vitro. An effective concentration of the peptide by weight percent will be used such that 20% of the enzyme or peptide is retained in the peptide matrices at 28 days. The long-term release of ISP or ChaseABC will either provide scar avoidance behavior to cells and axons or degrade inhibitory CSPGs, respectively, for a length of time sufficient for SCs to migrate outward from the lesion and axons to grow across the lesion-transplant into the contiguous caudal spinal cord.

Self-assembling peptide scaffolds will be functionalized with loading anti-scarring agents according to a previously established drug loading and release study protocol. A total of six treatment groups: final concentration of 5 μM RPTPσ inhibitory peptide ISP and 2 U/100 μl ChaseABC in varying concentrations of peptide matrix (0.25% w/v-3% w/v) will be tested. Respective anti-scarring agents will be reconstituted in peptide stocks and samples will be carefully shaken to initiate hydrogelation, resulting in gels with final concentrations of 0.25%, 0.5%, 0.75%, 1.0%, 1.25%, 1.5%, 1.75%, 2.0%, 2.25%, 2.5%, 2.75%, and 3% w/v and a total gel volume of 300 μL containing the agents.

After functionalizing the gels, gels will be dispensed into glass vials through a syringe to simulate intraspinal application, with 3 ml of buffer (100 mM BTP, pH 7.4 containing 300 mM NaCl) placed on top of gels and plated at 37° C. At scheduled time points 1, 7, 14, 21 and 28 days, the entire volume of buffer supernatant above the gel will be removed and replaced with fresh buffer. Probe concentration will be determined for each removed aliquot as a function of time. Each time point will be performed in triplicate. Concentrations of ISP and ChaseABC released as well as that retained in the gels will be quantified following established protocols on peptides (MS-ESI (+)) and protein release from peptide gels.

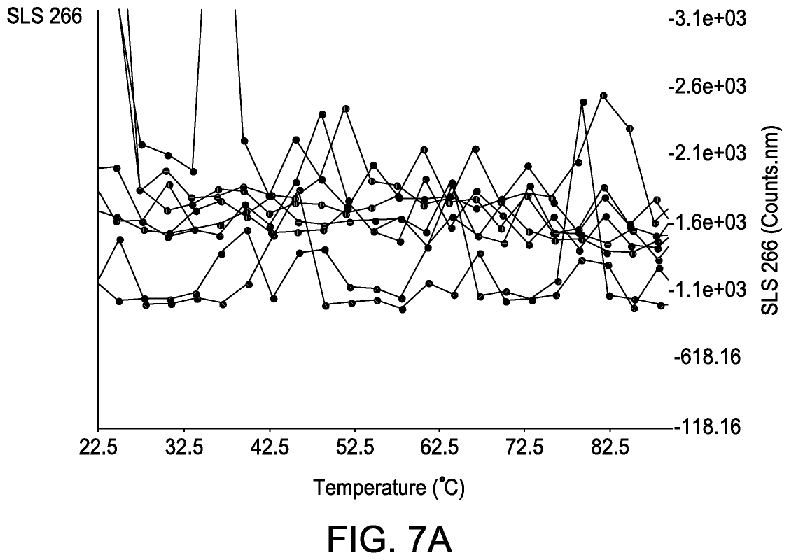

In addition to release and retention rates, in analogous experiments the released and retained ChaseABC enzyme will also be assayed for bioactivity. Activity will be assessed through the efficacy of the sample to digest the small proteoglycan decorin as described previously. In pilot data, the feasibility of ChaseABC encapsulation and release of bioactive enzyme from the gel was demonstrated (see, e.g., FIGS. 6A-6B). FIGS. 6A-6B show controlled release of therapeutics from peptide gels. Graph (A) of FIG. 6A shows the peptide gel delivery model for proteins of different charges for over 30 days. Graph (B) of FIG. 6A shows how peptide gel matrices of varying weight percentages deliver proteins of varying protein size over 30 days. Image (C) of FIG. 6A shows FITC tagged proteins of varying size. FIG. 6B shows the results of SDS page of ChaseABC release from gels, demonstrating the bioactivity of enzyme on decorin substrate at days 3 and 5.

The experiment will identify an effective peptide gel concentration that provides 28 days of peptide and enzyme release with 20% retention within gels.

### Example 2: In Vivo Administration of Functionalized Peptide Hydrogels

The same SCI model employed in example 1 will be used for in vivo testing. The effective composition of peptide hydrogel with superior cytoprotective action as determined from the tests in example 1 will be used for all treatment groups employing the combination of SCs and matrix in this experiment. Comparative control groups will use SC transplantation in media (standard protocol).

Adult female Fischer rats will receive contusive C5 cervical SCI and the transplantation of SCs and/or peptide matrix. The peptide hydrogel will be functionalized to contain one of two agents to provide scar-avoidance activity to injured axons and SCs: ChaseABC or the RPTPσ inhibitory peptide ISP. The enzyme or peptide will be encapsulated in the hydrogel for extended delivery during the biodegradation of the gel, a period of approximately 28 days. Control groups consisting of SCs alone, hydrogel alone, or the combination of SCs in media with enzyme or peptide, will be employed.

Each group will be divided into two cohorts for either biochemical or histological analysis. To determine the effectiveness of the CSPG therapies on their specific targets over time, one cohort of animals (n=15) will be sacrificed at 3, 10 or 20 days (n=5 per time point) following SC transplantation to examine changes in levels of CSPG core protein (CS-56), digested GAG (2B6 antibody) as well as Cathepsin B and IL-10 (two correlate biomarkers that have been shown to exhibit robust increases in expression with RPTPσ ISP delivery) by western blotting and ELISA within the lesion-transplant (5 mm length). In these same samples, as well as the rostral and caudal cord (5 mm segments) immediately adjacent, levels of neuronal/axonal markers (NF, 5-HT, DβH, SubP, CGRP and ChAT) will also be analyzed by immunoblot.

In the second cohort of animals in each group (n=10), at 20 days after SC transplantation, fixed spinal cord tissue samples will be sectioned to evaluate SC survival (GFP cell counts), graft volume, SC proliferation (PCNA and Ki67) and SC apoptosis (APOPTAG-Tunnel) as well the extent of SC migration into the adjacent rostral and caudal host tissue. Immunohistochemistry will be employed to confirm persistent changes in CSPG levels and degradation as well as measure the density of axonal growth (NF, 5-HT, DβH, SubP, CGRP and ChAT) into the lesion-transplant and within the caudal cord. The effectiveness of the scar-avoidance therapies will be determined by changes in either CSPG glycosaminoglycan (GAG) cleavage (2B6) or correlative proteins (Cathepsin B and IL-10).

The peptide hydrogel may be functionalized to improve the load, stability and/or release kinetics of the enzyme or peptide. The primary measure of efficacy will be an enhancement of 5-HT axon density within and caudal to the lesion-transplant compared to the SC only control group. ChaseABC and ISP have been demonstrated to greatly enhance 5-HT axon growth after SCI. The treatment group with the most significant increase in 5-HT density may be further evaluated for the ability of this combinatory approach to further improve functional recovery after SCI compared to SCs alone will be assessed.

The peptide hydrogels disclosed herein may provide the advantage of a tunable biodesign matrix technology. Exemplary approaches for prolonged delivery of the functionalized group may be to incorporate the tethering of the TAT peptide with a cleavable linker (e.g. Glutamate acid), or the use of alternative molecules, such as siRNA against CSPG synthesizing enzymes in the peptide hydrogel to relieve CSPG inhibition of axon growth and SC migration.

### Example 3: Modification of Peptide Hydrogels Increases Cytocompatibility with Cells

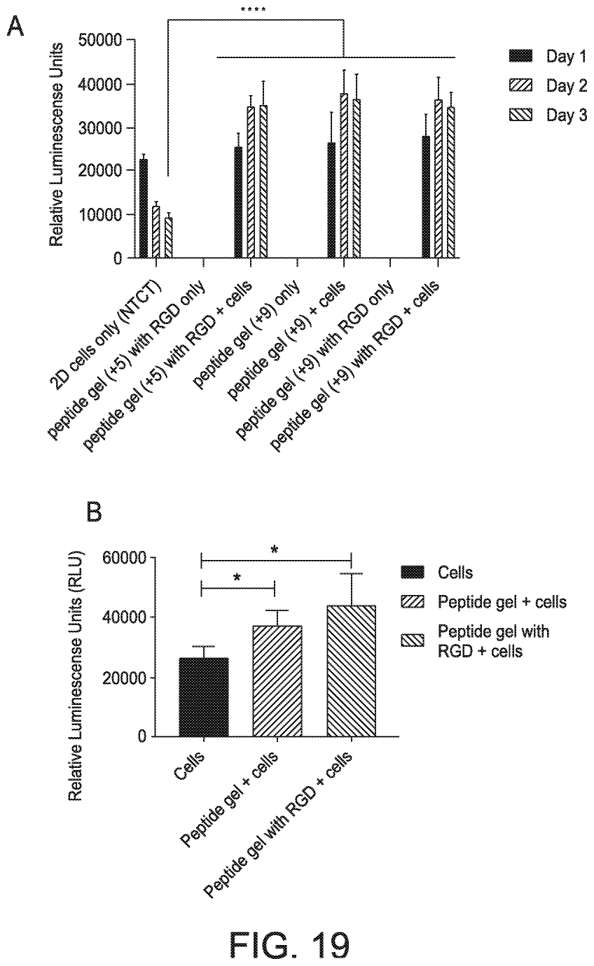

Peptide hydrogels were modified with RGD, VEGF, Thym1/2. Mesenchymal stem cells (MSCs) were seeded on top of the hydrogel. The modified hydrogels significantly increased cytocompatability (n=4) (p<0.0001) over a two-dimensional cell culture. The results are shown in image A of FIG. 19.

Human retinal pigment epithelial (RPE) cells were also seeded on top of the modified hydrogel (n=3). The modified hydrogel significantly increased viability of the RPE cells compared to unmodified peptide hydrogels (p<0.05), as measured by relative luminescence units (RLU) using a standard luciferase viability assay. The results are shown in image B of FIG. 19.

Schwann cells (SCs) were also seeded on top of the hydrogel and modified peptide hydrogel. The hydrogel culture also demonstrated higher viability than a two-dimensional cell culture (p<0.05). The modified hydrogel demonstrated even higher viability on than the hydrogel (p<0.05). The results are shown in image D of FIG. 19. Fluorescence images show live (green) RPE cells on a two-dimensional culture compared to RGD-modified hydrogels (image C of FIG. 19) as well as Schwann cells on non-tissue culture treated (NTCT) and TCT plates compared to unmodified and modified hydrogels (image E of FIG. 19). Cells on the hydrogel also showed a healthy spreading morphology.

### Example 4: Peptide Hydrogel Allows Controlled Release of Therapeutics

Peptide hydrogels of varying weight percentage were prepared to deliver proteins of varying charge and varying protein size (kD) over a 30-day period. Briefly, FITC-tagged proteins of varying size (20 kD, 70 kD, and 150 kD) were delivered differentially in a peptide hydrogel over 2 weeks. Western blot analysis (FIG. 15) showed that ChaseABC, measured by the presence or absence of decorin (˜50 kDa), was released differentially from two different types of peptide hydrogels over a 5-day period. Importantly, the protein was not degraded.

### Example 5: Schwann Cells Exhibit Enhanced Cell Viability and Proliferation when Cultured on Self-Assembling Peptide Hydrogel Matrix

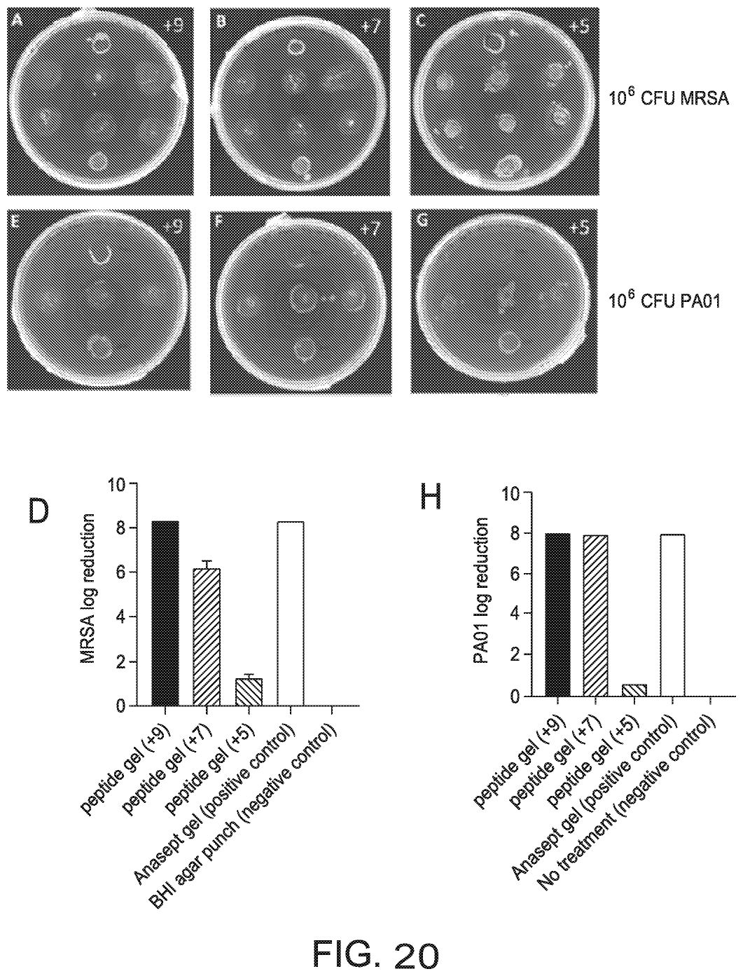

Schwann cells were cultured 24 hours on the surface of a peptide hydrogel and a peptide hydrogel modified with an anti-apoptotic motif. Viability and proliferation of the cell cultures were measured. The results are shown in the graph and fluorescence images of FIG. 21. Briefly, the peptide hydrogel culture exhibited enhanced viability and proliferation (p<0.05, bottom right image) over a standard tissue culture control plate. The modified peptide hydrogel exhibited further enhanced viability and proliferation (p<0.05, top right image).

### Example 6: Rodent Model of Peripheral Nerve Injury Treatment

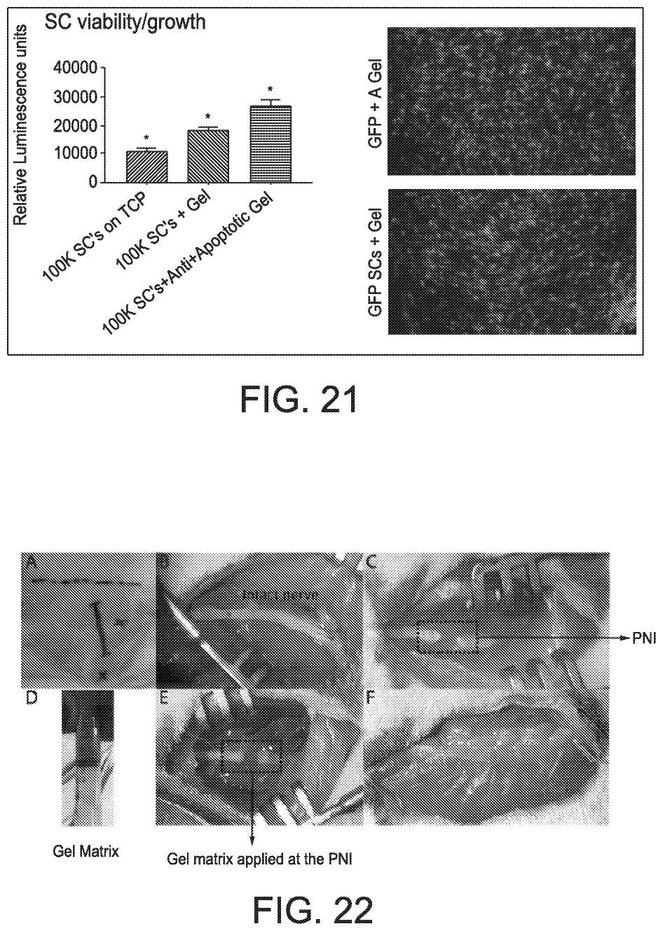

An incision was made to a peripheral nerve in a rodent model. Image A of FIG. 22 shows the incision site prior to incision. Image B of FIG. 22 shows the intact nerve prior to incision. Image C of FIG. 22 shows the post-incision peripheral nerve injury.

The injury was surgically re-sectioned with the peptide hydrogel. Image D of FIG. 22 shows the peptide hydrogel prior to application. Image E of FIG. 22 shows application of the peptide hydrogel at the peripheral nerve injury site. Image F of FIG. 22 shows the surgically re-sealed peripheral nerve injury. As shown in the images, the peptide hydrogel applied to the injury site may enhance structural integrity of the peripheral nerve and provide axon regeneration and improved functional recovery.

### Example 7: Schwann Cell Transplantation for Spinal Cord Injury Repair

It has been shown that CNS axons may regenerate if provided a suitable environment. For instance, the peripheral nerve environment may foster regeneration of axons from neurons in the CNS. Schwann cells (SC) are a component of the peripheral nerve environment, commonly performing either ensheathment or myelination of all axons in the mammalian PNS. Thus, SCs may be a supportive cell promoting axonal regeneration after peripheral nerve damage. In particular, autologous human SCs (ahSCs) may be used in the repair and restoration of function for long peripheral nerve deficits in humans that do not undergo endogenous repair. SCs may secrete a variety of growth factors and extracellular matrix (ECM) proteins that support growing axons and/or can prevent injury-induced cell death and tissue damage. SCs may also possess the ability to myelinate axons and restore axon conduction.

Thus, the potential of SC transplantation to mediate repair after SCI was investigated. In a wide diversity of SCI paradigms, acute and chronic, thoracic and cervical, complete and incomplete, SC transplants may survive long-term, provide significant protection of host tissue adjacent to the lesion, support robust growth of axons including supraspinal axons, enact substantial remyelination of in-growing axons and lead to improvements in axon conduction across the lesion as well as significant functional recovery. SCs may provide reparative action after SCI with cells and cell-derived material from multiple species, for example, mice, rats, pigs, non-human primates, and humans.

Extensive pre-clinical studies on the safety, toxicity, and efficacy of SCs in rodent and large animal paradigms of SCI have been performed. SCs may be safe and tolerated in treatment of sub-acute human SCI, chronic SCI, thoracic SCI, and cervical SCI, for complete and incomplete injuries.

SCs may facilitate anatomical repair and improvements in functional recovery after PNI and following transplantation into a range of experimental SCI paradigms. However, administered alone, they may be unable to provide full restoration of lost function to the injured CNS. Deficiencies to SCI repair conventionally incur a loss of up to 85% cells after transplantation into the injured spinal cord, a common issue across all types of cell transplantation. Cell death limits capacity for supporting supraspinal axon growth, e.g. corticospinal tract (CST) axons, to guide axons beyond the lesion site into the caudal cord.

As disclosed herein, some or all of these limitations may be overcome by providing a supportive matrix and/or modifying SCs to imbue them with scar avoidance properties. These modifications have resulted in enhanced repair and functional recovery with SCs after SCI.

### Example 8: Enhancing Schwann Cell Survival and Axon Growth Support after Spinal Cord Injury

In the injured PNS, SCs that have lost contact with axons may respond to neuroregulin-1 (NRG1) released from the injured nerves, which in turn may stimulate prodigious SC proliferation and migration towards repopulation of the injured segment. In contrast, SCs transplanted into the injured spinal cord have shown little evidence of proliferation as measured by the cell division marker Ki-67. Therefore, the number of transplanted SCs that persist within the injured spinal cord is typically dependent upon the extent of survival within the injury milieu after injection.

Conventionally, transplanted SCs may be lost in response to any one or more of these cell death mechanisms: (1) shear stress and the resulting rupture of cell membranes during the injection procedure in which the cells are compressed against each other, the injection device, and/or the tissue at the site of deposition, (2) apoptosis caused by the disruption of cell-to-cell and cell-to-ECM contacts established during culture conditions, and (3) apoptosis or necrosis resulting from the exposure of cells to a diversity of cytotoxic molecules within the injury milieu. Whereas the use of drug inhibitors of cell death, targeting calpains or caspases, may reduce, retard, prevent, or inhibit at least some transplanted cell death, the use of biomaterials or matrices may provide cytoprotective features by being able to encapsulate cells during injection, provide anchoring and attachment, and/or restrict exposure of cells to molecules from the hostile environment of the lesion during the acute transplantation period.

SCs are transplanted in matrigel (mostly collagen and laminin), have shown a survival rate of about 36%, compared to non-seeded SCs which have shown a survival rate of about 14%. The rise in SC survival rate was accompanied by 2- to 3-fold increases in vascularization of the lesion-implant, axonal in-growth, and improvements in locomotion. The improvements also showed a positive correlation with the number of surviving SCs, indicating that the protective effects of the matrix on SC survival are likely involved in the improved repair and recovery.

Though matrigel significantly enhances the survivability and efficacy of SCs after SCI, the matrix is not clinically translatable. Despite the advantages of using scaffolds for cell delivery over direct cell injection, the types of scaffolds currently available are limited in their clinical utility due to costly and complex cell seeding procedures, challenges in manufacturing and shelf stability, concerns about potential disease transmission, poor ease of administration, and uncontrolled degradation. Currently available products do not possess physical properties that allow easy cell seeding and void filling. Consequently, it remains challenging to deliver therapeutic cells in a manner that is simple and effective, while overcoming their vulnerability to the cytotoxic environment of the lesion site.

The self-assembling peptide hydrogel disclosed herein has been shown to overcome these challenges. The self-assembling peptide scaffolding platform may be capable of preventing mechanical injury to the cells through shear-thinning properties. The matrix may be capable of providing anchorage for the SCs within the platform. The matrix may be capable of shielding the cells from cytotoxic molecules and inflammatory cytokines following transplantation to the injured spinal cord, thereby antagonizing effectors of anoikis, necrosis, and apoptosis during the acute transplantation period. The cell scaffolding matrix may be capable of allowing simple and rapid cell encapsulation at the point-of-care, as well as conformal filling of wounds (see FIG. 19), representing a significant improvement over existing biomaterials.

### Example 9: Providing Scar-Avoidance Activity to Schwann Cells and In-Growing Axons after Spinal Cord Injury

Intraspinal injection of SCs within the injured spinal cord may result in the long-term restriction of the transplanted cells and cell-derived materials to the site of deposition. This behavior of transplanted SCs after SCI appears analogous to the cellular interactions that occur normally at the dorsal root entry zone (DREZ), where astrocytes and the ECM produced may inhibit the entry of SCs into the spinal cord from the peripheral nerve roots. The restriction of SCs to the lesion typically prevents the cells and cell-derived material from reaching injured axons that have retracted from the lesion boundary, particularly supraspinal axons such as the CST, to guide them through the injury, as well as typically limits the ability of SCs to then chaperone those axons from the lesion into the contiguous caudal cord where they can make appropriate connections.

In addition, by not being able to leave the lesion, SCs cannot reach areas of distal demyelination to provide remyelination repair and restore axon conduction. The disruption of SC-astrocyte/ECM interactions through the surface modification of either cell type with polysialic acid (PSA) to block cell to cell interactions may remove the restriction of the glial scar on SC migration, enhance their reparative behavior, and/or improve functional recovery after SCI and SC transplantation. Similarly, the masking or removal of inhibitory chondroitin sulfate proteoglycans (CSPGs), such as using the enzyme ChaseABC, may remove the restriction of the glial scar on SC migration, enhance their reparative behavior, and/or improve functional recovery after SCI and SC transplantation. Though effective, these approaches may perturb the structural and barrier functions of CSPGs that are important for limiting the spread of inflammation and tissue injury or limit aberrant plasticity and synaptic activity.

The receptors involved in CSPG-mediated inhibitory actions on growth and migration include receptor protein tyrosine phosphatase σ (RPTPσ) and leukocyte common antigen-related (LAR). In particular, the expression of RPTPσ was found on injured dystrophic axons. Targeting of RPTPσ with a small peptide mimetic of the RPTPσ intracellular sigma peptide (ISP) wedge region may relieve CSPG inhibition, reduce motor neuron death, enhance axon regeneration across the glial scar, restore the neuromuscular junction, and/or improve recovery after SCI when delivered subcutaneously, for example, daily. SCs may also be employed to express RPTPσ, which may mediate the inhibitory action of CSPGs on their migration and integration within the CNS.

The scaffold platform of self-assembly peptides disclosed herein may be selected and amenable to functionalization with encapsulated or tethered peptides. The peptide matrix may be functionalized by immobilization, for example, achieved through inclusion of functional moieties of angiogenic bioactive motifs such as vascular endothelial growth factor (VEGF), as well as cellular adhesion domains (e.g., RGD, IKVAV, YIGSR), to its amino acid self-assembling peptide sequence (Tables 3-5). The peptide matrix may be functionalized by non-immobilization, for example, achieved through encapsulation and controlled delivery of small and large molecule therapeutics (e.g., NGF, BDNF etc.) from its peptide matrix. These methods may enable the functional materials disclosed herein to act as drug delivery platforms, extending release over predetermined periods of time to potentially control cell and tissue fate. The release may be extended, for example, days, weeks, months, or years.

To modify the self-assembling peptide matrix and allow it to imbue injured axons and transplanted SCs with scar avoidance activity after SCI, the matrix may be functionalized with encapsulating anti-scar agents (for example, ISP), allowing the sustained release of the peptide to the lesion-transplant and adjacent host tissue for a therapeutic period. In some embodiments, sustained release may be achieved for about 1 month. Local delivery during the period of time required for SCs to migrate into the adjacent host tissue and for injured axons to transverse the lesion-transplant and into the contiguous caudal region offers advantages over repetitive systemic administration, which would require high and expensive peptide concentrations, potentially longer hospital stays, and produce aberrant or off-target effects in other organ systems or the CNS, where CSPG-controlled plasticity, joint/bone formation and other processes are fine-tuned physiologically. The disclosed scar avoidance functionalized cell scaffolding matrix technology may allow the regeneration of axons through the lesion site and permit encapsulated SCs to migrate outward to guide axons across the lesion as well as reach areas of distal demyelination to provide remyelination repair, representing a significant improvement over existing biomaterials.

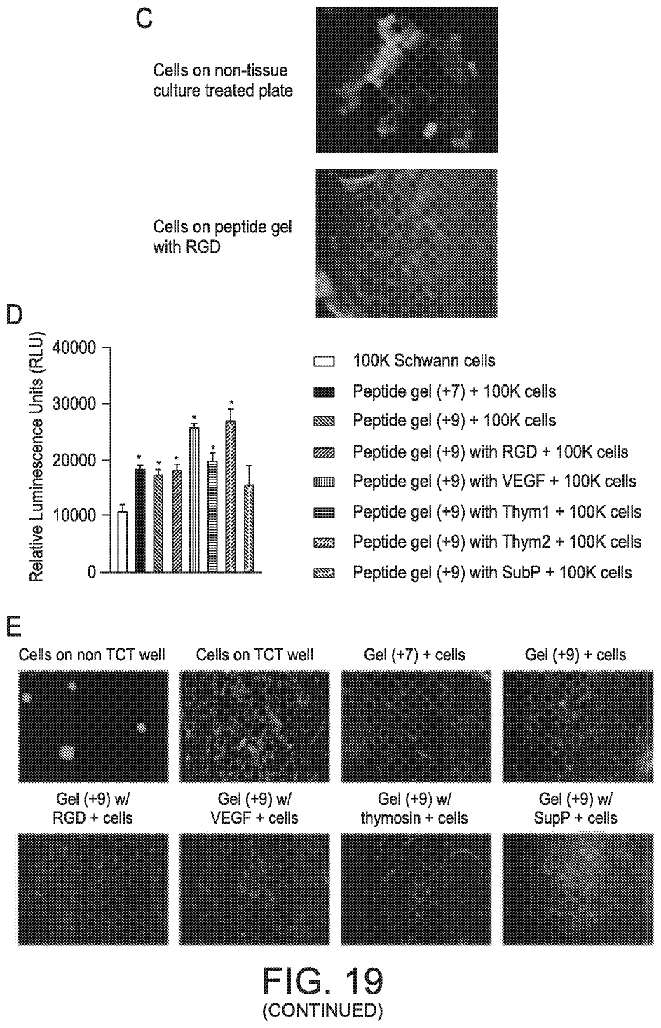

Antimicrobial efficacy of the peptide hydrogel is shown in the images and graphs of FIG. 20. Briefly, antimicrobial efficacy of the self-assembling peptide hydrogel was shown to increase with increasing electrostatic charge. The images in FIG. 20 show methicillin-resistant Staphylococcus aureus (MRSA, ATCC 33591) (n=6) (images A-C) or Pseudomonas aeruginosa (PA01) (N=3) (images E-G) cultured on BHI agar plates with the peptide hydrogels of differing charge. A+9 charge hydrogel (images A, E), a +7 charge hydrogel (images B, F), and a +5 charge hydrogel (images C, G). the peptide hydrogels with +9 and +7 charge had full clearance of 6-log MRSA (shown in image D) or PA01 (shown in image H) after 24 hours. The +5 hydrogel showed less anti-microbial efficacy than the +9 and +7 charge hydrogels.

The phraseology and terminology used herein is for the purpose of description and should not be regarded as limiting. As used herein, the term “plurality” refers to two or more items or components. The terms “comprising,” “including,” “carrying,” “having,” “containing,” and “involving,” whether in the written description or the claims and the like, are open-ended terms, i.e., to mean “including but not limited to.” Thus, the use of such terms is meant to encompass the items listed thereafter, and equivalents thereof, as well as additional items. Only the transitional phrases “consisting of” and “consisting essentially of,” are closed or semi-closed transitional phrases, respectively, with respect to the claims. Use of ordinal terms such as “first,” “second,” “third,” and the like in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements.

Having thus described several aspects of at least one embodiment, it is to be appreciated various alterations, modifications, and improvements will readily occur to those skilled in the art. Any feature described in any embodiment may be included in or substituted for any feature of any other embodiment. Such alterations, modifications, and improvements are intended to be part of this disclosure and are intended to be within the scope of the invention. Accordingly, the foregoing description and drawings are by way of example only.

Those skilled in the art should appreciate that the parameters and configurations described herein are exemplary and that actual parameters and/or configurations will depend on the specific application in which the disclosed methods and materials are used. Those skilled in the art should also recognize or be able to ascertain, using no more than routine experimentation, equivalents to the specific embodiments disclosed.

Supplementary Table 1: Selected Peptide Sequences

| SEQ ID | | | |
|---|---|---|---|
| and | | | Modification |
| length | Description | Sequence | and location |
| SEQ ID | G4D_0R; | VKVKVKVKVPPTKVKVKVKV | D-amino |
| NO: 1 | Synthetic | | acid |
| (20 aa) | Peptide | | (10)...(10) |
| SEQ ID | G4D_6R_v1; | VKVRVRVRVPPTRVRVRVKV | D-amino |
| NO: 2 | Synthetic | | acid |
| (20 aa) | Peptide | | (10)...(10) |
| SEQ ID | G4D_6R_v1 | VKVRVRVRVPPTRVRVRVKVGRGDSP | D-amino |
| NO: 3 | RGD; | | acid |
| (26 aa) | Synthetic | | (10)...(10) |
| | Peptide | | |
| SEQ ID | G4D_6R_v4; | VRVRVKVRVPPTRVEVRVRV | D-amino |
| NO: 4 | Synthetic | | acid |
| (20 aa) | Peptide | | (10)...(10) |
| SEQ ID | G4D_6R_v2; | VRVRVRVKVPPTKVRVRVRV | D-amino |
| NO: 5 | Synthetic | | acid |
| (20 aa) | Peptide | | (10)...(10) |
| SEQ ID | G4D_6R_v2_ | RGDSVRVRVRVKVPPTKVRVRVRV | D-amino |
| NO: 6 | RGD; | | acid |
| (24 aa) | Synthetic | | (14)...(14) |
| | Peptide | | |
| SEQ ID | G4D_6R_v1_ | VKVRVRVRVPTRVRVRVKVGKLTWQELYQLKYKGI | D-amino |
| NO: 7 | VG; | | acid |
| (36 aa) | Synthetic | | (10)...(10) |
| | Peptide | | |
| SEQ ID | G4D_6R_v1_ | VKVRVRVRVPPTRVRVRVKVRPKPQQFFGLM | D-amino |
| NO: 8 | SP; | | acid |
| (31 aa) | Synthetic | | (10)...(10) |
| | Peptide | | |
| SEQ ID | G4D_6R_v1_ | VKVRVRVRVRVRVRVKV | |
| NO: 9 | Ntrn; | | |
| (17 aa) | Synthetic | | |
| | Peptide | | |
| SEQ ID | G4D_2R_v1; | VKVKVRVKVPPTKVKVRVKV | D-amino |
| NO: 10 | Synthetic | | acid |
| (20 aa) | Peptide | | (10)...(10) |
| SEQ ID | G4D_6R_V3; | VKVRVRVRYNGTRVRVRVKV | |
| NO: 11 | Synthetic | | |
| (20 aa) | Peptide | | |
| SEQ ID | G4D_V1_ | LKKTETQGVKVRVRVRVPPTRVRVRVKV | D-amino |
| NO: 12 | LKKTETQ; | | acid |
| (28 aa) | Synthetic | | (18)...(18) |
| | Peptide | | |
| SEQ ID | G4D_V1_ | SDKPGVLVRVRVRVPPTRVRVRVKV | D-amino |
| NO: 13 | SDKP; | | acid |
| (25 aa) | Synthetic | | (15)...(15) |
| | Peptide | | |
| SEQ ID | G4D_6R_v2_ | RGDSVRVRVRVKVPPTKVRVRVRV | D-amino |
| NO: 14 | RGD; | | acid |
| (24 aa) | Synthetic | | (14)...(14) |
| | Peptide | | |
| SEQ ID | G4D_0R_E_ | VKVKVKVKVPPTKVEVKVKV | D-amino |
| NO: 15 | MAX8; | | acid |
| (20 aa) | Synthetic | | (10)...(10) |
| | Peptide | | |
| SEQ ID | G4D_HLT2; | VLTKVKTKVPPTKVEVKVLV | D-amino |
| NO: 16 | Synthetic | | acid |
| (20 aa) | Peptide | | (10)...(10) |
| SEQ ID | G4D_HLT2_ | VLTKVKTKVPPTKVEVKVLVGGGGRGDV | D-amino |
| NO: 17 | RGD; | | acid |
| (28 aa) | Synthetic | | (10)...(10) |
| | Peptide | | |
| SEQ ID | G4D_6R_v1_ | RGDSVKVRVRVRVPPTRVRVRVKV | D-amino |
| NO: 18 | RGD; | | acid |
| (24 aa) | Synthetic | | (14)...(14) |
| | Peptide | | |
| SEQ ID | G4D_6R_v1_ | IKVAVGVKVRVRVRVPPTRVRVRVKV | D-amino |
| NO: 19 | IKVAV; | | acid |
| (26 aa) | Synthetic | | (16)...(16) |
| | Peptide | | |
| SEQ ID | G4D_6R_v1_ | YIGSRGVKVRVRVRVPPTRVRVRVKN | D-amino |
| NO: 20 | YISGR; | | acid |
| (26 aa) | Synthetic | | (16)...(16) |
| | Peptide | | |
| SEQ ID | Synthetic | VLTKVKTVPPTKVEVKVL | D-amino |
| NO: 21 | Peptide 21 | | acid |
| (18 aa) | | | (9)...(9) |
| SEQ ID | Synthetic | VEVSVSVEVPPTEVSVEVEVGGGGRGDV | D-amino |
| NO: 22 | Peptide 22 | | acid |
| (28 aa) | | | (10)...(10) |
| SEQ ID | Synthetic | VEVSVSVEVPPTEVSVEVEV | D-amino |
| NO: 23 | Peptide 23 | | acid |
| (20 aa) | | | (10)...(10) |

1. A method of treating a nerve injury, comprising:
administering to the target site a thermally stable preparation comprising a purified amphiphilic peptide in an aqueous biocompatible solution, the peptide comprising a folding group having a plurality of charged amino acid residues and hydrophobic amino acid residues arranged in a substantially alternating pattern and a turn sequence, the peptide being configured to self-assemble into a hydrogel; and
administering to the target site a buffer comprising an effective amount of an ionic salt and a biological buffering agent to form the hydrogel.

2. A method of treating a nerve injury, comprising:
administering to a target site of the nerve injury a biological material suspension in an amount effective to treat the nerve injury;
administering to the target site an anti-scarring agent in an amount effective to treat the nerve injury;
administering to the target site of the nerve injury a thermally stable preparation comprising a purified amphiphilic peptide in an aqueous biocompatible solution, the peptide comprising a folding group having a plurality of charged amino acid residues and hydrophobic amino acid residues arranged in a substantially alternating pattern and a turn sequence, the peptide being configured to self-assemble into a hydrogel; and
administering to the target site a buffer comprising an effective amount of an ionic salt and a biological buffering agent to form the hydrogel.

3. The method of claim 1 or claim 2, wherein the target site of the nerve injury is associated with a central nervous system (CNS) tissue or a peripheral nervous system (PNS) tissue.

4. The method of claim 3, wherein the nerve injury is spinal cord injury (SCI).

5. The method of claim 3, wherein the nerve injury is peripheral nerve injury (PNI).

6. The method of claim 1, further comprising administering to the target site a biological material suspension, in an amount effective to treat the nerve injury.

7. The method of claim 4 or claim 5, wherein the preparation is administered in response to an SCI symptom or trigger, or a PNI symptom or trigger.

8. The method of claim 2 or claim 6, wherein the biological material comprises at least one of cells, cell-derived material, tissue, and tissue-derived material.

9. The method of claim 8, wherein the cells, the cell-derived material, the tissue, and/or the tissue-derived material is autologous, allogeneic, or xenogeneic.

10. The method of claim 9, further comprising obtaining the cells, the cell-derived material, the tissue, and/or the tissue-derived material from a donor.

11. The method of claim 9, further comprising obtaining the cells, the cell-derived material, the tissue, and/or the tissue-derived material from the subject.

12. The method of claim 8, wherein the cells comprise at least one of stem cells and glial cells.

13. The method of claim 12, wherein the stem cells comprise at least one of bone marrow derived stromal cells and adipose derived stromal cells.

14. The method of claim 12, wherein the stem cells comprise at least one of embryonic stem cells and adult stem cells.

15. The method of claim 12, wherein the glial cells comprise at least one of oligodendrocytes, astrocytes, ependymal cells, microglia, Schwann cells, and satellite cells.

16. The method of claim 8, wherein the cell-derived material or the tissue-derived material comprises at least one of exosomes and lysosomes.

17. The method of claim 1, further comprising administering an effective amount of an anti-scarring agent.

18. The method of claim 2 or claim 17, wherein the anti-scarring agent comprises at least one of receptor protein tyrosine phosphatase σ (RPTPσ) inhibitory peptide (ISP), chondroitinase ABC (ChaseABC), and polysialyl transferase (PST).

19. The method of claim 1 or claim 2, further comprising administering an effective amount of an axon regeneration agent.

20. The method of claim 19, wherein the axon regeneration agent comprises at least one of nerve growth factor (NGF), brain-derived neurotrophic factor (BDNF), glial cell line-derived neurotrophic factor (GDNF), vascular endothelial growth factor (VEGF), calcium, neurotrophin-3, interleukin-1 (IL-1), neuregulin, growth associated protein 43 (GAP-43), tubulin, actin, and heat-shock protein-27 (HSP-27).

21. The method of claim 1 or claim 2, wherein the subject is in need of treatment for the nerve injury, or the subject has been diagnosed with the nerve injury.

22. The method of claim 1 or claim 2, wherein the nerve injury is mild, moderate, or severe.

23. The method of claim 1 or claim 2, wherein the nerve injury is acute or chronic.

24. The method of claim 1 or claim 2, wherein the amount and/or frequency of administration is sufficient to promote treatment of the nerve injury.

25. The method of claim 24, wherein the amount and/or frequency of administration is sufficient to promote axon growth, axon migration, axon proliferation, axon alignment, axon regeneration, axon re-innervation, and/or axon attachment at the target site.

26. The method of claim 24, wherein the amount and/or frequency of administration is sufficient to form a scaffold and promote at least one of cell attachment and cell migration from the target site to a site of migration.

27. The method of claim 24, wherein the amount and/or frequency of administration is sufficient to promote bridging or void filling the target site of the nerve injury, and/or reducing or preventing scar formation at the target site of the nerve injury.

28. The method of any of claims 1, 2, 6, and 17, comprising administering the biological material suspension, the anti-scarring agent, the preparation, and/or the buffer topically, parenterally, or enterally.

29. The method of claim 28, wherein parenteral administration comprises administration to the target site by injection or by infusion.

30. The method of claim 29, wherein the biological material suspension, the anti-scarring agent, the preparation, and/or the buffer is injected via a minimally invasive procedure selected from intravenous, intrasecal, intramuscular, subcutaneous, intradermal, intramedullary, intravascular, intraventricular, intrabiliary, intrathecal, or epidural administration.

31. The method of claim 28, comprising administering the biological material suspension, the anti-scarring agent, the preparation, and/or the buffer topically to the target site by spray, dropper, film, squeeze tube, or syringe.

32. The method of claim 28, comprising combining two or more of the biological material suspension, the anti-scarring agent, the preparation, and the buffer prior to administration.

33. The method of claim 32, comprising combining the two or more of the biological material suspension, the anti-scarring agent, the preparation, and the buffer, less than about 1 minute, less than about 2 minutes, less than about 5 minutes, or less than about 10 minutes prior to administration.

34. The method of claim 32, comprising combining the two or more of the biological material suspension, the anti-scarring agent, the preparation, and the buffer, at a point of use.

35. The method of claim 28, wherein the biological material suspension, the anti-scarring agent, the preparation, and the buffer are administered separately.

36. The method of claim 28, comprising combining an axon regeneration agent with one or more of the biological material suspension, the preparation, and the buffer prior to administration.

37. The method of claim 1 or claim 2, wherein the target site is a tissue selected from mesenchymal tissue, connective tissue, muscle tissue, nervous tissue, embryonic tissue, dermal tissue, bone tissue, dental tissue, corneal tissue, cutaneous tissue, integumental tissue, soft tissue, hard tissue, and combinations thereof.

38. The method of claim 37, wherein the target site is associated with a desired local effect.

39. The method of claim 1 or claim 2, wherein the peptide comprises an effective amount of counterions.

40. The method of claim 39, wherein the peptide comprises an effective amount of acetate, citrate, and/or chloride counterions.

41. The method of claim 1 or claim 2, wherein the peptide is substantially free of chloride counterions.

42. The method of claim 1 or claim 2, wherein the buffer comprises between about 10 mM and 150 mM sodium chloride and between about 10 mM and 100 mM Bis-tris propane (BTP).

43. The method of any of claims 1, 2, 6, and 17, comprising administering the biological material suspension, the anti-scarring agent, the preparation, and/or the buffer in combination with a surgical procedure.

44. The method of any of claims 1, 2, 6, and 17, comprising administering a first dosage of the biological material suspension, the anti-scarring agent, the preparation, and/or the buffer.

45. The method of claim 44, comprising administering at least one booster dosage of the biological material suspension, the anti-scarring agent, the preparation, and/or the buffer.

46. The method of claim 1 or claim 2, wherein:
the hydrophobic amino acid residues are independently selected from glycine, alanine, valine, leucine, isoleucine, proline, phenylalanine, methionine, threonine, tryptophan, and combinations thereof; and
the charged amino acid residues are independently selected from arginine, lysine, histidine, and combinations thereof.

47. The method of claim 46, wherein the folding group has a sequence comprising Y[XY]_{N}[T][YX]_{M}Y, where X is 1-3 charged amino acids, Y is 1-3 hydrophobic amino acids, T is 2-8 turn sequence amino acids, and N and M are each independently between 2 and 10.

48. The method of claim 46, wherein the turn sequence amino acids are independently selected from a D-proline, an L-proline, aspartic acid, threonine, asparagine, and combinations thereof.

49. The method of claim 1 or claim 2, wherein the peptide is configured to self-assemble into a substantially biocompatible hydrogel.

50. The method of claim 49, wherein the peptide is configured to self-assemble into a hydrogel having at least one property selected from:
a cell friendly hydrogel;
a substantially biodegradable, non-inflammatory, and/or non-toxic hydrogel;
a hydrogel having substantially low hemolytic activity; and
a hydrogel having substantially low immunogenic activity.

51. The method of any of claims 1, 2, 6, and 17, further comprising administering at least one combination treatment selected from: an antibacterial composition, an antifungal composition, an antiviral composition, an anti-tumor composition, an anti-inflammatory composition, a cell culture media, a cell culture serum, an anti-odor composition, a hemostatic composition, and an analgesic or pain-relief composition.

52. The method of claim 51, wherein the combination treatment is administered prior to the preparation.

53. The method of claim 51, wherein the combination treatment is administered after the preparation.

54. The method of claim 51, wherein the combination treatment is administered concurrently with the preparation.

55. The method of claim 1 or claim 2, wherein the peptide is at least 80% purified, for example, at least 85%, at least 90%, at least 92%, at least 95%, at least 98%, at least 99%, or at least 99.9%.

56. The method of claim 55, wherein the peptide has less than 10% residual organic solvent by weight, for example, less than 8%, less than 5%, less than 2%, less than 1%, or less than 0.1%.

57. The method of claim 56, wherein the organic solvent comprises at least one of trifluoroacetic acid (TFA), acetonitrile, isopropanol, N,N-Dimethylformamide, triethylamine, Ethyl Ether, and acetic acid.

58. The method of claim 57, wherein the preparation has a residual Trifluoroacetic acid (TFA) concentration of less than about 1% w/v, a residual acetonitrile concentration of less than about 410 ppm, a residual N,N-Dimethylformamide concentration of less than about 880 ppm, a residual triethylamine concentration of less than about 5000 ppm, a residual Ethyl Ether concentration of less than about 1000 ppm, a residual isopropanol concentration of less than about 100 ppm, and/or a residual acetic acid concentration of less than 0.1% w/v.

59. The method of claim 1 or claim 2, wherein the peptide includes a functional group.

60. The method of claim 59, wherein the functional group has between 3 and 30 amino acid residues.

61. The method of claim 59, wherein the functional group is engineered to express a bioactive property.

62. The method of claim 59, wherein the functional group is engineered to control or alter charge or pH of the peptide or preparation.

63. The method of claim 59, wherein the functional group is engineered for a target indication, e.g., selected from cell culture, cell delivery, wound healing, treatment of biofilm, and combinations thereof.

64. The method of claim 59, wherein the functional group has a sequence selected from RGD, IKVAV, YIGSR, LKKTETQ, SNKPGVL, PKPQQFFGLM, GKLTWQELYQLKYKGI, and GGG.

65. The method of claim 1 or claim 2, wherein the peptide is configured to self-assemble into a substantially ionically-crosslinked hydrogel.

66. The method of claim 1 or claim 2, wherein the peptide is configured to self-assemble into a shear-thinning hydrogel.

67. The method of claim 1 or claim 2, wherein the peptide is configured to self-assemble into a substantially transparent hydrogel.

68. The method of claim 1 or claim 2, wherein the buffer comprises from about 5 mM to about 200 mM ionic salts.

69. The method of claim 68, wherein the ionic salt dissociates into at least one of sodium, potassium, calcium, magnesium, iron, ammonium, pyridium, quaternary ammonium, chloride, and sulfate ions.

70. The method of claim 69, wherein the ionic salts comprise sodium chloride, ammonium chloride, magnesium chloride, potassium chloride, calcium chloride, ammonium sulfate, magnesium sulfate, sodium sulfate, potassium sulfate, calcium sulfate, sodium bicarbonate, and combinations thereof.

71. The method of claim 70, wherein the buffer comprises from about 10 mM to about 150 mM sodium chloride.

72. The method of claim 1 or claim 2, wherein the peptide has a bacterial endotoxin level of less than about 10 EU/mg.

73. The method of claim 1 or claim 2, wherein the preparation comprises between 0.1% w/v and 8.0% w/v of the peptide.

74. The method of claim 73, wherein the preparation comprises between 0.5% w/v and 6.0% w/v of the peptide, for example, between 0.5% w/v and 3.0% w/v of the peptide, between 0.5% w/v and 1.5% w/v of the peptide between 0.5% w/v and 1.0% w/v of the peptide, or between 0.7% w/v and 0.8% w/v of the peptide.

75. The method of claim 74, wherein the hydrogel comprises between 0.25% w/v and 6.0% w/v of the peptide.

76. The method of claim 1 or claim 2, wherein the peptide is configured to self-assemble into a hydrogel having between 90% w/v and 99.9% w/v aqueous solution.

77. The method of claim 1 or claim 2, wherein the peptide has a net charge of from −7 to +11.

78. The method of claim 77, wherein the peptide has a net charge of from +2 to +9, for example, from +5 to +9.

79. The method of claim 1 or claim 2, wherein the peptide is lyophilized.

80. The method of claim 1 or claim 2, wherein the preparation is sterile.

81. The method of claim 80, wherein the preparation is substantially free of a preservative.

82. The method of claim 1 or claim 2, wherein the preparation is thermally stable between-20° C. and 150° C.

83. The method of claim 82, wherein the preparation is sterilized by autoclave sterilization.

84. The method of any of claims 1, 2, 6, and 17, comprising providing at least one of the biological material suspension, the anti-scarring agent, the preparation, and the buffer.

85. The method of any of claims 1, 2, 6, and 17, comprising providing at least one of the biological material suspension, the anti-scarring agent, the peptide, the biocompatible solution, and the buffer separately.

86. A method of preparing a nerve injury treatment composition, comprising:
combining a therapeutically effective amount of a biological material suspension with:
a preparation comprising a purified amphiphilic peptide comprising a folding group having a plurality of charged amino acid residues and hydrophobic amino acid residues arranged in a substantially alternating pattern and a turn sequence, the peptide being configured to self-assemble into a hydrogel, and
a buffer comprising an effective amount of an ionic salt and a biological buffering agent to form the hydrogel.

87. The method of claim 86, comprising:
combining the biological material suspension with the preparation to produce a biological material peptide preparation, and
combining the biological material peptide preparation with the buffer to form the hydrogel.

88. The method of claim 86, comprising:
combining the biological material suspension with the buffer to produce a biological material buffer suspension, and
combining the biological material buffer suspension with the preparation to form the hydrogel.

89. The method of claim 86, comprising:
combining the preparation with the buffer to form the hydrogel, and
combining the biological material suspension with the hydrogel to produce the nerve injury treatment composition.

90. The method of claim 86, further comprising combining the biological material suspension with an anti-scarring agent.

91. The method of claim 86 or claim 90, comprising combining at least two of the biological material suspension, the anti-scarring agent, the preparation, and the buffer in vitro.

92. The method of claim 86 or 90, comprising combining at least two of the biological material suspension, the anti-scarring agent, the preparation, and the buffer in vivo.

93. The method of claim 92, comprising combining the at least two of the biological material suspension, the anti-scarring agent, the preparation, and the buffer in situ.

94. The method of claim 91, comprising combining the preparation with the buffer to form the hydrogel in vitro, and combining the biological material suspension with the hydrogel in vivo.

95. The method of claim 86, wherein the biological material suspension comprises at least one of cells, cell-derived materials, tissue, and tissue-derived materials, the method further comprising culturing the biological material in the hydrogel for a predetermined period of time prior to administration to a subject.

96. The method of claim 95, wherein the hydrogel comprises a non-homogeneous suspension of the cells, e.g., comprising clusters or spheroids of the cells.

97. The method of claim 95, further comprising combining the biological material suspension with a cell culture media, cell maintenance agent, cell growth agent, cell culture serum, or combination thereof.

98. A method of facilitating treatment of a nerve injury in a subject, comprising:
providing a preparation comprising a purified amphiphilic peptide comprising a folding group having a plurality of charged amino acid residues and hydrophobic amino acid residues arranged in a substantially alternating pattern and a turn sequence, the peptide being configured to self-assemble into a hydrogel;
providing instructions to combine biological material with the preparation and a buffer comprising an effective amount of an ionic salt and a biological buffering agent to form the hydrogel;
providing instructions to agitate the hydrogel comprising the biological material to produce a biological material suspension hydrogel; and
providing instructions to administer an effective amount of the biological material suspension hydrogel to a target site of the nerve injury to provide treatment of the nerve injury to the subject.

99. The method of claim 98, further comprising providing the buffer.

100. The method of claim 98, further comprising providing the biological material.

101. The method of claim 100, wherein the biological material comprises at least one of cells, cell-derived materials, tissue, and tissue-derived materials.

102. The method of claim 98, wherein administering the effective amount of the biological material suspension hydrogel to the target site provides treatment of spinal cord injury (SCI).

103. The method of claim 98, wherein administering the effective amount of the biological material suspension hydrogel to the target site provides treatment of peripheral nerve injury (PNI).

104. The method of claim 98, further comprising providing at least one of a mixing device configured to agitate the hydrogel and a delivery device configured to administer the biological material suspension hydrogel.

105. A kit comprising:
a biological material suspension;
